SEQUENCE LISTING (35 sequences)

SEQ ID NO: 1 - DNA, 2134 bp, Oryza sativa
gaaactaccagaatgcccgtatgttgcaacacgattttaattaattaaagaaaatatcattaatctattattataatcttttaattcaattgatgtagatatttgttcctgacttgtggatttttttagtctcacactctcccttttttttctttctcatgggagtggtcgatatgtgcagcgtctaacaccacatgtcctttttcttcgaaaaaagagttctcccttataacttttataaggagtatagatagtttgcattccttgcttgctgctttttttatagatataatttgcatttccttgttgcactgatcattagctcattagttcatcccatgaggacatgatgacagtatgacactacccagaatgattatttttcacttaatatttggagatttatctcgacgtcactgggtggcatataagggatgcgacaaacactccatatcaatcgaggaatttcctctcgtcttgaattatgactgatcatgacttgtcattagagaagttaaagaaaaagatgggacttatcaggagagactaaaaaaagagtagccctacgagtagtttagtgtgttttgccatactaccatgtgatggtgatgtcaaaaacatggggctcgcgtaaccgcaaaactaaggctgtcccagacacactaatcaaaagcattcgaatttagaactcctataatttgttagtttgtttaatcactctttgtgtctcatgatgattcatgcttgcataattgcatacacttctcgactctaagcttggaccacacaacccgtacataggacgaaaccgaaagcatcacccaaaaaaaaaaaaactaatgtgcccctctttagcatgtcagaataatatggaaaaggcgtcgagcagaaacaaatatgtcaagagtttgacaaacctgacaactgccaatgcagccaatgagccaagcagcaacttgttgatggctggtaggctttcccattatattctggcaaaaactttgcttccatttttctattgacgacgatgagttcgccgattccggccgggccggctcagcttttctccttcggctagcgtttggacgcacacttttgagagaatcgaaacacggagagaagtgctccgatgcatgacacctccttttccacgtacttaacgccaaagggaaacaatgatatgcagtgaacacaaagggattgaccctgcccaggatcatcaatacacgttgaggcttcttcgtgtgtgaaagcatggctaactcgtctggtcaaaggcaaaattgctgctatcctaaaacaaatagtactaggataaataaatgaaagggctaatataacactcgcacacacgcgagacgacaaaatggagagctagcacacacgatagggacggctcgacggtaggggaggaggaataatgcaggcaggatgttactatggccacgtttagttggttagtgtgacaaaaaatttttaaaagtatacagacatacataatatatatttaaagtattaaaggtagactaataacaaaataaattacagatttcacatataaactgtgaggtgaatttattaagtttaattaatccttcattagcaaatgtttactgtagcatcacattgtcaaatcatggcgtaattagactcaaaagattcgtttcgcaatttacatgcaaactgtgcaattggtttttttccatatttaatgcttcatatatgtattcaaatatttgatgtgacggaattttttaaagtttaaattcaaatatttgatgtgacggaattttttaaagtttgaagggaaataaacacggcctatggcgcaaaattgtactacaaactccctttcgcgagacgagagaatgcaacgccgtgccgctctggcggctggggcccaaccgcgaaaggcaatccatccctctctctccccgtcgcgtaaggccgctataaatcaacccgtcgcgggttgcattgcatcggtcggtctcggtcgctgccgtccctgtgcttggagtagacacgagccggtgacgcgcatatcgtcttctgctcgagatcgatcgatcgatcgcttggttggttgctgcgactgcgagggaggccgcgggtggtgaggaggattgtgcaa

SEQ ID NO: 2 - DNA, 4061 bp, Oryza sativa
gatttggatgaacttgggttgcatttgggctttctatatgggcctgaatttgaatttccttgggatgcaaaattctctcgaggaaaaaagtacaccgaaggtccctcaacttggcatcgagttacaaaatcgtcccccaaccacaaaaccggatacaacgcatcccccaacttataaaaccagtgcatactgatcccggttttagctgacatggcggctgactcagcgtggaacccacgtgggccccatatgtcaggatgccacgtcggccagcctctcccttcctttcctgtcccctcctttcgtctcgacttgttaggatgcaacgtcatcagcagggcggccggcggtggggagggggttgccagggtgagaggtccgacggccggcgggcgatgcggcggcggcaacgtggcggctgagacacgagagaaggggaaggcagcggaccagcgttgtggcggagaggcggcagcaggcgtgcgagtgtcgtggcggagagggcggaggcggtagctgcggcggagaggcgatggcggcggatgcgagcgtcgtggcggagaggaggcggcggcggcggctgccacgcgggagggcaggccgaaggcgacggcgtgggcgagcgtggaggcacgcgacggcgaggctgccgctccgcccgtcgcctcttctctctcaggtttctcctcgtctgggtgggcaggtcggccggcgggttgggaaggaagggaaggggttggggtgatggcttgctggagctgcttcggctgcagccggtcgatcgcagcaggaggcgaggtccggctgccggagccgttccagctgccggcgcccttgcccagctagccgcatggtctagggacttgttcgctttcatcgttcttcgaattctctccccgtttcttcgtgtactgtttaattggttcttaatctgctcataaattaatcgtcgtcatctatggcctggattgccggatagtatctttgtgcttgtaaattttcagcggctatgcgagagctcgtgccaaatttgatatgcgagagttttgttgtttgtttcgccggcgaccgagcaggacgggtccgggcaccggagcgacaggcaccgcgcgccgtcgcccaccgccacgcggaaacgaccggtgatgcggcagccgcaccggcctgccggaggaccgggcaaatggtgggcgaagcgggcaacgacgcgggccggagtggccggccggcggtgggcaggcacgctgcgggagaggaaggggctgcggcggcaggggcgcagaaggtcaggccgcaccggcaggcggctgtcgacatcgaccatttagagggcagagagaggacaagaacggggagaggagagagcggaggcggatggcgccgacggggagcgggatggggatagcaaggccgtcatcgccgtcgtcgtccatggcgtggggaggccgcctccgctccgcctgtcgcctgcctgccgccgccgacccgcctgcccaccggccgccgaacctcctcctctcccgcctcgccccgtcggcttcctccccaacccgtcggccggcctgcccacccagacgaggtgaaagctgagagagaagaggggaaagagagagatgacgtggcatcctgacatgtgggttccacgctgacatgtgggttccacgctgagtcagctgctacgtcggaaaaaactgaggtcaaaaccgccgagggacctagtttgccctggttttgtaagttggaggatgcgttgtatccggttttgcggttggaggacgattttgtaactcgatgacaagttgagggaccttcggtgtactttttccttctctcgatgccactggattgggccggtttggaccaatttgcgtgcattttgagctttccatgtgggccgtagtttgcattccttttttttttgagaaactaccagaatgcccgtatgttgcaacacgattttaattaattaaagaaaatatcattaatctattattataatcttttaattcaattgatgtagatatttgttcctgacttgtggatttttttagtctcacactctcccttttttttctttctcatgggagtggtcgatatgtgcagcgtctaacaccacatgtcctttttcttcgaaaaaagagttctcccttataacttttataaggagtatagatagtttgcattccttgcttgctgctttttttatagatataatttgcatttccttgttgcactgatcattagctcattagttcatcccatgaggacatgatgacagtatgacactacccagaatgattatttttcacttaatatttggagatttatctcgacgtcactgggtggcatataagggatgcgacaaacactccatatcaatcgaggaatttcctctcgtcttgaattatgactgatcatgacttgtcattagagaagttaaagaaaaagatgggacttatcaggagagactaaaaaaagagtagccctacgagtagtttagtgtgttttgccatactaccatgtgatggtgatgtcaaaaacatggggctcgcgtaaccgcaaaactaaggctgtcccagacacactaatcaaaagcattcgaatttagaactcctataatttgttagtttgtttaatcactctttgtgtctcatgatgattcatgcttgcataattgcatacacttctcgactctaagcttggaccacacaacccgtacataggacgaaaccgaaagcatcacccaaaaaaaaaaaaactaatgtgcccctctttagcatgtcagaataatatggaaaaggcgtcgagcagaaacaaatatgtcaagagtttgacaaacctgacaactgccaatgcagccaatgagccaagcagcaacttgttgatggctggtaggctttcccattatattctggcaaaaactttgcttccatttttctattgacgacgatgagttcgccgattccggccgggccggctcagcttttctccttcggctagcgtttggacgcacacttttgagagaatcgaaacacggagagaagtgctccgatgcatgacacctccttttccacgtacttaacgccaaagggaaacaatgatatgcagtgaacacaaagggattgaccctgcccaggatcatcaatacacgttgaggcttcttcgtgtgtgaaagcatggctaactcgtctggtcaaaggcaaaattgctgctatcctaaaacaaatagtactaggataaataaatgaaagggctaatataacactcgcacacacgcgagacgacaaaatggagagctagcacacacgatagggacggctcgacggtaggggaggaggaataatgcaggcaggatgttactatggccacgtttagttggttagtgtgacaaaaaatttttaaaagtatacagacatacataatatatatttaaagtattaaaggtagactaataacaaaataaattacagatttcacatataaactgtgaggtgaatttattaagtttaattaatccttcattagcaaatgtttactgtagcatcacattgtcaaatcatggcgtaattagactcaaaagattcgtttcgcaatttacatgcaaactgtgcaattggtttttttccatatttaatgcttcatatatgtattcaaatatttgatgtgacggaattttttaaagtttaaattcaaatatttgatgtgacggaattttttaaagtttgaagggaaataaacacggcctatggcgcaaaattgtactacaaactccctttcgcgagacgagagaatgcaacgccgtgccgctctggcggctggggcccaaccgcgaaaggcaatccatccctctctctccccgtcgcgtaaggccgctataaatcaacccgtcgcgggttgcattgcatcggtcggtctcggtcgctgccgtccctgtgcttggagtagacacgagccggtgacgcgcatatcgtcttctgctcgagatcgatcgatcgatcgcttggttggttgctgcgactgcgagggaggccgcgggtggtgaggaggattgtgcaaaaaaa

SEQ ID NO: 3 - DNA, 10000 bp, Oryza sativa
gatttggatgaacttgggttgcatttgggctttctatatgggcctgaatttgaatttccttgggatgcaaaattctctcgaggaaaaaagtacaccgaaggtccctcaacttggcatcgagttacaaaatcgtcccccaaccacaaaaccggatacaacgcatcccccaacttataaaaccagtgcatactgatcccggttttagctgacatggcggctgactcagcgtggaacccacgtgggccccatatgtcaggatgccacgtcggccagcctctcccttcctttcctgtcccctcctttcgtctcgacttgttaggatgcaacgtcatcagcagggcggccggcggtggggagggggttgccagggtgagaggtccgacggccggcgggcgatgcggcggcggcaacgtggcggctgagacacgagagaaggggaaggcagcggaccagcgttgtggcggagaggcggcagcaggcgtgcgagtgtcgtggcggagagggcggaggcggtagctgcggcggagaggcgatggcggcggatgcgagcgtcgtggcggagaggaggcggcggcggcggctgccacgcgggagggcaggccgaaggcgacggcgtgggcgagcgtggaggcacgcgacggcgaggctgccgctccgcccgtcgcctcttctctctcaggtttctcctcgtctgggtgggcaggtcggccggcgggttgggaaggaagggaaggggttggggtgatggcttgctggagctgcttcggctgcagccggtcgatcgcagcaggaggcgaggtccggctgccggagccgttccagctgccggcgcccttgcccagctagccgcatggtctagggacttgttcgctttcatcgttcttcgaattctctccccgtttcttcgtgtactgtttaattggttcttaatctgctcataaattaatcgtcgtcatctatggcctggattgccggatagtatctttgtgcttgtaaattttcagcggctatgcgagagctcgtgccaaatttgatatgcgagagttttgttgtttgtttcgccggcgaccgagcaggacgggtccgggcaccggagcgacaggcaccgcgcgccgtcgcccaccgccacgcggaaacgaccggtgatgcggcagccgcaccggcctgccggaggaccgggcaaatggtgggcgaagcgggcaacgacgcgggccggagtggccggccggcggtgggcaggcacgctgcgggagaggaaggggctgcggcggcaggggcgcagaaggtcaggccgcaccggcaggcggctgtcgacatcgaccatttagagggcagagagaggacaagaacggggagaggagagagcggaggcggatggcgccgacggggagcgggatggggatagcaaggccgtcatcgccgtcgtcgtccatggcgtggggaggccgcctccgctccgcctgtcgcctgcctgccgccgccgacccgcctgcccaccggccgccgaacctcctcctctcccgcctcgccccgtcggcttcctccccaacccgtcggccggcctgcccacccagacgaggtgaaagctgagagagaagaggggaaagagagagatgacgtggcatcctgacatgtgggttccacgctgacatgtgggttccacgctgagtcagctgctacgtcggaaaaaactgaggtcaaaaccgccgagggacctagtttgccctggttttgtaagttggaggatgcgttgtatccggttttgcggttggaggacgattttgtaactcgatgacaagttgagggaccttcggtgtactttttccttctctcgatgccactggattgggccggtttggaccaatttgcgtgcattttgagctttccatgtgggccgtagtttgcattccttttttttttgagaaactaccagaatgcccgtatgttgcaacacgattttaattaattaaagaaaatatcattaatctattattataatcttttaattcaattgatgtagatatttgttcctgacttgtggatttttttagtctcacactctcccttttttttctttctcatgggagtggtcgatatgtgcagcgtctaacaccacatgtcctttttcttcgaaaaaagagttctcccttataacttttataaggagtatagatagtttgcattccttgcttgctgctttttttatagatataatttgcatttccttgttgcactgatcattagctcattagttcatcccatgaggacatgatgacagtatgacactacccagaatgattatttttcacttaatatttggagatttatctcgacgtcactgggtggcatataagggatgcgacaaacactccatatcaatcgaggaatttcctctcgtcttgaattatgactgatcatgacttgtcattagagaagttaaagaaaaagatgggacttatcaggagagactaaaaaaagagtagccctacgagtagtttagtgtgttttgccatactaccatgtgatggtgatgtcaaaaacatggggctcgcgtaaccgcaaaactaaggctgtcccagacacactaatcaaaagcattcgaatttagaactcctataatttgttagtttgtttaatcactctttgtgtctcatgatgattcatgcttgcataattgcatacacttctcgactctaagcttggaccacacaacccgtacataggacgaaaccgaaagcatcacccaaaaaaaaaaaaactaatgtgcccctctttagcatgtcagaataatatggaaaaggcgtcgagcagaaacaaatatgtcaagagtttgacaaacctgacaactgccaatgcagccaatgagccaagcagcaacttgttgatggctggtaggctttcccattatattctggcaaaaactttgcttccatttttctattgacgacgatgagttcgccgattccggccgggccggctcagcttttctccttcggctagcgtttggacgcacacttttgagagaatcgaaacacggagagaagtgctccgatgcatgacacctccttttccacgtacttaacgccaaagggaaacaatgatatgcagtgaacacaaagggattgaccctgcccaggatcatcaatacacgttgaggcttcttcgtgtgtgaaagcatggctaactcgtctggtcaaaggcaaaattgctgctatcctaaaacaaatagtactaggataaataaatgaaagggctaatataacactcgcacacacgcgagacgacaaaatggagagctagcacacacgatagggacggctcgacggtaggggaggaggaataatgcaggcaggatgttactatggccacgtttagttggttagtgtgacaaaaaatttttaaaagtatacagacatacataatatatatttaaagtattaaaggtagactaataacaaaataaattacagatttcacatataaactgtgaggtgaatttattaagtttaattaatccttcattagcaaatgtttactgtagcatcacattgtcaaatcatggcgtaattagactcaaaagattcgtttcgcaatttacatgcaaactgtgcaattggtttttttccatatttaatgcttcatatatgtattcaaatatttgatgtgacggaattttttaaagtttaaattcaaatatttgatgtgacggaattttttaaagtttgaagggaaataaacacggcctatggcgcaaaattgtactacaaactccctttcgcgagacgagagaatgcaacgccgtgccgctctggcggctggggcccaaccgcgaaaggcaatccatccctctctctccccgtcgcgtaaggccgctataaatcaacccgtcgcgggttgcattgcatcggtcggtctcggtcgctgccgtccctgtgcttggagtagacacgagccggtgacgcgcatatcgtcttctgctcgagatcgatcgatcgatcgcttggttggttgctgcgactgcgagggaggccgcgggtggtgaggaggattgtgcaaaaaaaatggggcgcgcgccgtgctgcgacaaggcgagcgtgaagcgggggccgtggtcgccggaggaggacgagcagctgcggagctacgtccagagccacggcatcggcggcaactggatcgcgctcccgcagaaagcaggtcggtattagtgcactgctcgtcgtcgtcgatgctagccggtgatgaaacgccatgtttgtatgtgaattctgatgtctgcgtgttctcttgtgtgattcagggctcaaccgctgcggcaagagctgcaggctccggtggctcaactacctgaggccggacatcaagcacggcggctacaccgagcaggaggaccacatcatctgctcgctctacaactcgattggaagcaggtgatgatttaagccggagatgaatatttcttttttctttcactgatcctgttctgagttcttgaactgactctgtttgcgtgcgttgccaggtggtccatcatcgcgtcgaagctccccggccggactgacaacgacgtcaagaactactggaacaccaagctcaagaagaaagccatgggcgccgtgcagccgcgcgccgccgcctcggcgccgagccaatgcacgtcgtcagcgatggcgccggcgctctcgccggcctcctcgtccgtcaccagctcgagcggcgacgcctgcttcgccgccgccgccaccaccaccaccaccatgtacccgccaccgacgacgccgccgcagcagcagttcatccgcttcgacgcaccacccgcggcggcggcggcggcatccccgaccgacctcgcgcccgtgccaccaccggccaccgtcacggcggacggcgacggcggctgggcgtccgacgccttgtccctcgacgacgtgttcctcggcgagctcacggccggcgagccgctgttcccttacgccgagctgttcagcggcttcgccggcgcggcgccggacagcaaggccaccttggagctctcggcgtgctacttcccgaacatggcggagatgtgggcggcctccgaccacgcctacgccaagccacagggtctctgcaacaccctgacataggaggacacgctaaaacctccattgaattcttgcgtagctgcgaacgcaacggcgatcgatcgatgcgtcgccacttgctactagcataattcttgctctcttttttttttcaatttttttctcttcttcgagtggcccattctctagctggcgttttctcgtgtattatgagtgatcgattattacatgtaaattaatccatagatgtatctgcactgatccaaattctccagcgattacgcttgtcagttcgtacgcgatccatccgttggcttggctctccggtacaccggacaagttagcgcgtcccgaaaacgatacgcccctgtctgtctccctctcgccgagccgaagtcacttcacatgtacagtgtcacagtgacaaatctgacgagacgaaatgttaggtgaggcacaagaggttcagaagaacaacgtgtgcgagacatggaccagttctcgtgcggcgcgcacgcacagtgtcatggacttgacccgttccggttttcgagggccaatggggaggagaggcgtgtggacggttttctgtcacgttacacgtaggagtattttttttcctctcattttgtttttgatttttgcccggtggcaagttgatcctgatcgtatggcctagggggcctatgggccgtggatcacatggtaattgggtgtcattgtttatttgggtagtctcacttggggcatggatagtgtctctttgtaaagtgattgaagacttcaagtgcacgacaaggacgagcctagttgaccctggaaatggagggaggatcggtttcgaagaagcttccttgacaattgtcatgctcgtgatgggctctatacatatgatcagaggagacctctagttaaaagtctctctaactaattactccaaaaaaaagtctctctaactttgctcattgtgggtttatcaatgtctaggcaaattaaaaccatcaataataacatgttaacagtacttaccttgttggattttactagcttagtgtacgccagggatccacacattttccaggtcttcgtggtcggcatgagaaaatccggcaaaagccaagtagaacaaggccaaaggatgggaaaaagccagtgcaatcctccactgtagttttatttggaaagtagatgaatatggattttttttataaaaaacaaacttttatttacatttgaaaacaaaccccaaaataacttattttacaaataaaccatttgtggagaaaacaacaggcaaggtccccttataacgttgcattttttcattttggtcttttaagacaattgagtcgaaaaatcctatataattcaacctaggttataagcggcatctactacacaacggtaatggcagaggattcacttccaaggtgtacatctatctggtttgtcaagcaaatctttaatttcgttgcttgacattgtcttgcgagggatcaagtttcagttttggttaaaaatatggcaccacattttttttacttgttcttaatcactcttcccctatgcgattgttacttttaaccatagttctctttatatagcgtgtattacccgtcaacaacgaggcgtctgtggtgactttgtcaatctccaagatttaccggtccagccttcaaagatgctcataggggtagggtttgtgtgcgtgcgttcataagggtgagggtgcgtgtgtgttgtgagtgtctgcgttgtattgtgtagttctaaaaaaaacgtaattataaaaagacagtgcaattacgtagtaaatataggtaatttattgtgtaaaaacgatttgataatatattttgaaaaatgtggtatgataaatatagctagttccattaagttttagtccatgtttaacatatatttgtacctcgttcgtgtttccttcatacgcatagcagtaacttatgttttgcattttacaaagcagttacgatgagtaggtcattgtagctcatttacatatggagggttgtgattttgttagtaccttgacattgactcaaagcggcacatctcggcatcaaaggtaggtggcaaaatgcattcgtcaaggacaaatagacaaacatgcattgatcgaatagtgccttcagagccgtggacttctgcgttatcagttcgttgaggtatctctttgatttgacatgagaaatggaccatgtgacagcaagaacgaaatgcatggatgcagtgtcctaactggatagcttgctcctttcagaattaaaagctgctcacttgctagctcagataagtgtcaagacatagtaacagtgtcttcagactctgctttggtttgttgctctaactgaagaattattgctcaccaaacggataagcatatgcacatgtatcagcaaccggcattcggttggaccagtactgacgcagattgcaaacattgttaactcctcgcgatgtctaactgtcttgaaaacgtccttagaaatacttataccggtcaagatcacgagtgtattccaaataattaatttcacctacatcgatgacagaggtgtcaacttaaacatccatagcttcacaatcaagacttcaaatgttttgatgcactagaaagcaaaatcagtaacctttactatgatcatatatcacacacgaatggtgcagctgttagtgtaacaccacgtgttgagatttgagaaagcaggcccaaatcaatgcattattgtagctccatttcactatatcaagtaataatagtaatttatatatatcgtagcagcgaacatgtaccattagattaaaatatgaaaggctcagattcattgcagttctttagtgtgcacttgcaatatcgaagtgcaccctgatctcaatccatcacacatgcacacagactgttcatgtcatcgctgatgctgagttggtcgtgaaatcggacgattccacgacggcaagtacagacacacgtcgattgccggcaaccgcaagcaagtgcagtgcagggtgcaagtgcagcagggcccttggaaacgaagtcggagagagaccatccgcccatgccatgtccatgtccgtgtgtgtgcgctcgctgctgttgtctctttctgagctggtactagtaataaaaactagcccgaggattattgcaagaacaaaaggtaagcagcatcttgcagagcatactatcacccaagctcttaaagctgtcggggaaaagggaccctacgttactcagatacatacggatacgcgatacgtcgatacggggatacgacattttccggatacgacattttccaaaaaaaacaaggatacggggatacgtatatatatatatatatatatattgaaaacacacaaaaaaaattaaggaaattacattaatatatattaaaaatacaaaaaaaataaggagcttttataaagaaaaagcgatataatactagtattagtgtagccgattccatatcaatattttgcaattttcttttctataagcaattcaaattaacaagcgattaagatcccaaaaactacatattaatagttcaatcatataactcttgatgactttgcatcaacattagcagccgcactaaccagtaaccactagaattgctagatgcactagcacttgtatctaacaaaaacgatagtactttgtcctaagaaaaacaagaaaggcccaacagattcaagcaagaaatgttcaaagagcatggatttacccggccaggcactcaagcaaacaatgaaaaagctccttgctttgatgttgccagcggccagcctgccaccggcctccgctatgcccggcaacggtcgtggcctccgaggttggcggcgaaggagatgcgccaccgcccagccgcggcagctcttccgtctcgcggagaggacgttgtggcccgcgcacggcaaccaagcgcgctgctgcagccgcgtctctggtggcatcgccgggcacgcacgagagacagccatcaggatgggagcgtatgaggcggcgcatccctgatgcgagcgaagacggaggcacgtcgcggccgtatcgctggcatatctcgatttattttgtttttttaaatcgcgaatatgtgggatacgtacggaatatgtatcggaacatatccgatatgtatccgtatccgttgccgtatcagatacggatacgtcgctctgtagccgtaccaggataacgttgagttagggacctaccttcagaaaatatatattcatggatgctagtacaatccagagtagtgacttcaaaagctgaagatctgagctttttgtaccgctaacaaactcagttttttttaaggctgtgtttagtttctgagattagggagaagtttggggaaagttggtaaattagaaaaaaagttgggagtttatttaagtatgaaagttttgaatgtgatgtgatggaaagttgagagtttgggggaagtttggtgtgaactaaatagggcctaatttgttttagcatcccgtgaaacatttttgctatttctataggccttgtttagttccaaactttttcttcaaactttcaacttttccatcacatcgttccaatttcaaccaaacttctaattttggcgtgaaccaaacttctcaattttctgtgtaacttttggtctcggttattggatatcctgatgcatcatgcattgactgtacatgtcataggtcttctattcattactagtacaatatacaggttttttttgggataaacttggatgctttgttcagcatctgccttttctgtcttgactcttggtaccgcttgtgaacatgtcgacatgttgttcctgctgtttttttttccttttcattttgaggcatgcgtcactaacttatgttcgagtccatttttcagcatt

SEQ ID NO: 4 - DNA, 870 bp, Oryza sativa
atggggcgcgcgccgtgctgcgacaaggcgagcgtgaagcgggggccgtggtcgccggaggaggacgagcagctgcggagctacgtccagagccacggcatcggcggcaactggatcgcgctcccgcagaaagcagggctcaaccgctgcggcaagagctgcaggctccggtggctcaactacctgaggccggacatcaagcacggcggctacaccgagcaggaggaccacatcatctgctcgctctacaactcgattggaagcaggtggtccatcatcgcgtcgaagctccccggccggactgacaacgacgtcaagaactactggaacaccaagctcaagaagaaagccatgggcgccgtgcagccgcgcgccgccgcctcggcgccgagccaatgcacgtcgtcagcgatggcgccggcgctctcgccggcctcctcgtccgtcaccagctcgagcggcgacgcctgcttcgccgccgccgccaccaccaccaccaccatgtacccgccaccgacgacgccgccgcagcagcagttcatccgcttcgacgcaccacccgcggcggcggcggcggcatccccgaccgacctcgcgcccgtgccaccaccggccaccgtcacggcggacggcgacggcggctgggcgtccgacgccttgtccctcgacgacgtgttcctcggcgagctcacggccggcgagccgctgttcccttacgccgagctgttcagcggcttcgccggcgcggcgccggacagcaaggccaccttggagctctcggcgtgctacttcccgaacatggcggagatgtgggcggcctccgaccacgcctacgccaagccacagggtctctgcaacaccctgacatag

SEQ ID NO: 5 - protein, 289 aa, Oryza sativa
MGRAPCCDKASVKRGPWSPEEDEQLRSYVQSHGIGGNWIALPQKAGLNRCGKSCRLRWLNYLRPDIKHGGYTEQEDHIICSLYNSIGSRWSIIASKLPGRTDNDVKNYWNTKLKKKAMGAVQPRAAASAPSQCTSSAMAPALSPASSSVTSSSGDACFAAAATTTTTMYPPPTTPPQQQFIRFDAPPAAAAAASPTDLAPVPPPATVTADGDGGWASDALSLDDVFLGELTAGEPLFPYAELFSGFAGAAPDSKATLELSACYFPNMAEMWAASDHAYAKPQGLCNTLT

SEQ ID NO: 6 - protein, 289 aa, Sorghum bicolor
MGRAPCCDKASVKRGPWSPEEDEQLRSYVQRNGIGGNWIALPQKAGLNRCGKSCRLRWLNYLRPDIKHGGYTEEEDRIIWSLYSSIGSRWSIIASKLPGRTDNDVKNYWNTKLKKKAMAMAAAAGLSASSSGGGAFAAAAAPATPTPPALSPASSSVTSSSGDVRFGAAYTEPQPQPQHPGGLIRFDAPRTELAPVPPAVGAQLDGAWSTPVAPALDGGDVFLPELIGGGEQLFPYGGDFFGGLLQDSRALEQLSACYFPNMAEIWGATAAVAAPDGNCKPPGLCNTLT

SEQ ID NO: 7 - protein, 262 aa, Zea mays
MGRAPCCDKASVKRGPWSPEEDEQLRSYVQLNGIGGNWIALPQKAGLNRCGKSCRLRWLNYLRPNIKHGGYTDEEDRIIWSLYSSIGSRWSIIASKLPGRTDNDVKNYWNTKLKKKAMAASSSGAAFAAPATPALSPASSCVTSSSSGDVRLGAAYTEPPPPRQHAGLVRSDAPRTELAPVPAVAHLDGACWPAAAALDVLLPELGGEQLFPYGYGDFFGGLQDRALEQQLSSCYFPNVAEIWGAAAVAPDGKPPGLCNRLT

SEQ ID NO: 8 - protein, 259 aa, Vitis vinifera
MGRAPCCDKANVKRGPWSPEEDATLRNYVQTHGIGGNWIALPRKAGLRRCGKSCRLRWLNYLRPDIKHGGFTEEEDKVICAIYNQMGSRWSVIASQLPGRTDNDVKNYWNTKLKKKLAKFHSNGNAANNPNPFSSAPPPTAAPMPVTEAYRHGFPPLLTQGYTGTSNNMGVSEFGASSNNGHNVFISDQGGLSTDHATGISYVSYLPGTRGGEDDGVLMDFGMGNLPYDIISGFWFQEKDSTEVTPGHDQSRFLLIDGN

SEQ ID NO: 9 - protein, 302 aa, Populus trichocarps
MGRAPCCDKANVKRGPWSPEEDATLKSYLETHGTGGNWIALPQKAGLKRCGKSCRLRWLNYLRPDIKHGGFTEEEDNIICTLYSQMGSRWSLIAAQLPGRTDNDVKNYWNTKLKKKILAGKISLTIKNNPINAPVNVANIPAIPCSTSPILYVPKAETESSVTFSDHYSLTQISGTLPALSDIGYEPIINSTAQNLSPNQFQFSSFPGVIDMSEFSMNSTHIVSPSQEGSSISDSSSLAMDNKGLSVPSTNGGLEDVGIFMDSEPGFPSDFFNGLLLQDKASEVATNCYQYFAGFGYVDIKP

SEQ ID NO: 10 - protein, 348 aa, Malus x domestica
MGRAPCCDKANVKKGPWSPEEDSKLKEYIEKYGTGGNWIALPQKAGLKRCGKSCRLRWLNYLRPNIKHGEFSDEEDRIICSLFASIGSRWSVIAAQLPGRTDNDIKNYWNTKLKKKLMGMHMGPPRPHNHHKNLLKPPPFPSSSHHNYQNQPLIPSEPLSSLYKDLSNYNRSFLGFEAPVPLPPQVSLTSNNFSNISTNSSIFQTLNYPAGVKENNNNNTLLVFGSEGSCSTSSDGSCNNQISYDYCSRSEINNINQEEMGFDHQGFMMLNYGNQWTERPNGFYFGSENNTLEFTDLDEDVKQQLISTRSKNNYNNNTIINESSKSNSFLFNVNESKTEDDEKVMYFY

SEQ ID NO: 11 - protein, 193 aa, Glycine max
MGRAPCCDKANVKKGPWSPEEDAKLKSYIEQHGTGGNWIALPQKIGLKRCGKSCRLKWLNYLRPNIKHGGFSEEEDNIICSLYVSIGSRWSIIAAQLPGRTDNDIKNYWNTRLKKKLLGKQRKEQQAQARRVSNNQQKQDVKRETENLIVASEVMINQVPYWSSSEFSSVPMPVTNASSFQHYGLNNQTSLRA

SEQ ID NO: 12 - protein, 313 aa, Daucus carota
MGRAPCCDKASVKRGPWSPEEDQKLKDYIEKHGTGGNWIALPQKAGLRRCGKSCRLRWLNYLRPNIKHGEFSDDEDRIICTLFANIGSRWSIIAGQLPGRTDNDIKNYWNTKLKKKLLMSSLMLPNPFVRPPNNINNYQQYLLSSPSPSSYSSPLPPTLRLCDNNYPTLGASRSFTSEGFSNISTNLFNIQPQDQGLGPMQNYQEKESPLIMFGGAGDDQQAASSSSYDEHFMMFSNCGMNNISDQQKQVNLGVSSGDHEIVSNVLDQYNMSCVEEIKQLISTTNLSCNSNLSFFVDENKTVRSEEEKVLMYY

SEQ ID NO: 13 - protein, 333 aa, Arabidopsis thaliana
MGRAPCCDKANVKKGPWSPEEDVKLKDYIDKYGTGGNWIALPQKIGLKRCGKSCRLRWLNYLRPNIKHGGFSEEEDRIILSLYISIGSRWSIIAAQLPGRTDNDIKNYWNTKLKKKLLGRQKQMNRQDSITDSTENNLSNNNNNKSPQNLSNSALERLQLHMQLQNLQSPFSSFYNNPILWPKLHPLLQSTTTNQNPKLASQESFHPLGVNVDHQHNNTKLAQINNGASSLYSENVEQSQNPAHEFQPNFGFSQDLRLDNHNMDFMNRGVSKELFQVGNEFELTNGSSWWSEEVELERKTTSSSSWGSASVLDQTTEGMVMLQDYAQMSYHSV

SEQ ID NO: 14 - DNA, 870 bp, Sorghum bicolor
atggggcgcgcgccgtgctgcgacaaggcgagcgtgaagcggggaccctggtcgcctgaggaggacgagcagctgcggagctacgtccagcgcaacggcatcggcggcaactggatcgccctgccgcagaaagcaggcctgaaccggtgcggcaagagctgccggctgcggtggctcaactacctgcggccggacatcaagcacgggggctacaccgaggaggaggaccggatcatctggtcgctctacagctccatcggcagcaggtggtccatcatcgcgtccaagctcccgggccggaccgacaacgacgtcaagaactactggaacaccaagctcaagaagaaggccatggccatggcggcggcggccggcctcagcgccagtagcagcggaggaggagccttcgcggcggcggcggcgcccgccacgccgacgccgccggcgctctcgcccgcctcctcgtccgtcaccagctccagcggcgacgtgcgcttcggcgcggcgtacacggaaccacaaccgcagccgcagcaccccggcgggctcatacgcttcgacgcgccgcggacggagctcgcgcccgtgccgccggcagtgggagcgcagctggacggcgcctggtcgacgcccgtggcgccggcgctggacggcggggacgtgttcctgcccgagctcatcggcggcggcgagcagctgttcccgtacggcggcgacttcttcggcgggctgctgcaggacagcagggccctggagcagctctcggcgtgctacttccccaacatggccgagatatggggcgccaccgctgccgtcgccgcccccgacggcaactgcaagccgccgggtctctgcaacaccctgacatag

SEQ ID NO: 15 - DNA, 789 bp, Zea mays
atgggccgcgcgccgtgctgcgacaaggcgagcgtgaagcgggggccctggtcgcccgaggaggacgagcagctgcggagctacgtccagctgaacggcatcggcggcaactggatcgccctgccgcagaaagcagggctgaaccggtgtggcaagagttgccggctgcggtggctcaactacctgcggccgaacatcaagcacggcggctacaccgacgaggaggaccggatcatttggtcgctctacagctccatcggcagcaggtggtcgatcatcgcgtccaagctccccggccggaccgataacgacgtcaagaactactggaacaccaagctcaagaagaaggccatggcggccagtagcagcggagcagccttcgcggcgcccgccacgccggcgctctcgcccgcctcctcgtgcgtcaccagctcctccagcggcgacgtgcgcctcggcgcagcgtacacggagccgccgccgcctcggcagcacgcggggctcgtacgctctgacgctccgcggacggagctcgcgcccgtgccggcagtcgcgcacctggacggcgcctgctggccggccgcggcggcgctggacgtgctcctgcccgagctcggcggcgagcagctgttcccgtacgggtacggcgacttcttcggcgggctgcaggacagggccctggagcagcagctctcgtcgtgctacttccccaacgtggccgagatatggggcgccgctgccgtcgcccccgacggcaagccgccgggtctctgcaacaggctgacgtag

SEQ ID NO: 16 - DNA, 909 bp, Populus trichocarps
atggggagagctccttgttgtgacaaagcaaacgtgaaaagaggaccatggtctcctgaggaagatgccactctcaaaagctaccttgagactcatggcactggtggtaactggattgctttgcctcaaaaagctggccttaagcgatgcggcaagagctgccggttacgatggcttaattatctcagaccagacatcaaacatggaggctttactgaagaagaagacaacatcatatgcactctctatagtcaaatgggaagcagatggtctctcatagctgctcaactgcctggaagaactgacaacgatgtgaaaaattactggaacaccaaattgaaaaagaagattcttgcgggaaaaataagcctcacaatcaagaacaaccctatcaatgcccccgttaacgttgctaatattcctgccattccctgctcaacatctcctattctttatgtccctaaagctgaaaccgaaagttctgtcaccttctccgatcattattcattaactcaaatttcaggaacattgcctgccttatctgatattggttatgaacctattatcaatagcactgcccagaacttgagtccaaaccaattccaattttctagtttccctggggtcattgacatgtctgaattttcaatgaacagtactcacattgtttctccatctcaagaaggttcgagcatttcagattcctcttcacttgctatggataacaagggtctctcagtgcctagtactaacggtggtcttgaggatgttgggatatttatggattccgaacctggttttcctagtgattttttcaatggccttttgcttcaagacaaagctagtgaagtggccactaattgctaccaatattttgctggttttggctatgttgatattaagccgtaa

SEQ ID NO: 17 - DNA, 1047 bp, Malus x domestica
atggggagggctccttgttgtgacaaagcaaacgtgaagaagggaccatggtcaccagaagaagattcaaagctaaaagagtacatagagaagtatgggactggtgggaattggattgctctcccacagaaagctggtctgaagagatgtgggaaaagctgcagattgagatggcttaactatctgaggccaaacatcaaacatggagaattctctgatgaggaagacaggataatatgcagcctctttgctagtattggaagcaggtggtcagttatagctgctcagctgccaggcaggactgacaacgatatcaagaactactggaacaccaagctcaagaagaagctcatggggatgcatatgggtcctcctcgacctcacaaccaccataaaaatttactaaagcctcccccatttccttcttcctctcatcacaattaccaaaaccaaccattaattccatctgaacctctatcgtcgctgtacaaagacctaagcaattacaacagatctttcttagggtttgaagcgccagtgccattgccaccacaagtttcgctgacgtccaataatttctcaaacatttccaccaactcttctatttttcaaaccctaaattacccagctggagtgaaggaaaataacaataataataccctcctcgtgtttggaagtgaagggagttgcagtacttcgtctgatggaagctgtaataatcagatcagctatgactactgcagcagatcagagattaataacatcaaccaagaagaaatgggttttgatcatcagggcttcatgatgcttaactatggcaaccaatggaccgaaaggccaaatgggttttattttggatcagaaaacaacacattagaatttactgatctggacgaagatgttaagcagcagctgattagtactagaagtaaaaataattataataataatactattataaatgagtcctctaagtctaacagctttttattcaatgttaatgaaagcaagacagaagatgatgagaaggtcatgtatttctactga

SEQ ID NO: 18 - DNA, 582 bp, Glycine max
atgggaagagctccttgctgtgacaaagccaacgtgaagaaaggtccatggtcacctgaagaagatgccaaactcaagtcttatattgagcagcatggcactggtggaaactggattgccttgccccagaaaataggccttaagaggtgtgggaaaagctgtcgtcttaaatggctgaattatcttcgcccaaacatcaaacatgggggtttctcagaagaagaagacaacatcatctgcagcctctatgttagtattggaagcaggtggtcaattatagcagcacagttacccgggcgcacagacaatgatataaagaactattggaacactaggctgaagaagaagctccttggcaaacaaaggaaggagcaacaggctcaagctcgccgagtcagcaacaaccagcagaagcaagatgtcaaaagagaaaccgagaatttgatagtggcttctgaagtcatgatcaatcaagttccctattggtcttcttcagagttttcttctgtgcctatgccagtgaccaatgcttcttcatttcaacactatggtctgaacaatcaaacatctctcagagcatga

SEQ ID NO: 19 - DNA, 942 bp, Daucus carota
atggggagagctccttgctgtgacaaggcaagtgtaaagagagggccatggtcacctgaagaagatcagaagctaaaagactatatagagaagcatgggactggaggaaactggattgctctaccacaaaaggctggtcttagaagatgtgggaaaagctgcagattaagatggctcaactatctcaggcctaacattaaacatggagaattttcagatgatgaagataggatcatatgcaccctctttgccaatattgggagcaggtggtcaataatagcaggtcagttaccagggaggactgacaatgatatcaagaactactggaacaccaagctcaagaagaaactcctcatgtcttcattaatgctccctaatccttttgttaggcctcctaataatattaataactaccaacaatacctattatcatcaccatctccttcttcatattcctccccattgcctccaacattgagactttgtgacaacaattacccaaccctaggtgcttctaggtcattcacaagtgagggcttctcaaatatttcaacaaacctctttaatatccagcctcaagatcagggtttgggtcccatgcaaaattatcaagagaaagaatcacctctcattatgtttggaggggcaggtgatgatcagcaagctgctagctctagttcttatgatgaacatttcatgatgttctccaactgtgggatgaataatatttctgatcaacagaagcaagtaaatcttggtgtgtctagtggtgatcatgagattgttagcaatgtactggatcagtataacatgagctgtgttgaggagattaagcaactgattagcactactaatctgtcatgtaattctaatctgagtttctttgttgatgaaaacaagacagtgagaagtgaggaggagaaagtgttgatgtactactga

SEQ ID NO: 20 - DNA, 1002 bp, Arabidopsis thaliana
atgggaagagctccatgctgcgacaaggcaaacgtgaagaaaggaccatggtcaccggaagaagatgtgaagctcaaggattacatcgacaaatatggcactggtggcaactggatcgcactgcctcagaaaattgggctgaagagatgtggtaagagttgcagactgagatggcttaattacttaagaccaaacatcaaacatggtggtttttctgaggaagaagatagaatcatcttgagtctctacattagcattggaagccggtggtccataattgcagctcagcttcctggaaggactgacaatgatatcaagaattattggaacacaaaactgaagaagaaacttctaggaagacagaaacaaatgaatcgtcaagactccataaccgattctactgagaacaacctcagcaacaataacaacaataagagtcctcagaatctgagcaattcggcactggagaggctccagcttcacatgcagcttcagaatctacagagccctttctctagtttctacaacaaccctatcttgtggcccaagcttcatccattgctccagagcactacaactaatcaaaaccctaagcttgcatctcaagaaagcttccaccctttaggagttaacgttgatcatcagcacaacaataccaagctagctcagataaacaatggagcctcttctctctattcggagaacgtagagcaatcccaaaaccctgctcatgaatttcaacctaatttcggtttttcacaggaccttcgattagataatcataacatggactttatgaacagaggggtttctaaagaactgtttcaagtgggcaacgagtttgagctaacgaacggttcgagttggtggtcagaggaagtggaactagagaggaaaacgacgtcgtcgagttcttgggggtcagcttctgtgcttgatcagacaactgagggaatggttatgcttcaagattacgctcagatgagctaccacagtgtttaa

SEQ ID NO: 21 - DNA, 29 bp, Artificial
  PCR primer
tggtgaggaggattgtgcaaggatccgcg

SEQ ID NO: 22 - DNA, 29 bp, Artificial
  PCR primer
ccggaattcttgcacaatcctcctcacca

SEQ ID NO: 23 - DNA, 21 bp, Artificial
  PCR primer
cgtcaagaactactggaacac

SEQ ID NO: 24 - DNA, 20 bp, Artificial
  PCR primer
ccatgttcgggaagtagcac

SEQ ID NO: 25 - DNA, 25 bp, Artificial
  PCR primer
cgcggatccatggggcgcgcgccgt

SEQ ID NO: 26 - DNA, 34 bp, Artificial
  PCR primer
cccaagctttgtcagggtgttgcagagaccctgt

SEQ ID NO: 27 - DNA, 26 bp, Artificial
  PCR primer
cacctgcggtgaatggaagacgtttg

SEQ ID NO: 28 - DNA, 28 bp, Artificial
  PCR primer
tgctcaaagcagaagagatctgaatgag

SEQ ID NO: 29 - DNA, 29 bp, Artificial
  PCR primer
caccgacggaggaagtagtgtggaaccat

SEQ ID NO: 30 - DNA, 17 bp, Artificial
  PCR primer
tggtggtagggtgcggc

SEQ ID NO: 31 - DNA, 27 bp, Artificial
  PCR primer
cacccagatcaaatgtcaaaaggggcg

SEQ ID NO: 32 - DNA, 22 bp, Artificial
  PCR primer
cttgcctgccgagctatcaacc

SEQ ID NO: 33 - DNA, 17 bp, Artificial
  PCR primer
atggggcgcgcgccgtg

SEQ ID NO: 34 - DNA, 24 bp, Artificial
  PCR primer
ctatgtcagggtgttgcagagacc

SEQ ID NO: 35 - DNA, 2144 bp, Oryza sativa
gagaaactaccagaatgcccgtatgttgcaacacgattttaattaattaaagaaaatatcattaatctattattataatcttttaattcaattgatgtagatatttgttcctgacttgtggatttttttagtctcacactctcccttttttttctttctcatgggagtggtcgatatgtgcagcgtctaacaccacatgtcctttttcttcgaaaaaagagttctcccttataacttttataaggagtatagatagtttgcattccttgcttgctgctttttttatagatataatttgcatttccttgttgcactgatcattagctcattagttcatcccatgaggacatgatgacagtatgacactacccagaatgattatttttcacttaatatttggagatttatctcgacgtcactgggtggcatataagggatgcgacaaacactccatatcaatcgaggaatttcctctcgtcttgaattatgactgatcatgacttgtcattagagaagttaaagaaaaagatgggacttatcaggagagactaaaaaaagagtagccctacgagtagtttagtgtgttttgccatactaccatgtgatggtgatgtcaaaaacatggggctcgcgtaaccgcaaaactaaggctgtcccagacacactaatcaaaagcattcgaatttagaactcctataatttgttagtttgtttaatcactctttgtgtctcatgatgattcatgcttgcataattgcatacacttctcgactctaagcttggaccacacaacccgtacataggacgaaaccgaaagcatcacccaaaaaaaaaaaaactaatgtgcccctctttagcatgtcagaataatatggaaaaggcgtcgagcagaaacaaatatgtcaagagtttgacaaacctgacaactgccaatgcagccaatgagccaagcagcaacttgttgatggctggtaggctttcccattatattctggcaaaaactttgcttccatttttctattgacgacgatgagttcgccgattccggccgggccggctcagcttttctccttcggctagcgtttggacgcacacttttgagagaatcgaaacacggagagaagtgctccgatgcatgacacctccttttccacgtacttaacgccaaagggaaacaatgatatgcagtgaacacaaagggattgaccctgcccaggatcatcaatacacgttgaggcttcttcgtgtgtgaaagcatggctaactcgtctggtcaaaggcaaaattgctgctatcctaaaacaaatagtactaggataaataaatgaaagggctaatataacactcgcacacacgcgagacgacaaaatggagagctagcacacacgatagggacggctcgacggtaggggaggaggaataatgcaggcaggatgttactatggccacgtttagttggttagtgtgacaaaaaatttttaaaagtatacagacatacataatatatatttaaagtattaaaggtagactaataacaaaataaattacagatttcacatataaactgtgaggtgaatttattaagtttaattaatccttcattagcaaatgtttactgtagcatcacattgtcaaatcatggcgtaattagactcaaaagattcgtttcgcaatttacatgcaaactgtgcaattggtttttttccatatttaatgcttcatatatgtattcaaatatttgatgtgacggaattttttaaagtttaaattcaaatatttgatgtgacggaattttttaaagtttgaagggaaataaacacggcctatggcgcaaaattgtactacaaactccctttcgcgagacgagagaatgcaacgccgtgccgctctggcggctggggcccaaccgcgaaaggcaatccatccctctctctccccgtcgcgtaaggccgctataaatcaacccgtcgcgggttgcattgcatcggtcggtctcggtcgctgccgtccctgtgcttggagtagacacgagccggtgacgcgcatatcgtcttctgctcgagatcgatcgatcgatcgcttggttggttgctgcgactgcgagggaggccgcgggtggtgaggaggattgtgcaaaaaaaatg

(19) United States
(12) Patent Application Publication
(10) Pub. No.: US 2015/0218578 A1
(43) Pub. Date: Aug. 6, 2015

(54) METHODS OF INCREASING TOLERANCE TO HEAT STRESS AND AMINO ACID CONTENT OF PLANTS

(71) Applicant: University of Guelph, Guelph (CA)

(72) Inventors: Steven Rothstein, Guelph (CA); Ashraf El-kereamy, Guelph (CA); Yongmei Bi, Guelph (CA)

(73) Assignee: University of Guelph, Guelph, ON (CA)

(21) Appl. No.: 14/384,929

(22) PCT Filed: Mar. 13, 2013
(86) PCT No.: PCT/IB2013/051975

§ 371 Date: Sep. 12, 2014

Related U.S. Application Data
(60) Provisional application No. 61/610,288, filed on Mar. 13, 2012.

(51) Int. Cl. C12N 15/82

(52) U.S. Cl. CPC C12N 15/8251; C12N 15/8205

(57) ABSTRACT

The present invention provides methods of increasing tolerance to high temperature or heat stress in a plant, plant part, or plant cell, the method comprising introducing one or more nucleic acids encoding (i) a glutamine synthetase 1;2 (GS 1;2), (ii) a glutamate decarboxylase 3 (GAD3), (iii) a class I glutamine amidotransferase (GAT1), (iv) a MYB55 polypeptide or any combination thereof into a plant, plant part or plant cell. Also provided are methods of increasing amino acid content in a plant, plant part, or plant cell, the method comprising introducing one or more nucleic acids encoding (i) a GS1;2, (ii) a GAD3, (iii) a GAT1, (iv) a MYB55 polypeptide or any combination thereof into a plant, plant part or plant cell.

### RELATED APPLICATIONS

This application is a 35 U.S.C. §371 national stage application of International Application No. PCT/IB2013/051975, filed Mar. 13, 2013, which claims the benefit of priority from U.S. Provisional Application No. 61/610,288, filed Mar. 13, 2012, the contents of each of which are incorporated herein by reference in their entireties. The above-referenced International Application was published as International Publication No. WO 2013/136273 A2 on Sep. 19, 2013.

### STATEMENT REGARDING THE ELECTRONIC FILING OF A SEQUENCE LISTING

A sequence listing in ASCII text format, submitted under 37 C.F.R. §1.821, entitled 9973-70TS_ST25.txt, 60,419 bytes in size, generated on Mar. 30, 2015, and filed electronically via EFS-Web, is provided in lieu of a paper copy.

### FIELD OF THE INVENTION

The present invention relates to methods of increasing tolerance to heat stress or high temperature and methods of increasing amino acid content of a plant, plant part or plant cell.

### BACKGROUND OF THE INVENTION

Plants are subject to various stress conditions that may adversely affect their productivity. For instance, heat stress may adversely affect various aspects of a plant's growth and development, including, but not limited to, fertility, seed germination, coleoptile growth, grain filling and/or fruit colour. See, e.g., Ashraf et al., ENVIRON. EXP. BOT. 34:275 (1994); Endo et al., PLANT CELL PHYSIOL. 50:1911 (2009); Jagadish et al., J. EXP. BOT. 61:143 (2010); Kolupaev et al., RUSSIAN J. PLANT PHYSIOL. 52:199 (2005); Lin et al., J. AGRIC. FOOD CHEM. 58:10545 (2010); Lin-Wang et al., PLANT CELL ENVIRON. 34(7):1176 (2011); Morita et al., ANN. BOT. 95:695 (2005)). Rice, which provides food to approximately half the world's population, may be particularly susceptible to heat stress. See Peng et al., PROC. NATL. ACAD. SCI. USA 101:9971 (2004) (describing decreased rice yields at the International Rice Institute in response to the global increase in nighttime temperatures between 1992 and 2003).

Although most heat response studies have focused on heat shock transcription factors and heat shock proteins, heat tolerance is a complex process that involves numerous genes, pathways and systems. Indeed, a variety of proteins, molecules and pathways have been shown to play a role in heat stress responses in cotton, wheat, corn and other plants.

MYB transcription factors regulate numerous processes during the plant life cycle and are classified into three major groups based upon the number of adjacent repeats in their binding domains: R1R2R3-MYB, R2R3-MYB, and R1-MY18. Most plant MYB transcription factors are of the R2R3 type, which are involved in a wide range of physiological responses such as regulation of the isopropanoid and flavonoid pathways, control of the cell cycle, root growth, and various defense and stress responses. Du et al., BIOCHEM. (MOSC) 74:1 (2009); Jin and Martin, PLANT MOL. BIOL. 41:577 (1999); Lee et al., MOL. PLANT MICROBE INTERACT. 14:527 (2001); Lin-Wang et al., BMC PLANT BIOL. 10:50 (2010); Mellway et al., PLANT PHYSIOL. 150:924 (2009); Mu et al., CELL RES. 19:1291 (2009); Raffaele et al., PLANT CELL 20:752 (2008); Stracke et al., CURR. OPIN. PLANT BIOL. 4:447 (2001); Sugimoto et al., PLANT CELL 12:2511 (2000); Yang and Klessig, PROC. NATL. ACAD. SCI. USA 93:14972 (1996)). Despite the large number of genes in the MYB family, the Arabidopsis MYB68 gene is the only member proposed to have a role in Arabidopsis heat tolerance. Feng et al., PLANT SCI. 167:1099 (2004) (describing a mutant MYB68 plant with reduced growth and higher lignin levels in the roots when grown at a high temperature).

### SUMMARY OF THE INVENTION

As one aspect, the invention provides a method of increasing tolerance to heat stress or high temperature in a transgenic plant, plant part or plant cell, the method comprising introducing one or more isolated nucleic acids encoding (i) a glutamine synthetase 1;2 (GS1;2), (ii) a glutamate decarboxylase 3 (GAD3), (iii) a class I glutamine amidotransferase (GAT1), (iv) a MYB55 polypeptide or any combination thereof into a plant, plant part or plant cell to produce a transgenic plant, plant part or plant cell that expresses the one or more isolated nucleic acids to produce GS1;2, GAD3, GAT1, MYB55, or any combination thereof, thereby resulting in an increased tolerance to heat stress or high temperature in the transgenic plant, plant part or plant cell as compared with a control.

In representative embodiments, the method comprises: (a) introducing the one or more isolated nucleic acids into a plant cell to produce a transgenic plant cell; and (b) regenerating a transgenic plant from the transgenic plant cell of (a), wherein the transgenic plant comprises in its genome the one or more isolated nucleic acids and has increased tolerance to heat stress or high temperature.

In additional embodiments, the method comprises: (a) introducing the one or more isolated nucleic acids into a plant cell to produce a transgenic plant cell; (b) regenerating a transgenic plant from the transgenic plant cell of (a), wherein the transgenic plant comprises in its genome the one or more isolated nucleic acids; and (c) selecting from a plurality of the transgenic plants of (b) a transgenic plant having increased tolerance to heat stress or high temperature.

As a further aspect, the invention provides a method of increasing amino acid content in a transgenic plant, plant part or plant cell, the method comprising introducing an isolated nucleic acid encoding a MYB55 polypeptide into a plant, plant part or plant cell to produce a transgenic plant, plant part or plant cell that expresses the isolated nucleic acid to produce the MYB55 polypeptide resulting in an increased amino acid content in the transgenic plant, plant part or plant cell as compared with a control.

In representative embodiments, the method comprises: (a) introducing the isolated nucleic acid into a plant cell to produce a transgenic plant cell; and (b) regenerating a transgenic plant from the transgenic plant cell of (a), wherein the transgenic plant comprises in its genome the isolated nucleic acid and has increased amino acid content.

In additional embodiments, the method comprises: (a) introducing the isolated nucleic acid into a plant cell to produce a transgenic plant cell; (b) regenerating a transgenic plant from the transgenic plant cell of (a), wherein the transgenic plant comprises in its genome the isolated nucleic acid; and (c) selecting from a plurality of the transgenic plants of (b) a transgenic plant having increased amino acid content.

The invention also provides a method of obtaining a progeny plant derived from a transgenic plant of the invention, wherein the progeny plant comprises in its genome an isolated nucleic acid of the invention and has increased tolerance to high temperature or heat stress and/or an increased amino acid content.

As yet another aspect, the invention encompasses a transgenic plant, plant part or plant cell produced by a method of the invention, optionally wherein the transgenic plant, plant part or plant cell has increased tolerance to heat stress or high temperature and/or an increased amino acid content.

These and other aspects of the invention are set forth in more detail in the following description of the invention.

### BRIEF DESCRIPTION OF THE DRAWINGS

FIG. 1A depicts an unrooted phylogenetic tree showing the similarity between Oryza sativa MYB55 (OsMYB55) and several of its homologues in other species.

FIGS. 1B-1F show various sequences described herein. FIG. 1B depicts the portion of the OsMYB55 promoter sequence (SEQ ID NO:1) that was used to drive expression of beta-glucuronidase (GUS) in the expression assays described in Example 3. FIG. 1C depicts the OsMYB55 promoter sequence (SEQ ID NO:2) and the adjoining 5′ untranslated region (UTR). FIG. 1D depicts the OsMYB55 gene sequence (SEQ ID NO:3), including the 5′ UTR, the promoter sequence, the coding region and the 3′ UTR. FIG. 1E depicts the nucleotide sequence of the OsMYB55 cDNA (SEQ ID NO:4). FIG. 1F depicts the amino acid sequence of the OsMYB55 protein (SEQ ID NO:5). Nucleotides residing in a promoter sequence are underlined. Nucleotides residing in a coding sequence are shown as uppercase letters. Amino acids residing in a DNA binding region are shown as bold, italicized letters.

FIG. 2A shows the OsMYB55 promoter sequence (SEQ ID NO:35), with cis-acting regulatory elements (CAREs) and transcription factor binding sites (TFBS) highlighted therein. “MeJa” refers to CAREs involved in MeJa responsiveness. “HSE” (SEQ ID NOs:36 and 37) refers to CAREs involved in heat stress responsiveness. “ABRE” refers to CAREs involved in abscisic acid responsiveness. “TCA” (SEQ ID NO:38) refers to CAREs involved in salicylic acid responsiveness. “LTR” refers to CAREs involved in low temperature responsiveness. “Skn-1” refers to CAREs involved in endosperm expression. “GCC Box” refers to binding sites for activating protein-2 (AP-2) transcription factors. “MBS box” refers to binding sites for MYB transcription factors. “W box” refers to binding sites for WRKY transcription factors. “DOF box” refers to binding sites for DNA-binding with one finger (DOF) transcription factors. The ATG start codon of the OsMYB55 coding sequence is indicated with asterisks.

FIG. 2B is a diagram that graphically depicts the location of potential CAREs in the OsMYB55 promoter region. “MeJa” refers to CAREs involved in MeJa responsiveness. “HSE” (SEQ ID NOs:36 and 37) refers to CAREs involved in heat stress responsiveness. “ABRE” refers to CAREs involved in abscisic acid responsiveness. The numbers below the diagram indicate the positions of the CAREs relative to the ATG initiation codon.

FIG. 3 depicts the relative gene expression levels of OsMSB55 at various stages in the life cycle of wild-type rice plants grown under normal growth conditions.

FIG. 4 is a graph showing the relative OsMYB55 transcript levels (mean±standard deviation, n=3) of leaves taken from wild-type rice plants grown under normal growth conditions for four weeks and then exposed to 45° C. for 0, 1, 6 or 24 hours.

FIG. 5 shows cross sections of the leaf sheaths (I, II), leaf blade (III) and roots (IV) taken from rice plants expressing GUS under the control of a 2134 base pair fragment (SEQ ID NO:1) of the OsMYB55 promoter region (OsMYB55promoter-GUS) that were grown under normal growth conditions for 4 weeks and then exposed to 29° C. (left) or to 45° C. (right) for 24 hours. Plant tissues were immersed in a solution containing 1 mg/ml 5-bromo-4-chloro-3-indolyl β-G-glucuronide (X-Gluc; Biosynth, Itasca, Ill.) to stain GUS protein, and cross sections were taken and visualized using a light microscope.

FIG. 6 is a graph showing OsMYB55 transcript levels in the leaves of wild-type rice plants (WT) and transgenic rice plants overexpressing OsMYB55 (OsMYB55-4; OsMYB55-11) grown under normal growth conditions for four weeks.

FIG. 7A shows seeds from wild-type rice plants (WT) and transgenic rice plants overexpressing OsMYB55 (OsMYB55-4; OsMYB55-11) following germination and four days of growth at 28° C. or 39° C.

FIG. 7B is a graph showing the coleoptile lengths (mean±standard deviation, n=3) of wild-type rice plants (WT) and transgenic rice plants overexpressing OsMYB55 following germination and four days of growth at 28° C. (Control) or 39° C. (High temperature). Each replicate consisted of 25 seedlings.

FIG. 8A shows wild-type rice plants (WT) and transgenic rice plants overexpressing OsMYB55 (55:4; 55:11) following germination under normal growth conditions and four weeks of growth in Turface® MVP® (PROFILE Products, LLC, Buffalo Grove, Ill.) under long daylight conditions with either normal temperature conditions (Control) or high temperature conditions (High temperature).

FIGS. 8B-8D are graphs showing the (B) plant heights, (C) above-ground vegetative biomasses and (D) root biomasses (mean±standard deviation, n=6) of wild-type rice plants (WT) and transgenic rice plants overexpressing OsMYB55 (OsMYB55-4; OsMYB55-11) following germination under normal growth conditions and four weeks of growth in Turface® MVP® (PROFILE Products, LLC, Buffalo Grove, Ill.) under long daylight conditions with either normal temperature conditions (Control) or high temperature conditions (High temperature).

FIG. 9A shows wild-type rice plants (WT) and transgenic rice plants overexpressing OsMYB55 (MYB55-4; MYB55-11) following four weeks of growth in peat-moss:vermiculite (1:4) under normal daylight conditions with either normal temperature conditions (“29”) or high temperature conditions (“35”).

FIG. 9B shows wild-type rice plants (WT) and transgenic rice plants overexpressing OsMYB55 (MYB55-4; MYB55-11) following four weeks of growth in peat-moss:vermiculite (1:4) under normal daylight conditions with high temperature conditions.

FIG. 9C is a graph showing the above-ground vegetative growth dry biomass (mean±standard deviation, n=6) of wild-type rice plants (WT) and transgenic rice plants overexpressing OsMYB55 (MYB55-4; MYB55-11) following four weeks of growth under normal daylight conditions with either normal temperature conditions (Control) or high temperature conditions (High temperature).

FIG. 9D is a graph showing plant height and leaf sheath length (mean±standard deviation, n=6) of wild-type rice plants (WT) and transgenic rice plants overexpressing OsMYB55 (MYB55-4; MYB55-11) following four weeks of growth under normal daylight conditions with high temperature conditions.

FIG. 10A shows the rice panicles of a wild-type rice plant (leftmost plant in each grouping) and transgenic rice plants overexpressing OsMYB55 (two rightmost plants in each grouping) following nine weeks of growth under normal daylight conditions with either normal temperature conditions (left) or high temperature conditions (right).

FIG. 10B shows the rice panicles of a wild-type rice plant following 11 weeks of growth under normal growth conditions.

FIG. 10C shows the rice panicles of a wild-type rice plant following 11 weeks of growth under long daylight conditions with high temperature conditions.

FIG. 10D shows the rice panicles of a wild-type rice plant following 11 weeks of growth under normal daylight conditions with high temperature conditions.

FIG. 10E shows the rice panicles of a wild-type rice plant following 17 weeks of growth under normal growth conditions.

FIG. 10F shows the rice panicles of a wild-type rice plant grown for 17 weeks under long daylight conditions with high temperature conditions.

FIG. 10G shows the rice panicles of a wild-type rice plant grown for 17 weeks under normal daylight conditions with high temperature conditions.

FIGS. 11A-11B are graphs showing the percent reduction in (A) total dry biomasses and (B) grain yields of wild-type rice plants (WT) and transgenic rice plants overexpressing OsMYB55 (OsMYB55-4; OsMYB55-11) grown under normal daylight conditions with high temperature conditions for four weeks and then grown under normal growth conditions until harvest (approximately 12 additional weeks) as compared to equivalent plants grown under normal growth conditions until harvest (approximately 16 weeks).

FIG. 12 is a graph showing the relative transcript levels (mean±standard deviation) of OsMYB55 in the leaves of wild-type rice plants (WT) and transgenic rice plants expressing OsMYB55 interference RNA (OsMYB55-RNAi) (OsMYB55::RNAi-12; OsMYB55::RNAi-16) grown under normal growth conditions. The OsMYB55 transcript level of OsMYB55::RNAi-12 was used as a reference value to calculate the relative transcripts levels.

FIG. 13A is a graph showing the total amino acid content (mean±standard deviation, n=3) of leaves from wild-type rice plants (WT) and transgenic rice plants overexpressing OsMYB55 (OsMYB55-4; OsMYB55-11) grown for four weeks under long daylight conditions with either normal temperature conditions (Control) or high temperature conditions (High temperature).

FIGS. 13B-13D are graphs showing the relative expression levels (mean±standard deviation, n=3) of (B) Oryza sativa glutamine synthetase (OsGs1;2), (C) Oryza sativa class I glutamine amidotransferase (OsGAT1) and (0) Oryza sativa glutamine decarboxylase 3 (OsGAD3) in the leaves of wild-type rice plants (WT) and transgenic rice plants overexpressing OsMYB55 (OsMYB55-4; OsMYB55-11) grown under long daylight conditions with normal temperature conditions for four weeks (Control) or grown under long daylight conditions with normal temperature conditions for four weeks and then exposed to 45° C. for 1, 6 or 24 hours. The results depicted in the graph are representative of similar results from three independent experiments.

FIG. 14A shows electrophoretic mobility shift assays using varying amounts of recombinant OsMYB55 (0-40 μg) and 200 ng of DNA containing one copy of a promoter region isolated from (I) OsGs1;2, (II) OsGAT1 or (Ill) OsGAD3.

FIG. 14B is a graph showing the expression levels (mean±standard deviation, n=6) of GUS in a transient gene expression assay wherein four-week-old tobacco plants were co-transformed with a vector comprising OsMYB55 and a GUS-reporting vector comprising GUS under the control of a promoter region isolated from OsGs1;2, OsGAT1 or OsGAD3.

FIG. 15A is a graph showing the glutamic acid content (mean±standard deviation) of leaves from wild-type rice plants (WT) and transgenic rice plants overexpressing OsMYB55 (OsMYB55-4; OsMYB55-11) following four weeks of growth under long daylight conditions with either normal temperature conditions (Control) or high temperature conditions (High temperature).

FIG. 15B is a graph showing the γ-aminobutyric acid (GABA) content (mean±standard deviation) of leaves from wild-type rice plants (WT) and transgenic rice plants overexpressing OsMYB55 (OsMYB55-4; OsMYB55-11) following four weeks of growth under long daylight conditions with either normal temperature conditions (Control) or high temperature conditions (High temperature).

FIG. 15C is a graph showing the arginine content (mean±standard deviation) of leaves from wild-type rice plants (WT) and transgenic rice plants overexpressing OsMYB55 (OsMYB55-4; OsMYB55-11) following four weeks of growth under long daylight conditions with either normal temperature conditions (Control) or high temperature conditions (High temperature).

FIG. 15D is a graph showing the proline content (mean±standard deviation) of leaves from wild-type rice plants (WT) and transgenic rice plants overexpressing OsMYB55 (OsMYB55-4; OsMYB55-11) following four weeks of growth under long daylight conditions with either normal temperature conditions (Control) or high temperature conditions (High temperature).

FIGS. 16A-16B are Venn diagrams representing the number of genes that were significantly (A) up-regulated or (B) down-regulated in wild-type rice plants (VVT) and transgenic rice plants overexpressing OsMYB55 (OsMYB55) following four weeks of growth under normal growth conditions and then exposure to 45° C. for one hour.

FIGS. 17A-H shows the amino acid and coding sequences for various plant MYB55 homologues. FIG. 17A depicts the amino acid (SEQ ID NO:6) and cDNA (SEQ ID NO:14) sequences for a MYB55 homologue from Sorghum bicolor. FIG. 17B depicts the amino acid (SEQ ID NO:7) and cDNA (SEQ ID NO:15) sequences for a MYB55 homologue from Zea mays. FIG. 17C depicts the amino acid (SEQ ID NO:8) sequence for a MYB55 homologue from Vitis vinifera. FIG. 17D depicts the amino acid (SEQ ID NO:9) and cDNA (SEQ ID NO:16) sequences for a MYB55 homologue (previously designated MYB133) from Populus trichocarps. FIG. 17E depicts the amino acid (SEQ ID NO:10) and cDNA (SEQ ID NO:17) sequences for a MYB55 homologue (previously designated MYB24) from Malus x domestica. FIG. 17F depicts the amino acid (SEQ ID NO:11) and cDNA (SEQ ID NO:18) sequences for a MYB55 homologue (previously designated DcMYB4) from Glycine max. FIG. 17G depicts the amino acid (SEQ ID NO:12) and cDNA (SEQ ID NQ:19) sequences for a MYB55 homologue from Daucus carota. FIG. 17H depicts the amino acid (SEQ ID NO:13) and cDNA (SEQ ID NO:20) sequences for a MYB55 homologue (previously designated MYB36) from Arabidopsis thaliana.

### DETAILED DESCRIPTION OF THE INVENTION

It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

### I. DEFINITIONS

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage or time period and the like, is meant to encompass variations of ±20%, ±10%, ±5%, ±1%, ±0.5%, or even ±0.1% of the specified amount.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim or the description of this invention is not intended to be interpreted to be equivalent to “comprising.”

Unless indicated otherwise, the terms “heat stress” and “high temperature” (and similar terms) refer to exposing a plant, plant part or plant cell to elevated temperatures that are higher than is optimal for the plant species and/or variety and/or developmental stage. In representative embodiments, the plant, plant part or plant cell is exposed to a high temperature for an insufficient time to result in heat stress (e.g., reduced yield). In embodiments of the invention, the plant, plant part or plant cell is exposed to a high temperature for a sufficient time to result in heat stress.

For example, in embodiments of the invention, a plant can be exposed to high temperature for a sufficient period of time to produce heat stress in the plant and result in an adverse effect on plant function, development and/or performance, e.g., reduced cell division, size (e.g., reduced plant height) and/or number of plants and/or parts thereof and/or an impairment in an agronomic trait such as reduced yield, fruit drop, fruit size and/or number, seed size and/or number, quality of produce due to appearance and/or texture and/or increased flower abortion. Plants, plant parts and plant cells may be exposed or subjected to heat stress or high temperature under a variety of circumstances, e.g., a cultivated plant exposed to heat stress or high temperature due to ambient temperatures; a plant, plant part or plant cell exposed to heat stress or high temperature during harvesting, processing, storage and/or shipping; or a plant, plant part or plant cell exposed to heat stress or high temperature to achieve a desired effect (e.g., inducing the activity of a heat-inducible promoter).

Those skilled in the art will recognize that the terms “heat stress” and “high temperature” are not absolute and may vary with the plant species, plant variety, developmental stage, water availability, soil type, geographic location, day length, season, the presence of other abiotic and/or biotic stressors, and other parameters that are well within the level of skill in the art. Thus, while one species may be severely impacted by a temperature of 23° C., another species may not be impacted until at least 30° C., and the like. Typically, temperatures above 30° C. result in a significant reduction in the yields of most important crops.

In embodiments of the invention, exposure to heat stress or high temperature comprises exposing a plant, plant part or plant cell to temperatures of at least about 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C. or 55° C. In embodiments of the invention, exposure to heat stress or high temperature refers to temperatures from about 30° C. to about 45° C., 46° C., 47° C., 48° C., 49° C., 50° C., 51° C., 52° C., 53° C., 54° C. or 55° C.; from about 31° C. to about 45° C., 46° C., 47° C., 48° C., 49° C., 50° C., 51° C., 52° C., 53° C., 54° C. or 55° C.; from about 32° C. to about 45° C., 46° C., 47° C., 48° C., 49° C., 50° C., 51° C., 52° C., 53° C., 54° C. or 55° C.; from about 33° C. to about 45° C., 46° C., 47° C., 48° C., 49° C., 50° C., 51° C., 52° C., 53° C., 54° C. or 55° C.; from about 34° C. to about 45° C., 46° C., 47° C., 48° C., 49° C., 50° C., 51° C., 52° C., 53° C., 54° C. or 55° C.; from about 35° C. to about 45° C., 46° C., 47° C., 48° C., 49° C., 50° C., 51° C., 52° C., 53° C., 54° C. or 55° C.; from about 36° C. to about 45° C., 46° C., 47° C., 48° C., 49° C., 50° C., 51° C., 52° C., 53° C., 54° C. or 55° C.; from about 37° C. to about 45° C., 46° C., 47° C., 48° C., 49° C., 50° C., 51° C., 52° C., 53° C., 54° C. or 55° C.; from about 38° C. to about 45° C., 46° C., 47° C., 48° C., 49° C., 50° C., 51° C., 52° C., 53° C., 54° C. or 55° C.; from about 39° C. to about 45° C., 46° C., 47° C., 48° C., 49° C., 50° C., 51° C., 52° C., 53° C., 54° C. or 55° C.; or from about 40° C. to about 45° C., 46° C., 47° C., 48° C., 49° C., 50° C., 51° C., 52° C., 53° C., 54° C. or 55° C. In representative embodiments, the temperatures above refer to day-time temperatures.

In additional embodiments, exposure to heat stress or high temperature comprises exposing a plant, plant part or plant cell to night-time temperatures of about 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C. or 55° C. In embodiments of the invention, heat stress or high temperature refers to night-time temperatures from about 25° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 26° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 27° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 28° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 29° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 30° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 31° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 32° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 33° C. to about 40° C., 41° C., 42° C., 43° C., 44° C., 45° C., 46° C., 47° C., 48° C., 49° C., 50° C., 51° C., 52° C., 53° C., 54° C. or 55° C.; from about 34° C. to about 40° C., 41° C., 42° C., 43° C., 44° C., 45° C., 46° C., 47° C., 48° C., 49° C., 50° C., 51° C., 52° C., 53° C., 54° C. or 55° C.; or from about 35° C. to about 40° C., 41° C., 42° C., 43° C., 44° C., 45° C., 46° C., 47° C., 48° C., 49° C., 50° C., 51° C., 52° C., 53° C., 54° C. or 55° C.

The plant, plant part or plant cell can be exposed to the heat stress or high temperature for any period of time. In exemplary embodiments, the plant, plant part or plant cell is exposed to heat stress or high temperature for a period of at least about 1, 2, 5, 10, 15, 20, 30, 40, 50, 60, 90 or 120 minutes or longer; at least about 1, 2, 5, 10, 15, 18, 24, 48, 72 or 96 hours or longer; at least about 1, 2, 3, 4, 7, 10, 14, 21 or 30 days or longer, at least about 1, 2, 3, 4, 5 or 6 weeks or longer; or at least about 1, 2, 3 or 4 months or longer.

Optionally, the plant, plant part or plant cell is exposed to heat stress or high temperature during the vegetative stage of growth. By exposing a plant, plant part or plant cell to heat stress or high temperature during the vegetative stage of growth it is meant that the plant, plant part or plant cell is subjected to the heat stress or high temperature for all or a portion of the vegetative stage of growth, e.g., for a period of at least about 1, 2, 5, 10, 15, 20, 30, 40, 50, 60, 90 or 120 minutes or longer; at least about 1, 2, 5, 10, 15, 18, 24, 48, 72 or 96 hours or longer; at least about 1, 2, 3, 4, 7, 10, 14, 21 or 30 days or longer, at least about 1, 2, 3, 4, 5 or 6 weeks or longer; or at least about 1, 2, 3 or 4 months or longer.

In representative embodiments, the plant, plant part or plant cell is not exposed to heat stress or high temperature during inflorescence and/or seed set stages.

The present invention encompasses heat stress or high temperature conditions produced by any combination of the temperatures and time periods described herein.

In representative embodiments, the plant, plant part or plant cell is exposed to heat stress or high temperature comprising a day-time temperature of about 35° C. and a night-time temperature of about 26° C., e.g., for a period of about one, two, three, four weeks, or longer. In embodiments of the invention, the plant, plant part or plant cell is subject to heat stress or high temperature comprising exposure to about 45° C. for a period of at least about 5, 10, 15, 20, 30, 40, 50, 60, 90 or 120 minutes or longer.

Those skilled in the art will appreciate that generally the plant, plant part or plant cell is exposed to a sub-lethal level of heat stress or high temperature (e.g., that is not lethal to the plant, plant part or plant cell).

The term “increased tolerance to heat stress,” “increasing tolerance to heat stress,” “increased tolerance to high temperature,” or “increasing tolerance to high temperature” (and similar terms) as used herein refers to the ability of a plant, plant part or plant cell exposed to heat stress or high temperature and comprising a nucleic acid (e.g., isolated nucleic acid), expression cassette or vector as described herein to withstand a given heat stress or high temperature better than a control plant, plant part or plant cell (i.e., a plant, plant part or plant cell that does not comprise a nucleic acid, expression cassette or vector as described herein). Increased tolerance to heat stress or high temperature can be measured using a variety of parameters including, but not limited to, increased cell division, size (e.g., plant height) and/or number of plants and/or parts thereof and/or an improvement in an agronomic trait such as increased yield, fruit drop, fruit size and/or number, seed size and/or number and/or increased quality of produce due to appearance and/or texture and/or reduced flower abortion. In embodiments of the invention, increased tolerance to heat stress or high temperature can be assessed in terms of an increase in plant height, plant biomass (e.g., dry biomass) and/or grain yield. Increases in these indices (e.g., yield, plant size, plant height, plant biomass, grain yield, and the like) may indicate that there is an increase as compared with a control plant, plant part or plant cell that has not been subject to heat stress or high temperature and/or may indicate that there is an increase as compared with a control plant, plant part or plant cell that has been subject to heat stress or high temperature but does not comprise a nucleic acid, expression cassette or vector as described herein. In other words, there may be a reduction as compared with a plant, plant part or plant cell that has not been exposed to heat stress or high temperature but the decrease is less than in a plant, plant part or plant cell subject to the heat stress or high temperature that does not comprise a nucleic acid, expression cassette or vector as described herein.

“Yield” as used herein refers to the production of a commercially and/or agriculturally important plant, plant biomass (e.g., dry biomass), plant part (e.g., roots, tubers, seed, leaves, fruit, flowers), plant material (e.g., an extract) and/or other product produced by the plant (e.g., a recombinant polypeptide). In embodiments of the invention, “increased yield” is assessed in terms of an increase in plant height.

An “increase in amino acid content,” “increased amino acid content” and similar terms as used herein refers to an elevation in the amount and/or concentration of amino acids. The increase can be an increase in total amino acid content and/or can be an increase in the content of one or more individual amino acids found in plants including without limitation glutamic acid, arginine, gamma-amino butyric acid (GABA), proline, aspartic acid, asparagine, threonine, leucine, isoleucine, threonine, methionine, alanine, valine, glycine, lysine, serine, cysteine, histidine, tryptophan, tyrosine, phenylalanine, ornithine, citrulline, or any combination thereof. In embodiments of the invention, there is an increase in glutamic acid, arginine, GABA and/or proline content. The increase in amino acid content can be in the total plant biomass and/or in one or more parts or tissues thereof (e.g., leaves, leaf sheaths and/or roots). The increase in amino acid content can be assessed with respect to any suitable control, e.g., a plant, plant part or plant cell that does not comprise a nucleic acid, expression cassette or vector as described herein. In embodiments of the invention, the plant has been exposed to heat stress or high temperature. In embodiments of the invention, the plant has not been exposed to heat stress or high temperature.

The term “modulate” (and grammatical variations) refers to an increase or decrease.

As used herein, the terms “increase,” “increases,” “increased,” “increasing” and similar terms indicate an elevation of at least about 25%, 50%, 75%, 100%, 150%, 200%, 300%, 400%, 500% or more.

As used herein, the terms “reduce,” “reduces,” “reduced,” “reduction” and similar terms mean a decrease of at least about 25%, 35%, 50%, 75%, 80%, 85%, 90%, 95%, 97% or more. In particular embodiments, the reduction results in no or essentially no (i.e., an insignificant amount, e.g., less than about 10% or even 5%) detectable activity or amount.

As used herein, the term “heterologous” means foreign, exogenous, non-native and/or non-naturally occurring.

As used here, “homologous” means native. For example, a homologous nucleotide sequence or amino acid sequence is a nucleotide sequence or amino acid sequence naturally associated with a host cell into which it is introduced, a homologous promoter sequence is the promoter sequence that is naturally associated with a coding sequence, and the like.

As used herein a “chimeric nucleic acid,” “chimeric nucleotide sequence” or “chimeric polynucleotide” comprises a promoter operably linked to a nucleotide sequence of interest that is heterologous to the promoter (or vice versa). In particular embodiments, the “chimeric nucleic acid,” “chimeric nucleotide sequence” or “chimeric polynucleotide” comprises a nucleic acid as described herein operably associated with a heterologous promoter sequence.

A “promoter” is a nucleotide sequence that controls or regulates the transcription of a nucleotide sequence (i.e., a coding sequence) that is operatively associated with the promoter. The coding sequence may encode a polypeptide and/or a functional RNA. Typically, a “promoter” refers to a nucleotide sequence that contains a binding site for RNA polymerase II and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

“Nucleotide sequence of interest” refers to any nucleotide sequence which, when introduced into a plant, confers upon the plant a desired characteristic, for example, increased tolerance to heat stress, high temperature and/or drought. The “nucleotide sequence of interest” can encode a polypeptide and/or an inhibitory polynucleotide (e.g., a functional RNA).

A “heterologous nucleotide sequence of interest” is heterologous (e.g., foreign) to the promoter with which it is operatively associated.

A “functional” RNA includes any untranslated RNA that has a biological function in a cell, e.g., regulation of gene expression. Such functional RNAs include but are not limited to RNAi (e.g., siRNA, shRNA), miRNA, antisense RNA, ribozymes, RNA aptamers and the like.

By “operably linked” or “operably associated” as used herein, it is meant that the indicated elements are functionally related to each other, and are also generally physically related. For example, a promoter is operatively linked or operably associated to a coding sequence (e.g., nucleotide sequence of interest) if it controls the transcription of the sequence. Thus, the term “operatively linked” or “operably associated” as used herein, refers to nucleotide sequences on a single nucleic acid molecule that are functionally associated. Those skilled in the art will appreciate that the control sequences (e.g., promoter) need not be contiguous with the coding sequence, as long as they functions to direct the expression thereof. Thus, for example, intervening untranslated, yet transcribed, sequences can be present between a promoter and a coding sequence, and the promoter sequence can still be considered “operably linked” to the coding sequence.

By the term “express,” “expressing” or “expression” (or other grammatical variants) of a nucleic acid coding sequence, it is meant that the sequence is transcribed. In particular embodiments, the terms “express,” “expressing” or “expression” (or other grammatical variants) can refer to both transcription and translation to produce an encoded polypeptide.

“Wild-type” nucleotide sequence or amino acid sequence refers to a naturally occurring (“native”) or endogenous nucleotide sequence (including a cDNA corresponding thereto) or amino acid sequence.

The terms “nucleic acid,” “polynucleotide” and “nucleotide sequence” are used interchangeably herein unless the context indicates otherwise. These terms encompass both RNA and DNA, including cDNA, genomic DNA, partially or completely synthetic (e.g., chemically synthesized) RNA and DNA, and chimeras of RNA and DNA. The nucleic acid, polynucleotide or nucleotide sequence may be double-stranded or single-stranded, and further may be synthesized using nucleotide analogs or derivatives (e.g., inosine or phosphorothioate nucleotides). Such nucleotides can be used, for example, to prepare nucleic acids, polynucleotides and nucleotide sequences that have altered base-pairing abilities or increased resistance to nucleases. The present invention further provides a nucleic acid, polynucleotide or nucleotide sequence that is the complement (which can be either a full complement or a partial complement) of a nucleic acid, polynucleotide or nucleotide sequence of the invention. Nucleotide sequences are presented herein by single strand only, in the 5′ to 3′ direction, from left to right, unless specifically indicated otherwise. Nucleotides and amino acids are represented herein in the manner recommended by the IUPAC-IUB Biochemical Nomenclature Commission, or (for amino acids) by either the one-letter code, or the three letter code, both in accordance with 37 CFR §1.822 and established usage.

The nucleic acids and polynucleotides of the invention are optionally isolated. An “isolated” nucleic acid molecule or polynucleotide is a nucleic acid molecule or polynucleotide that, by the hand of man, exists apart from its native environment and is therefore not a product of nature. An isolated nucleic acid molecule or isolated polynucleotide may exist in a purified form or may exist in a non-native environment such as, for example, a recombinant host cell. Thus, for example, the term “isolated” means that it is separated from the chromosome and/or cell in which it naturally occurs. A nucleic acid or polynucleotide is also isolated if it is separated from the chromosome and/or cell in which it naturally occurs and is then inserted into a genetic context, a chromosome, a chromosome location, and/or a cell in which it does not naturally occur. The recombinant nucleic acid molecules and polynucleotides of the invention can be considered to be “isolated.”

Further, an “isolated” nucleic acid or polynucleotide can be a nucleotide sequence (e.g., DNA or RNA) that is not immediately contiguous with nucleotide sequences with which it is immediately contiguous (one on the 5′ end and one on the 3′ end) in the naturally occurring genome of the organism from which it is derived. The “isolated” nucleic acid or polynucleotide can exist in a cell (e.g., a plant cell), optionally stably incorporated into the genome. According to this embodiment, the “isolated” nucleic acid or polynucleotide can be foreign to the cell/organism into which it is introduced, or it can be native to an the cell/organism, but exist in a recombinant form (e.g., as a chimeric nucleic acid or polynucleotide) and/or can be an additional copy of an endogenous nucleic acid or polynucleotide. Thus, an “isolated nucleic acid molecule” or “isolated polynucleotide” can also include a nucleotide sequence derived from and inserted into the same natural, original cell type, but which is present in a non-natural state, e.g., present in a different copy number, in a different genetic context and/or under the control of different regulatory sequences than that found in the native state of the nucleic acid molecule or polynucleotide.

In representative embodiments, the “isolated” nucleic acid or polynucleotide is substantially free of cellular material (including naturally associated proteins such as histones, transcription factors, and the like), viral material, and/or culture medium (when produced by recombinant DNA techniques), or chemical precursors or other chemicals (when chemically synthesized). Optionally, in representative embodiments, the isolated nucleic acid or polynucleotide is at least about 10%, 20%, 30%, 40%, 50%, 60%, 70%, 80%, 90%, 95% or more pure.

As used herein, the term “recombinant” nucleic acid, polynucleotide or nucleotide sequence refers to a nucleic acid, polynucleotide or nucleotide sequence that has been constructed, altered, rearranged and/or modified by genetic engineering techniques. The term “recombinant” does not refer to alterations that result from naturally occurring events, such as spontaneous mutations, or from non-spontaneous mutagenesis.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in the cell, i.e., capable of nucleic acid replication under its own control. The term “vector” includes both viral and nonviral (e.g., plasmid) nucleic acid molecules for introducing a nucleic acid into a cell in vitro, ex vivo, and/or in vivo, and is optionally an expression vector. A large number of vectors known in the art may be used to manipulate, deliver and express polynucleotides. Vectors may be engineered to contain sequences encoding selectable markers that provide for the selection of cells that contain the vector and/or have integrated some or all of the nucleic acid of the vector into the cellular genome. Such markers allow identification and/or selection of host cells that incorporate and express the proteins encoded by the marker. A “recombinant” vector refers to a viral or non-viral vector that comprises one or more nucleotide sequences of interest (e.g., transgenes), e.g., two, three, four, five or more nucleotide sequences of interest.

Viral vectors have been used in a wide variety of gene delivery applications in cells, as well as living animal subjects. Plant viral vectors that can be used include, but are not limited to, Agrobacterium tumefaciens, Agrobacterium rhizogenes and geminivirus vectors. Non-viral vectors include, but are not limited to, plasmids, liposomes, electrically charged lipids (cytofectins), nucleic acid-protein complexes, and biopolymers. In addition to a nucleic acid of interest, a vector may also comprise one or more regulatory regions, and/or selectable markers useful in selecting, measuring, and monitoring nucleic acid transfer results (e.g., delivery to specific tissues, duration of expression, etc.).

The term “fragment,” as applied to a nucleic acid or polynucleotide, will be understood to mean a nucleotide sequence of reduced length relative to the reference or full-length nucleotide sequence and comprising, consisting essentially of and/or consisting of contiguous nucleotides from the reference or full-length nucleotide sequence. Such a fragment according to the invention may be, where appropriate, included in a larger polynucleotide of which it is a constituent. In some embodiments, such fragments can comprise, consist essentially of, and/or consist of oligonucleotides having a length that is greater than and/or is at least about $, 10, 12, 15, 16, 17, 18, 19, 20, 25, 30, 35, 40, 45, 50, 75, 100, 125, 150, 175, 200, 250, 300, 350, 400, 450, 500, 600, 700, 800, 900, 1000, 1100, 1200, 1300, 1400 or 1500 nucleotides (optionally, contiguous nucleotides) or more from the reference or full-length nucleotide sequence, as long as the fragment is shorter than the reference or full-length nucleotide sequence. In representative embodiments, the fragment is a biologically active nucleotide sequence, as that term is described herein.

A “biologically active” nucleotide sequence is one that substantially retains at least one biological activity normally associated with the wild-type nucleotide sequence, for example, the nucleotide sequence encodes a polypeptide that has enzyme activity, binding activity (e.g., DNA binding activity), transcription factor activity (e.g., ability to increase transcription), ability to increase tolerance to heat stress, and/or ability to increase amino acid content. In particular embodiments, the “biologically active” nucleotide sequence substantially retains all of the biological activities possessed by the unmodified sequence. By “substantially retains” biological activity, it is meant that the nucleotide sequence retains at least about 50%, 60%, 75%, 85%, 90%, 95%, 97%, 98%, 99%, or more, of the biological activity of the native nucleotide sequence (and can even have a higher level of activity than the native nucleotide sequence).

Two nucleotide sequences are said to be “substantially identical” to each other when they share at least about 60%, 70%, 75%, 80%, 85%, 90%, 95%, 97%, 98%, 99% or even 100% sequence identity. In embodiments of the invention, a “substantially identical” nucleotide sequence has about 1, 2, 3, 4, 5, 6, 7, 8, 9 or 10 nucleotide substitutions, insertions and/or deletions, taken individually or collectively, as compared with a reference sequence.

Two amino acid sequences are said to be “substantially identical” or “substantially similar” to each other when they share at least about 60%, 70%, 75%, 80%, 85%, 90%, 95%, 97%, 98%, 99% or even 100% sequence identity or similarity, respectively. In embodiments of the invention, a “substantially identical” amino acid sequence has about 1, 2, 3, 4, 5, 6, 7, 8, 9 or 10 amino acid substitutions, insertions and/or deletions, taken individually or collectively, as compared with a reference sequence. In embodiments of the invention, a “substantially similar” amino acid sequence has about 1, 2, 3, 4, 5, 6, 7, 8, 9 or 10 amino acid substitutions, insertions and/or deletions, taken individually or collectively, as compared with a reference sequence, where the amino acid substitutions can be conservative and/or non-conservative substitutions.

As used herein “sequence identity” refers to the extent to which two optimally aligned polynucleotide or polypeptide sequences are invariant throughout a window of alignment of components, e.g., nucleotides or amino acids.

As used herein “sequence similarity” is similar to sequence identity (as described herein), but permits the substitution of conserved amino acids (e.g., amino acids whose side chains have similar structural and/or biochemical properties), which are well-known in the art.

As is known in the art, a number of different programs can be used to identify whether a nucleic acid has sequence identity or an amino acid sequence has sequence identity or similarity to a known sequence. Sequence identity or similarity may be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al. Nucleic Acids Res. 25, 3389-3402 (1997).

The CLUSTAL program can also be used to determine sequence similarity. This algorithm is described by Higgins et al. (1988) Gene 73:237; Higgins et al. (1989) CABIOS 5:151-153; Corpet et al. (1988) Nucleic Acids Res. 16: 10881-90; Huang et al. (1992) CABIOS 8: 155-65; and Pearson et al. (1994) Meth. Mol. Biol. 24: 307-331.

The alignment may include the introduction of gaps in the sequences to be aligned. In addition, for sequences which contain either more or fewer nucleotides than the nucleic acids disclosed herein, it is understood that in one embodiment, the percentage of sequence identity will be determined based on the number of identical nucleotides acids in relation to the total number of nucleotide bases. Thus, for example, sequence identity of sequences shorter than a sequence specifically disclosed herein, will be determined using the number of nucleotide bases in the shorter sequence, in one embodiment. In percent identity calculations relative weight is not assigned to various manifestations of sequence variation, such as, insertions, deletions, substitutions, etc.

Two nucleotide sequences can also be considered to be substantially identical when the two sequences hybridize to each other under stringent conditions. A nonlimiting example of “stringent” hybridization conditions include conditions represented by a wash stringency of 50% Formamide with 5×Denhardt's solution, 0.5% SDS and 1×SSPE at 42° C. “Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). In some representative embodiments, two nucleotide sequences considered to be substantially identical hybridize to each other under highly stringent conditions. Generally, highly stringent hybridization and wash conditions are selected to be about 5° C. lower than the thermal melting point (Tₘ) for the specific sequence at a defined ionic strength and pH.

As used herein, the term “polypeptide” encompasses both peptides and proteins (including fusion proteins), unless indicated otherwise.

A “fusion protein” is a polypeptide produced when two heterologous nucleotide sequences or fragments thereof coding for two (or more) different polypeptides not found fused together in nature are fused together in the correct translational reading frame.

The polypeptides of the invention are optionally “isolated.” An “isolated” polypeptide is a polypeptide that, by the hand of man, exists apart from its native environment and is therefore not a product of nature. An isolated polypeptide may exist in a purified form or may exist in a non-native environment such as, for example, a recombinant host cell. The recombinant polypeptides of the invention can be considered to be “isolated.”

In representative embodiments, an “isolated” polypeptide means a polypeptide that is separated or substantially free from at least some of the other components of the naturally occurring organism or virus, for example, the cell or viral structural components or other polypeptides or nucleic acids commonly found associated with the polypeptide. In particular embodiments, the “isolated” polypeptide is at least about 1%, 5%, 10%, 25%, 50%, 60%, 70%, 75%, 80%, 85%, 90%, 95%, 97%, 98%, 99% or more pure (w/w). In other embodiments, an “isolated” polypeptide indicates that at least about a 5-fold, 10-fold, 25-fold, 100-fold, 1000-fold, 10,000-fold, or more enrichment of the protein (w/w) is achieved as compared with the starting material. In representative embodiments, the isolated polypeptide is a recombinant polypeptide produced using recombinant nucleic acid techniques. In embodiments of the invention, the polypeptide is a fusion protein.

The term “fragment,” as applied to a polypeptide, will be understood to mean an amino acid of reduced length relative to a reference polypeptide or the full-length polypeptide and comprising, consisting essentially of, and/or consisting of a sequence of contiguous amino acids from the reference or full-length polypeptide. Such a fragment according to the invention may be, where appropriate, included as part of a fusion protein of which it is a constituent. In some embodiments, such fragments can comprise, consist essentially of, and/or consist of polypeptides having a length of at least about 50, 60, 70, 80, 90, 100, 110, 120, 130, 140, 150, 160, 170, 180, 190, 200, 225, 250, 275, 300, 325, 375, 400, 425, 450, 475, or 500 amino acids (optionally, contiguous amino acids) from the reference or full-length polypeptide, as long as the fragment is shorter than the reference or full-length polypeptide. In representative embodiments, the fragment is biologically active, as that term is defined herein.

A “biologically active” polypeptide is one that substantially retains at least one biological activity normally associated with the wild-type polypeptide, for example, enzyme activity, binding activity (e.g., DNA binding activity), transcription factor activity (e.g., ability to increase transcription), ability to increase tolerance to heat stress, and/or ability to increase amino acid content. In particular embodiments, the “biologically active” polypeptide substantially retains all of the biological activities possessed by the unmodified (e.g., native) sequence. By “substantially retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide).

As used herein, an “equivalent” amino acid sequence refers to an amino acid sequence that is altered by one or more amino acids. The equivalent may optionally have “conservative” changes, wherein a substituted amino acid has similar structural or chemical properties. In particular, such changes can be guided by known similarities between amino acids in physical features such as charge density, hydrophobicity/hydrophilicity, size and configuration, so that amino acids are substituted with other amino acids having essentially the same functional properties. For example: Ala may be replaced with Val or Ser; Val may be replaced with Ala, Leu, Met, or Ile, preferably Ala or Leu; Leu may be replaced with Ala, Val or Ile, preferably Val or Ile; Gly may be replaced with Pro or Cys, preferably Pro; Pro may be replaced with Gly, Cys, Ser, or Met, preferably Gly, Cys, or Ser; Cys may be replaced with Gly, Pro, Ser, or Met, preferably Pro or Met; Met may be replaced with Pro or Cys, preferably Cys; His may be replaced with Phe or Gln, preferably Phe; Phe may be replaced with His, Tyr, or Trp, preferably His or Tyr; Tyr may be replaced with His, Phe or Trp, preferably Phe or Trp; Trp may be replaced with Phe or Tyr, preferably Tyr; Asn may be replaced with Gln or Ser, preferably Gln; Gln may be replaced with His, Lys, Glu, Asn, or Ser, preferably Asn or Ser; Ser may be replaced with Gln, Thr, Pro, Cys or Ala; Thr may be replaced with Gln or Ser, preferably Ser; Lys may be replaced with Gln or Arg; Arg may be replaced with Lys, Asp or Glu, preferably Lys or Asp; Asp may be replaced with Lys, Arg, or Glu, preferably Arg or Glu; and Glu may be replaced with Arg or Asp, preferably Asp. Once made, changes can be routinely screened to determine their effects on function.

Alternatively, an equivalent amino acid sequence may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE™ software.

In making amino acid substitutions, the hydropathic index of amino acids can be considered. The importance of the hydropathic amino acid index in conferring interactive biologic function on a protein is generally understood in the art (see, Kyte and Doolittle, (1982) J. Mol. Biol. 157:105). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, Id.), and these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is also understood in the art that the substitution of amino acids can be made on the basis of hydrophilicity. U.S. Pat. No. 4,554,101 states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±I); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

“Introducing” in the context of a plant cell, plant tissue, plant part and/or plant means contacting a nucleic acid molecule with the plant cell, plant tissue, plant part, and/or plant in such a manner that the nucleic acid molecule gains access to the interior of the plant cell or a cell of the plant tissue, plant part or plant. Where more than one nucleic acid molecule is to be introduced, these nucleic acid molecules can be assembled as part of a single polynucleotide or nucleic acid construct, or as separate polynucleotide or nucleic acid constructs, and can be located on the same or different nucleic acid constructs. To illustrate, these polynucleotides can be introduced into plant cells in a single transformation event, in separate transformation events, or as part of a breeding protocol.

The term “transformation” as used herein refers to the introduction of a heterologous and/or isolated nucleic acid into a cell. Transformation of a cell may be stable or transient. Thus, a transgenic plant cell, plant tissue, plant part and/or plant of the invention can be stably transformed or transiently transformed.

“Transient transformation” in the context of a polynucleotide means that a polynucleotide is introduced into the cell and does not integrate into the genome of the cell.

As used herein, “stably introducing,” “stably introduced,” “stable transformation” or “stably transformed” (and similar terms) in the context of a polynucleotide introduced into a cell, means that the introduced polynucleotide is stably integrated into the genome of the cell (e.g., into a chromosome or as a stable-extra-chromosomal element). As such, the integrated polynucleotide is capable of being inherited by progeny cells and plants.

“Genome” as used herein includes the nuclear and/or plastid genome, and therefore includes integration of a polynucleotide into, for example, the chloroplast genome. Stable transformation as used herein can also refer to a polynucleotide that is maintained extrachromosomally, for example, as a minichromosome.

As used herein, the terms “transformed” and “transgenic” refer to any plant, plant cell, plant tissue (including callus), or plant part that contains all or part of at least one recombinant or isolated nucleic acid, polynucleotide or nucleotide sequence. In representative embodiments, the recombinant or isolated nucleic acid, polynucleotide or nucleotide sequence is stably integrated into the genome of the plant (e.g., into a chromosome or as a stable extra-chromosomal element), so that it is passed on to subsequent generations of the cell or plant.

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs plant cell tissue cultures, plant calli, plant clumps, and the like. As used herein, “shoot” refers to the above ground parts including the leaves and stems.

The term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ.

Any plant (or groupings of plants, for example, into a genus or higher order classification) can be employed in practicing the present invention including angiosperms or gymnosperms, monocots or dicots.

Exemplary plants include, but are not limited to corn (Zea mays), canola (Brassica napus, Brassica rapa ssp.), alfalfa (Medicago saliva), rice (Oryza sativa, including without limitation Indica and/or Japonica varieties), rape (Brassica napus), rye (Secale cereale), sorghum (Sorghum bicolor, Sorghum vulgare), sunflower (Helianthus annus), wheat (Triticum aestivum), soybean (Glycine max), tobacco (Nicotiana tobacum), potato (Solanum tuberosum), peanuts (Arachis hypogaea), cotton (Gossypium hirsutum), sweet potato (Ipomoea batatus), cassava (Manihot esculenta), coffee (Cofea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), apple (Malus pumila), blackberry (Rubus), strawberry (Fragaria), walnut (Juglans regia), grape (Vitis vinifera), apricot (Prunus armeniaca), cherry (Prunus), peach (Prunus persica), plum (Prunus domestica), pear (Pyrus communis), watermelon (Citrullus vulgaris). duckweed (Lemna), oats (Avena sativa), barley (Hordium vulgare), vegetables, ornamentals, conifers, and turfgrasses (e.g., for ornamental, recreational or forage purposes), and biomass grasses (e.g., switchgrass and miscanthus).

Vegetables include Solanaceous species (e.g., tomatoes; Lycopersicon esculentum), lettuce (e.g., Lactuea sativa), carrots (Caucus carota), cauliflower (Brassica oleracea), celery (apium graveolens), eggplant (Solanum melongena), asparagus (Asparagus officinalis), ochra (Abelmoschus esculentus), green beans (Phaseolus vulgaris), lima beans (Phaseolus limensis), peas (Lathyrus spp.), members of the genus Cucurbita such as Hubbard squash (C. Hubbard), Butternut squash (C. moschata), Zucchini (C. pepo), Crookneck squash (C. crookneck), C. argyrosperma, C. argyrosperma ssp sororia, C. digitata, C. ecuadorensis, C. foetidissima, C. lundelliana, and C. martinezii, and members of the genus Cucumis such as cucumber (Cucumis sativus), cantaloupe (C. cantalupensis), and musk melon (C. melo).

Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (dianthus caryophyllus), poinsettia (Euphorbia pulcherima), and chrysanthemum.

Conifers, which may be employed in practicing the present invention, include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis).

Turfgrass include but are not limited to zoysiagrasses, bentgrasses, fescue grasses, bluegrasses, St. Augustinegrasses, bermudagrasses, bufallograsses, ryegrasses, and orchardgrasses.

Also included are plants that serve primarily as laboratory models, e.g., Arabidopsis thaliana.

### II. METHODS OF INCREASING TOLERANCE TO HIGH TEMPERATURE OR HEAT AND/OR AMINO ACID CONTENT

The invention provides methods of introducing a glutamine synthetase 1;2 (GS1;2; E.C. 6.3.1.2), a glutamate decarboxylase 3 (GAD3; E.C. 4.1.1.15), a class I glutamine amidotransferase (GAT1; E.C. 2.6.5.2), a MYB55 polypeptide, or any combination thereof into a plant material, e.g., a plant, plant part (including callus) or plant cell (e.g., to express the GS1;2, GAD3, GAT1 and/or MYB55 polypeptide in the plant material). In representative embodiments, the method comprises transforming the plant material with a nucleic acid (e.g., isolated nucleic acid), expression cassette, or vector as described herein encoding the GS1;2, GAD3, GAT1 and/or MYB55 polypeptide. The plant can be transiently or stably transformed.

As one aspect, the invention encompasses a method of increasing tolerance to heat stress or high temperature in a transgenic plant, plant part or plant cell, the method comprising introducing one or more nucleic acids (e.g., isolated nucleic acids) encoding (i) a GS1;2, (ii) a GAD3, (iii) a GAT1, (iv) a MYB55 polypeptide or any combination thereof into the plant, plant part or plant cell to produce a transgenic plant, plant part or plant cell that expresses the one or more nucleic acids to produce GS1;2, GAD3, GAT1, MYB55 polypeptide or any combination thereof (e.g., in an amount effective to increase tolerance to heat stress or high temperature), thereby resulting in an increased tolerance to heat stress or high temperature in the transgenic plant, plant part or plant cell as compared with a control plant, plant part or plant cell. The plant, plant part or plant cell can be transiently or stably transformed.

The increased tolerance to heat stress or high temperature can be assessed with respect to any relevant control plant, e.g., a plant, plant part or plant cell that has not been transformed with the one or more nucleic acids according to the methods of the invention. The control plant is generally matched for species, variety, age, and the like and is optionally subjected to the same growing conditions, e.g., temperature, soil, sunlight, pH, water, and the like. The selection of a suitable control plant is routine for those skilled in the art.

In representative embodiments, the one or more nucleic acids encode GS1;2, GAD3, and GAT1. The enzymes can be encoded by one or more than one (e.g., two or three) isolated nucleic acids. For example, each enzyme can be encoded by a different nucleic acid. Alternatively, one nucleic acid can encode two or all three enzymes. Optionally, the method can further comprise introducing a nucleic acid (e.g., an isolated nucleic acid) encoding a MYB55 polypeptide into the plant, plant part or plant cell. The MYB55 polypeptide can be encoded by a separate nucleic acid or can be encoded by the same nucleic acid as one or more of the GS1;2, GAD3 and/or GAT1 enzymes.

In particular embodiments, the method comprises: (a) introducing the one or more nucleic acids (e.g., isolated nucleic acids) encoding (i) a GS1;2, (ii) a GAD3, (iii) a GAT1, (iv) a MYB55 polypeptide or any combination thereof into a plant cell (including a callus cell) to produce a transgenic plant cell; and (b) regenerating a transgenic plant from the transgenic plant cell of (a), optionally wherein the transgenic plant comprises in its genome the one or more nucleic acids and, as a further option, has increased tolerance to heat stress or high temperature as compared with a control plant (e.g., expresses GS1;2, GAD3, GAT1 and/or a MYB55 polypeptide in an amount effective to increase tolerance to heat stress or high temperature in the plant).

In additional embodiments, the method comprises: (a) introducing the one or more nucleic acids (e.g., isolated nucleic acids) encoding (i) a GS1;2, (ii) a GAD3, (iii) a GAT1, (iv) a MYB55 polypeptide or any combination thereof into a plant cell (including a callus cell) to produce a transgenic plant cell; and (b) regenerating a transgenic plant from the transgenic plant cell of (a), optionally wherein the transgenic plant comprises in its genome the one or more nucleic acids; and (c) selecting from a plurality of the transgenic plants of (b) a transgenic plant having increased tolerance to heat stress or high temperature (e.g., the transgenic plant expresses GS1;2, GAD3 GAT1 and/or a MYB55 polypeptide in an amount effective to increase tolerance to heat stress or high temperature in the plant).

Optionally, the methods of the invention can further comprise exposing the plant, plant part or plant cell to heat stress or high temperature, e.g., during the vegetative stage of growth. By exposing a plant, plant part or plant cell to heat stress or high temperature during the vegetative stage of growth it is meant that the plant, plant part or plant cell is subjected to the heat stress or high temperature for all or a portion of the vegetative stage of growth.

Further, in embodiments of the invention, the methods of the invention result in an increased yield as compared with a suitable control, e.g., an increase in plant height, plant biomass (e.g., dry biomass) and/or seed as compared with a plant that was not produced according to the methods of the invention. Those skilled in the art will appreciate that there may still be a reduced yield as compared with a plant, plant part or plant cell that was not exposed to the heat stress or high temperature.

In representative embodiments, a method of increasing tolerance of a plant to heat stress or high temperature comprises reducing an adverse effect on plant functions, development and/or performance as a result of heat stress or high temperature, e.g., reduced cell division, size (e.g., plant height) and/or number of plants and/or parts thereof and/or impairment in an agronomic trait such as reduced yield, fruit drop, fruit size and/or number, seed size and/or number, reduced quality of produce due to appearance and/or texture and/or increased flower abortion.

The invention also contemplates a method of increasing amino acid content of a transgenic plant, plant part or plant cell, the method comprising introducing a nucleic acid (e.g., an isolated nucleic acid) encoding a MYB55 polypeptide into the plant, plant part or plant cell to produce a transgenic plant, plant part or plant cell that expresses the nucleic acid to produce the MYB55 polypeptide (e.g., in an amount effective to increase amino acid content), thereby resulting in an increased amino acid content in the transgenic plant, plant part or plant cell as compared with a control plant, plant part or plant cell. The plant, plant part or plant cell can be transiently or stably transformed.

The increased amino acid content can be assessed with respect to any relevant control plant, e.g., a plant, plant part or plant cell that has not been transformed a nucleic acid encoding a MYB55 polypeptide according to the methods of the invention. The control plant is generally matched for species, variety, age, and the like and is subjected to the same growing conditions, e.g., temperature, soil, sunlight, pH, water, and the like. The selection of a suitable control plant is routine for those skilled in the art.

In particular embodiments, the method comprises: (a) introducing the nucleic acid (e.g., isolated nucleic acid) encoding a MYB55 polypeptide into a plant cell (including a callus cell) to produce a transgenic plant cell; and (b) regenerating a transgenic plant from the transgenic plant cell of (a), optionally wherein the transgenic plant comprises in its genome the nucleic acid and, as a further option, has increased amino acid content as compared with a control plant (e.g., expresses the MYB55 polypeptide in an amount effective to increase amino acid content in the plant).

In additional embodiments, the method comprises: (a) introducing the nucleic acid (e.g., isolated nucleic acid) encoding a MYB55 polypeptide into a plant cell (including a callus cell) to produce a transgenic plant cell; and (b) regenerating a transgenic plant from the transgenic plant cell of (a), optionally wherein the transgenic plant comprises in its genome the nucleic acid; and (c) selecting from a plurality of the transgenic plants of (b) a transgenic plant having increased amino acid content (e.g., the transgenic plant expresses MYB55 in an amount effective to increase amino acid content in the plant).

Optionally, the methods of the invention further comprise exposing the plant, plant part or plant cell to heat stress or high temperature, e.g., during the vegetative stage of growth.

In representative embodiments, the content of total amino acids is increased in the plant, plant part or plant cell. In additional embodiments, the content of one or more individual amino acids is increased. In particular embodiments, the glutamic acid, arginine, GABA and/or proline content is increased in the plant, plant part or plant cell.

The increased amino acid content can be observed with respect to the total plant biomass and/or can be observed within one or more plant parts or tissues, e.g., leaf, leaf sheath, root, or any combination thereof. In representative embodiments, the increased amino acid content can be present in a transgenic plant, plant part or plant tissue that is regenerated from a transgenic plant cell produced according to the methods of the invention. Optionally, the content of one or more particular amino acids may be increased in one plant part or tissue and the content of a different amino acid or combination of amino acids is increased in another plant part or tissue.

The invention also contemplates the production of progeny plants that comprise a nucleic acid (e.g., an isolated nucleic acid) encoding a GS1;2, a GAD3, a GAT1, a MYB55 polypeptide or any combination thereof. In embodiments of the invention, the method further comprises obtaining a progeny plant derived from the transgenic plant (e.g., by sexual reproduction or vegetative propagation). Optionally the progeny plant comprises in its genome an isolated nucleic acid encoding a GS1;2, a GAD3, a GAT1, a MYB55 polypeptide or any combination thereof and has increased tolerance to heat stress or high temperature as compared with a control plant (e.g., expresses the GS1;2, GAD3, GAT1, MYB55 polypeptide or any combination thereof in an amount effective to increase tolerance to heat stress or high temperature in the plant). In additional embodiments, the progeny plant comprises in its genome an isolated nucleic acid encoding a GS1;2, a GAD3, a GAT1, a MYB55 polypeptide or any combination thereof and has increased amino acid content as compared with a control plant (e.g., expresses the GS1;2, GAD3, GAT1, MYB55 polypeptide or any combination thereof in an amount effective to increase amino acid content in the plant).

To illustrate, in one embodiment, the invention provides a method of producing a progeny plant, the method comprising (a) crossing the transgenic plant comprising the one or more nucleic acids (e.g., isolated nucleic acids) encoding a GS1;2, a GAD3, a GAT1, a MYB55 polypeptide or any combination thereof with itself or another plant to produce seed comprising the one or more nucleic acids; and (b) growing a progeny plant from the seed to produce a transgenic plant, optionally wherein the progeny plant comprises in its genome the one or more nucleic acids encoding a GS1;2, a GAD3, a GAT1, a MYB55 polypeptide or any combination thereof and has increased tolerance to heat stress or high temperature and/or has an increased amino acid content as compared with a control plant (e.g., expresses the GS1;2, GAD3, GAT1, MYB55 polypeptide or any combination thereof in an amount effective to increase tolerance to heat stress or high temperature in the plant). In additional embodiments, the method can further comprise (c) crossing the progeny plant with itself or another plant and (d) repeating steps (b) and (c) for an additional 0-7 (e.g., 0, 1, 2, 3, 4, 5, 6 or 7 and any range thereof) generations to produce a plant, optionally wherein the plant comprises in its genome the one or more nucleic acids GS1;2, a GAD3, a GAT1, a MYB55 polypeptide or any combination thereof and has increased tolerance to heat stress or high temperature and/or has an increased amino acid content (e.g., expresses the GS1;2, GAD3, GAT1, MYB55 polypeptide or combination thereof in an amount effective to increase tolerance to heat stress or high temperature and/or amino acid content in the plant).

The terms “GS1;2”, “GAD3” and “GAT1” are intended broadly, and encompass any “GS1;2”, “GAD3” and/or “GAT1” now known or later discovered including biologically active equivalents thereof and biologically active fragments of full-length GS1;2, GAD3 and GAT1 polypeptides and equivalents of such fragments. The terms “GS1;2”, “GAD3” and “GAT1” also include modifications (e.g., deletions and/or truncations) of a naturally occurring polypeptide or an equivalent thereof that have a substantially similar or identical amino acid sequence to a naturally occurring polypeptide and that have enzymatic activity and/or increase tolerance to heat stress or high temperature and/or increase amino acid content in a plant, plant part or plant cell. The GS1;2, GAD3 and GAT1 can be from any species of origin (e.g., a plant species including without limitation rice [including indica and/or japonica varieties], wheat, barley, maize, sorghum, oats, rye, sugar cane, Arabidopsis and the like), and the terms “GS1;2,” “GAD3” and “GAT1’ also include naturally occurring allelic variations, isoforms, splice variants and the like. The GS1;2, GAD3 and GAT1 can further be wholly or partially synthetic. These enzymes are well-known in the art and have previously been described in a number of plant species.

For example, it is known that plants have multiple isozymes of class 2 glutamine synthetase. The GS1:2 isozyme is a cytosolic form, and is involved in converting glutamine into glutamic acid and represents one of the early steps in amino acids biosynthesis. The enzyme is a homo-octomer composed of eight identical subunits separated into two face-to-face rings. ATP binds to the top of the active site near a cation binding site, whereas glutamate binds near a second cation binding site at the bottom of the active site. Ammonium, rather than ammonia, binds to active site because the binding site is polar and exposed to solvent. The nucleotide and amino acid sequences of a number of GS1;2 are known, e.g., in rice (GenBank Accession Nos. NP_{—}001051067 and P14654 [amino acid] and AB180688.1 and NM_{—}001057602 [nucleotide]), maize (GenBank Accession Nos. NP_{—}001105443 and BAA03433 [amino acid] and NM_{—}001111973 (nucleotide), soybean (GenBank Accession Nos. NP_{—}001242332 [amino acid] and NM_{—}001255403 [nucleotide]), Arabidopsis (GenBank Accession Nos. NP_{—}176794 [amino acid] and NM_{—}105291 [nucleotide]), sugar cane (GenBank Accession Nos. AAW21275 [amino acid] and AY835455 [nucleotide]), and the like. A number of functional domains have been identified in GS1;2 proteins including the beta-Grasp domain and catalytic domain. The crystal structure of the maize GS1a is described in Unno et al. (2006, J. Biol. Chem. 281:29287-29296; see also, RCSB Protein Data Bank ID 2D3C).

GAT1 is also known as carbamoyl phosphate synthetase and is involved in the first committed step in arginine biosynthesis in prokaryotes and eukaryotes. The nucleotide and amino acid sequences of a number of GAT1 are known, e.g., in rice (GenBank Accession Nos. BAD08105.1 and NP_{—}001047880 [amino acid] and NM_{—}001054415 [nucleotide]), maize (GenBank Accession Nos. NP_{—}001132055 [amino acid] and NM_{—}001138583 [nucleotide]), soybean (GenBank Accession Nos. XP_{—}003525104 [amino acid] and XM_{—}003525056 [nucleotide]), Arabidopsis (GenBank Accession Nos. NP_{—}566824 [amino acid] and NM_{—}113690 [nucleotide]), and the like. A number of functional domains have been identified in GAT1 proteins including the catalytic (active) site, which is defined by a conserved catalytic triad of cysteine, histidine and glutamate. The crystal structure of GAT1 from a number of bacteria have been described including the GAT1 From T. thermophilus (RCSB Protein Data Bank ID 2YWD) and P. horikoshii (RCSB Protein Data Bank ID 2D7J).

GAD3 is involved in converting L-glutamic acid into GABA. The nucleotide and amino acid sequences of a number of GAD3 are known, e.g., in rice (GenBank Accession Nos. AAO59316 [amino acid] and AY187941 [nucleotide]), soybean (GenBank Accession Nos. BAF80895 [amino acid] and AB240965 [nucleotide]), Arabidopsis (GenBank Accession Nos. NP_{—}178309 [amino acid] and NM_{—}126261 [nucleotide]), and the like. A number of functional domains have been identified in GAD3 proteins including the pyridoxal 5′-phosphate (cofactor) binding site and the catalytic (active) site. The crystal structure of GAD3 has been resolved from bacteria, including E. coli (RCSB Protein Data Bank ID 3FZ7 and 3FZ6).

Generally, the GS1;2, GAT1 and GAD3 used according to the methods of the invention have enzymatic activity and/or increase tolerance to heat stress or high temperature when expressed in a plant, plant part or plant cell.

Unless indicated otherwise, the GS1;2, GAT1 and GAD3 polypeptides include fusion proteins comprising a GS1;2, GAT1 or GAD3 polypeptide of the invention. For example, it may be useful to express these polypeptides as a fusion protein that can be detected by a commercially available antibody (e.g., a FLAG motif) or as a fusion protein that can otherwise be more easily detected or purified (e.g., by addition of a poly-His tail). Additionally, fusion proteins that enhance the stability of the protein can be produced, e.g., fusion proteins comprising maltose binding protein (MBP) or glutathione-S-transferase. As another alternative, the fusion protein can comprise a reporter molecule.

The terms “GS1;2”, “GAT1” and “GAD3” encompass full-length polypeptides and biologically active fragments thereof as well as biologically active equivalents of either of the foregoing that have substantially similar or substantially identical amino acid sequences (e.g., at least about 60%, 70%, 75%, 80%, 85%, 90%, 95%, 97%, 98%, 99% or even 100% amino acid sequence similarity or identity), where the biologically active fragment or biologically active equivalent retains one or more of the biological activities of the native enzyme.

It will further be understood that naturally occurring GS1;2, GAT1 and GAD3 will typically tolerate substitutions in the amino acid sequence and substantially retain biological activity. To routinely identify biologically active polypeptides other than naturally occurring GS1;2, GAT1 and GAD3, amino acid substitutions may be based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are made in the amino acid sequence encoding the GS1;2, GAT1 or GAD3 polypeptide.

In representative embodiments, a biologically active GS1;2, GAT1 or GAD3 (including equivalents and fragments thereof) is catalytically active and increases tolerance to heat stress or high temperature in a plant, plant part of plant cell. Methods of assessing the enzymatic activity for these enzymes are known in the art, as are methods of measuring tolerance of a plant, plant part or plant cell to heat stress or high temperature. Those skilled in the art will be able to routinely identify biologically active equivalents of these enzymes and biologically active fragments and biologically active equivalents thereof using the extensive knowledge that exists in the art and the teachings in the present application.

The length of the GS1;2, GAT1 or GAD3 fragment (e.g., biologically active fragment) is not critical. Illustrative fragments comprise at least about 50, 60, 70, 80, 90, 100, 110, 120, 130, 140, 150, 160, 170, 180, 190, 200, 225, 250, 275, 300, 325, 350, 375, 400, 425, 450, 475 or 500 amino acids (optionally, contiguous amino acids) of a full-length polypeptide.

In representative embodiments, a biologically active equivalent of a GS1;2, a biologically active fragment of a GS1;2, or a biologically active equivalent thereof, comprises the catalytic domain and/or the beta-Grasp domain, and optionally any sequence variability occurs outside of this region(s). In embodiments, the biologically active equivalent, biologically active fragment or biologically equivalent of the fragment is a cytosolic polypeptide.

In representative embodiments, a biologically active equivalent of a GS1;2, a fragment of a GAT1, or a biologically active equivalent thereof, comprises the catalytic site, any sequence variability occurs outside this region. In embodiments, the catalytic triad (cysteine, histidine and glutamate) is conserved.

In representative embodiments, a biologically active equivalent of a GAD3, a fragment of a GAD3, or a biologically active equivalent thereof, comprises the pyridoxal 5′-phosphate (cofactor) binding site and/or the catalytic site, and optionally any sequence variability occurs outside of this region(s).

In additional embodiments, the GS1;2, GAT1 and GAD3 polypeptides are full-length polypeptides and exclude biologically active fragments.

The invention further provides nucleic acids encoding GS1; GAD3 and GAT1 polypeptides.

The term “MYB55 polypeptide” is intended broadly and encompasses naturally occurring MYB55 polypeptides now known or later identified and equivalents (including fragments and equivalents thereof) thereof that increase tolerance to heat stress or high temperature and/or increase amino acid content in a plant. The term “MYB55” polypeptide also includes modifications (e.g., deletions and/or truncations) of a naturally occurring MYB55 polypeptide or an equivalent thereof that has a substantially similar or identical amino acid sequence to a naturally occurring MYB55 polypeptide and that increase tolerance to heat stress or high temperature and/or increase amino acid content in a plant, plant part or plant cell. Further, the MYB55 polypeptide can be from any plant species of origin (e.g., rice [including indica and/or japonica varieties], wheat, barley, maize, sorghum, oats, rye, sugar cane and the like), and the term “MYB55” also includes naturally occurring allelic variations, isoforms, splice variants and the like. The MYB55 polypeptide can further be wholly or partially synthetic.

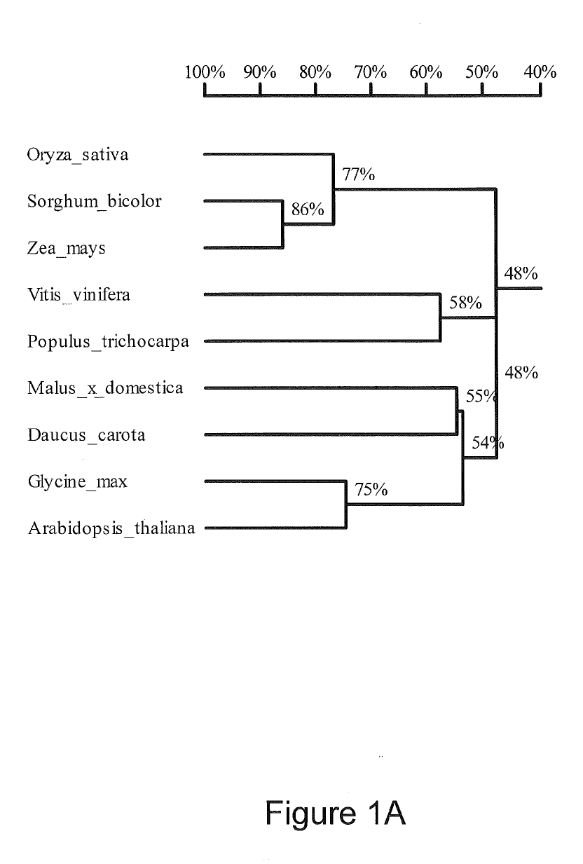

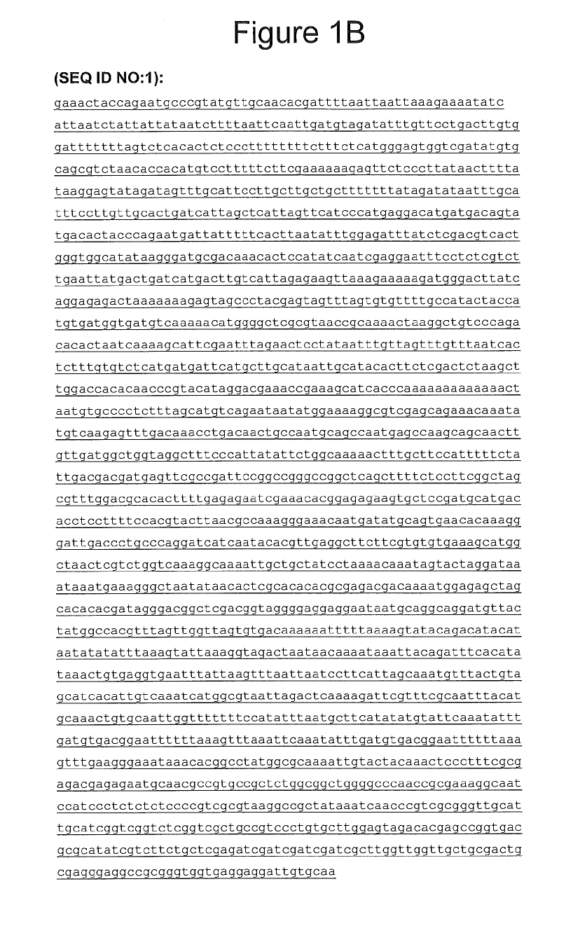

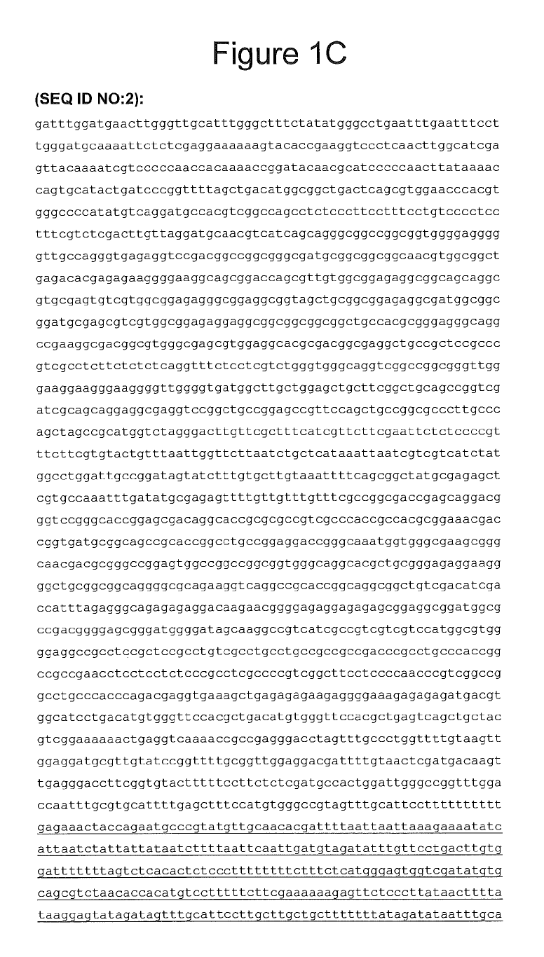

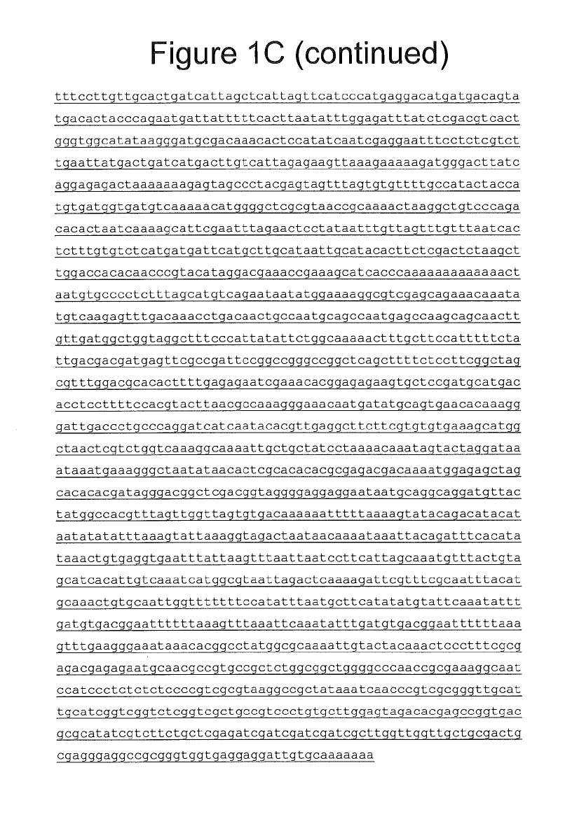

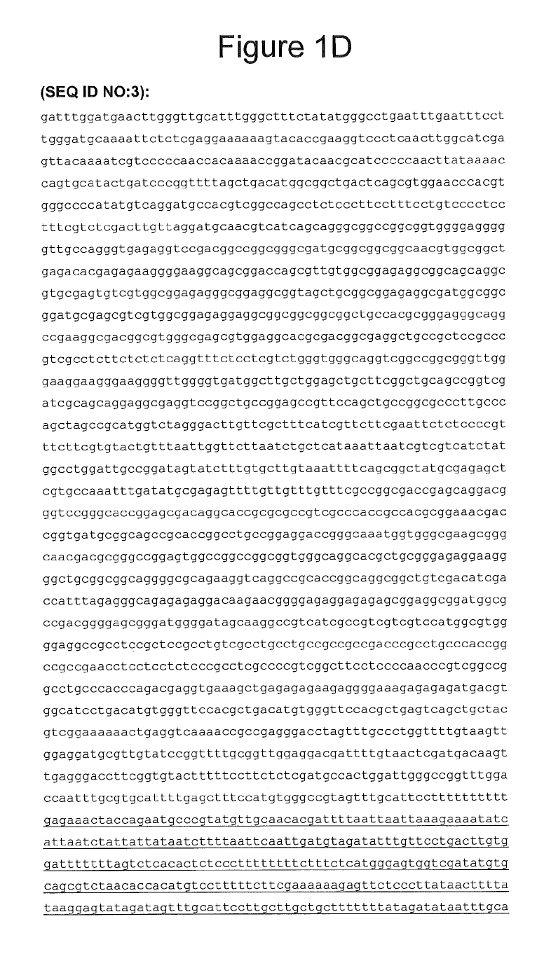

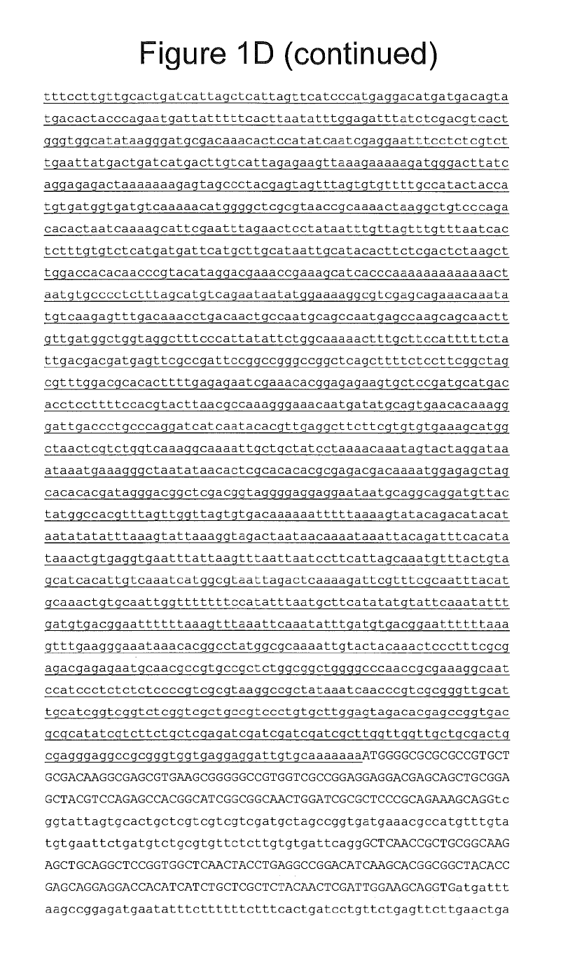

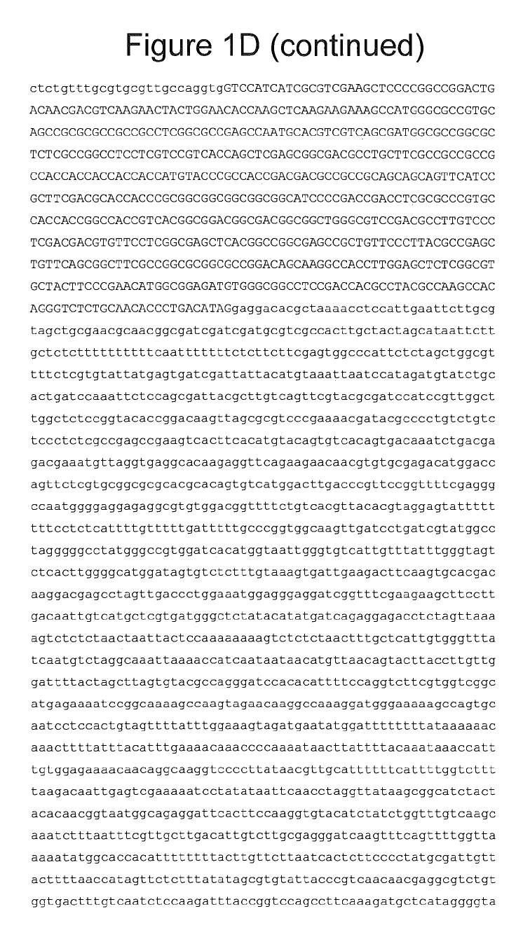

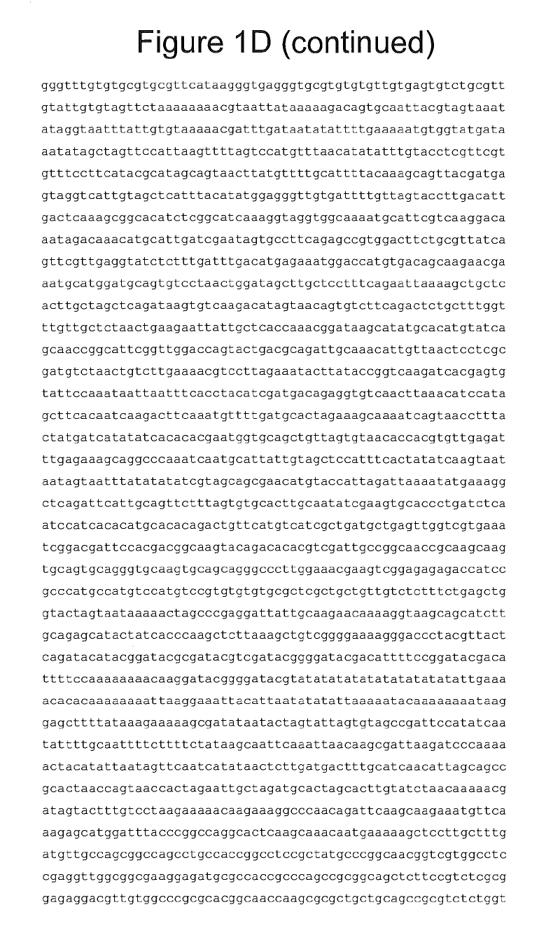

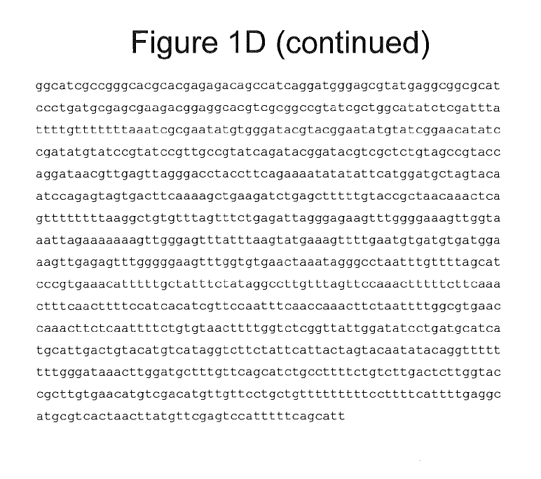

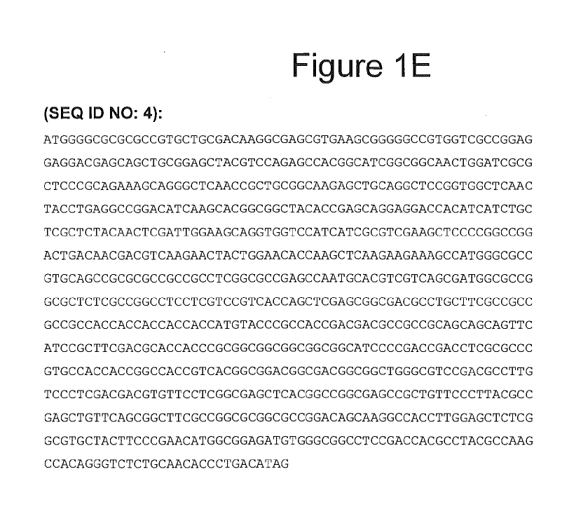

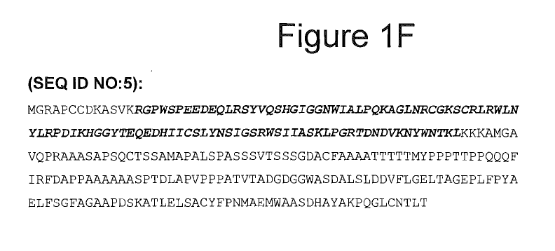

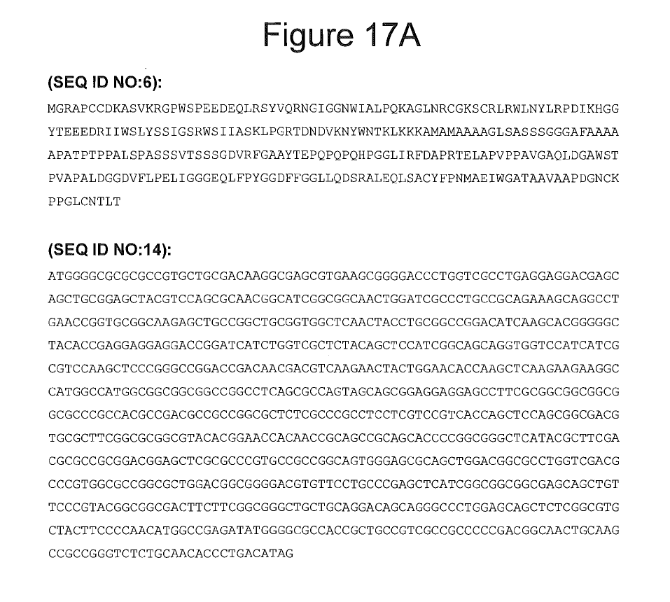

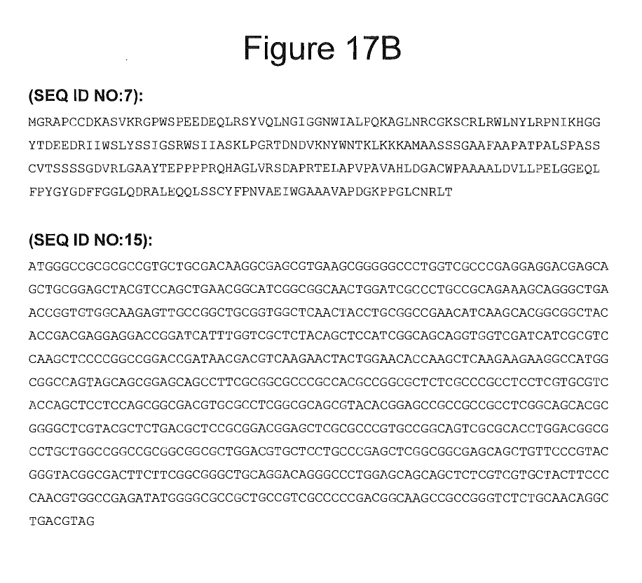

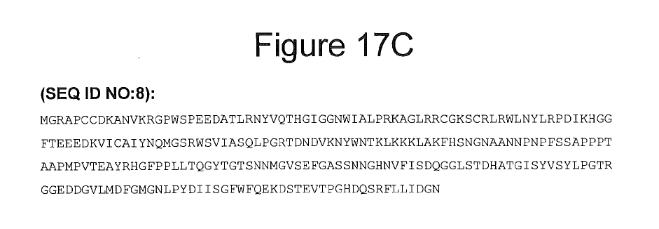

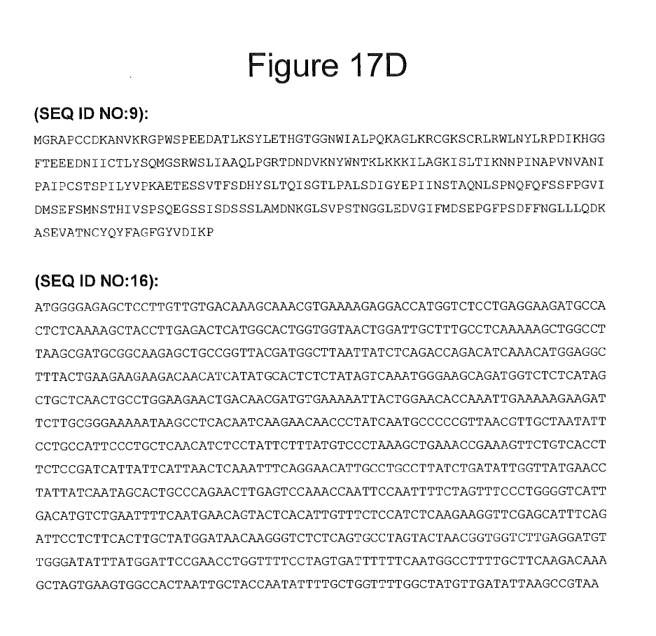

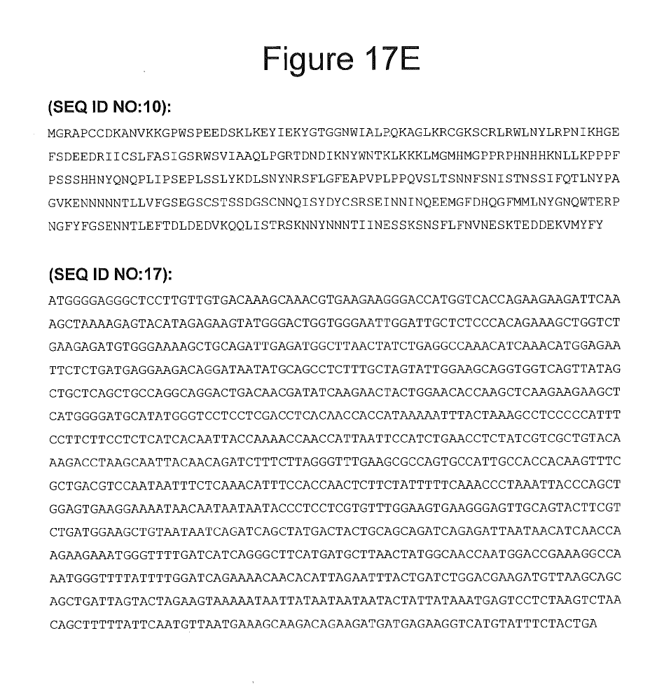

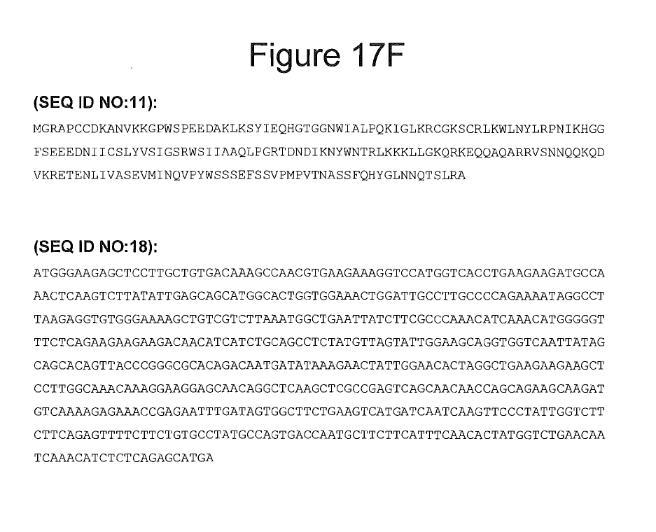

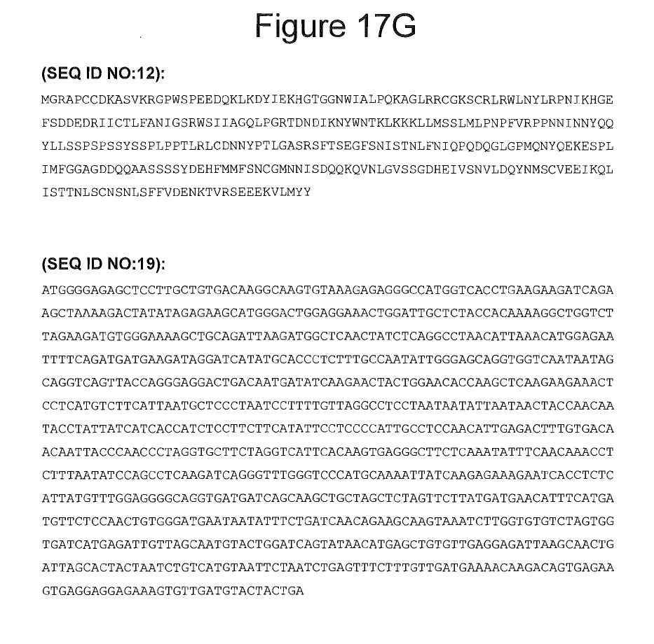

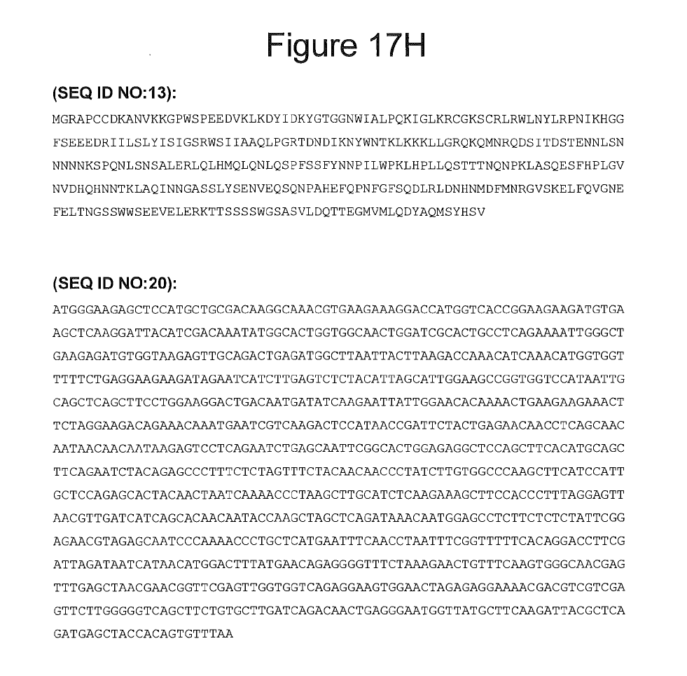

MYB55 polypeptides have been identified in a number of plant species including Oryza sativa (e.g., SEQ ID NO:5 [amino acid] and SEQ ID NO:4 and nucleotides 4062 to 5126 of SEQ ID NO:3 [nucleotide]), Sorghum bicolor(e.g., SEQ ID NO:6 [amino acid] and SEQ ID NO:14 [nucleotide]), Zea mays (e.g., SEQ ID NO:7 [amino acid] and SEQ ID NO:15 [nucleotide]), Vitis vinifera (e.g., SEQ ID NO:8 [amino acid]), Populus trichocarpa (e.g., SEQ ID NO:9 [amino acid] and SEQ ID NO:16 [nucleotide]), Malus x domestica (e.g., SEQ ID NO:10 [amino acid] and SEQ ID NO:17 [nucleotide]), Glycine max (e.g., SEQ ID NO:11 [amino acid] and SEQ ID NO:18 [nucleotide]), Daucus carota (e.g., SEQ ID NO:12 [amino acid] and SEQ ID NO:19 [nucleotide]), and Arabidopsis thaliana (e.g., SEQ ID NO:13 [amino acid] and SEQ ID NO:20 [nucleotide]). See also, FIGS. 1A, D-F, FIGS. 17A-H, and SEQ ID NO:311 of WO2010/200595. Homologs from other organisms, in particular other plants, can be routinely identified using methods known in the art. For example, PCR and other amplification techniques and hybridization techniques can be used to identify such homologs based on their sequence similarity to the sequences set forth herein.

Generally, the MYB55 polypeptides used according to the methods of the invention have transcription factor activity and/or increase tolerance to heat stress or high temperature and/or increase amino acid content when expressed in a plant, plant part or plant cell.

Unless indicated otherwise, MYB55 polypeptides include MYB55 fusion proteins comprising a MYB55 polypeptide of the invention. For example, it may be useful to express the MYB55 polypeptide as a fusion protein that can be detected by a commercially available antibody (e.g., a FLAG motif) or as a fusion protein that can otherwise be more easily detected or purified (e.g., by addition of a poly-His tail). Additionally, fusion proteins that enhance the stability of the protein can be produced, e.g., fusion proteins comprising maltose binding protein (MBP) or glutathione-S-transferase. As another alternative, the fusion protein can comprise a reporter molecule.

In particular embodiments, the MYB55 polypeptide comprises, consists essentially of, or consists of the amino acid sequence of any of SEQ ID NOs:5-13 or equivalents thereof (including fragments and equivalents thereof).

Equivalents of the MYB55 polypeptides of the invention encompass those that have substantial amino acid sequence identity or similarity, for example, at least about 60%, 70%, 75%, 80%, 85%, 90%, 95%, 97%, 98%, 99% or more amino acid sequence identity or similarity with the amino acid sequence of a naturally occurring MYB55 polypeptide (e.g., SEQ ID NOs:5-13) or a fragment thereof, optionally a biologically active fragment.

It will be understood that naturally occurring MYB55 will typically tolerate substitutions in the amino acid sequence and substantially retain biological activity. To routinely identify biologically active MYB55 polypeptides of the invention other than naturally occurring MYB55 polypeptides (e.g., SEQ ID NOs:5-13), amino acid substitutions may be based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are made in the amino acid sequence encoding the MYB55 polypeptide.

The MYB55 polypeptides of the present invention also encompass MYB55 polypeptide fragments and equivalents thereof that increase tolerance to heat or high temperature and/or increase amino acid content in a plant, and equivalents thereof. The length of the MYB55 fragment is not critical. Illustrative MYB55 polypeptide fragments comprise at least about 50, 60, 70, 80, 90, 100, 110, 120, 130, 140, 150, 160, 170, 180, 190, 200, 225, 250 or 275 amino acids (optionally, contiguous amino acids) of a full-length MYB55 polypeptide.

In representative embodiments, a biologically active equivalent of a MYB55 polypeptide, a biologically active fragment of a MYB55 polypeptide, or a biologically active equivalent thereof, comprises a DNA binding domain (e.g., a helix turn helix DNA binding domain) and, optionally, all variability occurs outside the DNA binding domain. In embodiments of the invention, a biologically active equivalent of a MYB55 polypeptide, a biologically active fragment of a MYB55 polypeptide, or a biologically active equivalent thereof comprises amino acids 14-113 of SEQ ID NO:5 or a sequence substantially similar thereto. In embodiments of the invention, a biologically active equivalent of a MYB55 polypeptide, a biologically active fragment of a MYB55 polypeptide, or a biologically active equivalent thereof comprises amino acids 38-62 of SEQ ID NO:5 or a sequence substantially similar thereto and/or amino acids 90-113 of SEQ ID NO:5 or a sequence substantially similar thereto. In embodiments of the invention, a biologically active equivalent of a MYB55 polypeptide, a biologically active fragment of a MYB55 polypeptide, or a biologically active equivalent thereof comprises amino acids 14-64 or a sequence substantially similar thereto. Optionally, these regions are conserved and all variability occurs outside these sequences.

In additional embodiments, the MYB55 polypeptide comprises, consists essentially of, or consists of an amino acid sequence selected from the group consisting of: (a) the amino acid sequence of any of SEQ ID NOs:5-13; (b) an amino acid sequence having at least about 60%, 70%, 75%, 80%, 85%, 90%, 95%, 97%, 98%, 99% or more amino acid sequence identity or similarity with the amino acid sequence of any of SEQ ID NOs:5-13, optionally wherein the MYB55 polypeptide is biologically active; and (c) a fragment comprising at least about 50, 60, 70, 80, 90, 100, 110, 120, 130, 140, 150, 160, 170, 180, 190, 200, 225, 250 or 275 amino acids (optionally, contiguous amino acids) of the amino acid sequence of (a) or (b) above, optionally wherein the fragment increases tolerance to heat stress or high temperature and/or increases amino acid content in a plant.

Nucleic acids encoding MYB55 polypeptides of the invention can be from any species of origin (e.g., plant species) or can be partially or completely synthetic. In representative embodiments, the nucleic acid encoding the MYB55 polypeptide is an isolated nucleic acid.

The invention also provides polynucleotides encoding the MYB55 polypeptides of the invention. In representative embodiments, the nucleotide sequence encoding the MYB55 polypeptide is a naturally occurring nucleotide sequence (e.g., SEQ ID NO:4, nucleotides 4062 to 5126 of SEQ ID NO:3, or SEQ ID NOs:14-20) or encodes a naturally occurring MYB55 polypeptide (e.g., SEQ ID NOs:5-13), or is a nucleotide sequence that has substantial nucleotide sequence identity thereto and which encodes a biologically active MYB55 polypeptide.

The invention further provides polynucleotides encoding the MYB55 polypeptides of the invention, wherein the polynucleotide hybridizes to the complete complement of a naturally occurring nucleotide sequence encoding a MYB55 polypeptide (e.g., SEQ ID NO:4 or SEQ ID NOs:14-20) or a nucleotide sequence that encodes a naturally occurring MYB55 polypeptide (e.g., SEQ ID NOs:5-13) under stringent hybridization conditions as known by those skilled in the art and encode a biologically active MYB55 polypeptide.

Further, it will be appreciated by those skilled in the art that there can be variability in the polynucleotides that encode the MYB55 polypeptides due to the degeneracy of the genetic code and/or the presence of Introns or other untranslated elements. The degeneracy of the genetic code, which allows different nucleotide sequences to code for the same protein, is well known in the art. Moreover, plant or species-preferred codons can be used in the polynucleotides encoding the MYB55 polypeptides, as is also well-known in the art.

In exemplary, but non-limiting, embodiments, the nucleic acid (e.g., recombinant or isolated nucleic acid) encoding a MYB55 polypeptide comprises, consists essentially of, or consists of a nucleotide sequence selected from the group consisting of: (a) a nucleotide sequence comprising the nucleotide sequence of any of SEQ ID NO:4 or SEQ ID NOs:14-20; (b) a nucleotide sequence comprising at least about 50, 75, 100, 125, 150, 175, 200, 225, 250, 275, 300, 325, 350, 375, 400, 425, 450, 475, 500 or 750 or more nucleotides (e.g., consecutive nucleotides) of the nucleotide sequence of any of SEQ ID NO:4 or SEQ ID NOs:14-20 (e.g., encoding a biologically active fragment of the MYB55 polypeptide of any of SEQ ID NOs:5-13); (c) a nucleotide sequence having at least about 60%, 70%, 75%, 80%, 85%, 90%, 95%, 97%, 98%, 99% or more sequence identity to the nucleotide sequence of (a) or (b); (d) a nucleotide sequence that hybridizes to the complete complement of the nucleotide sequence of (a) or (b) under stringent hybridization conditions; and (e) a nucleotide sequence that differs from the nucleotide sequence of any of (a) to (d) due to the degeneracy of the genetic code. In representative embodiments, the nucleotide sequence encodes a biologically active MYB55 polypeptide that increases tolerance to heat stress or high temperature and/or increases amino acid content in a plant.

In representative embodiments, the nucleotide sequence encodes the polypeptide of any of SEQ ID NOs:5-13, or an equivalent polypeptide having substantial amino acid sequence identity or similarity with any of SEQ ID NOs:5-13 (optionally, a biologically active equivalent that increases tolerance to heat stress or high temperature and/or increases amino acid content). In representative embodiments, the nucleotide sequence encodes an equivalent (optionally, a biologically active equivalent) of the polypeptide of any of SEQ ID NOs:5-13 and hybridizes to the complete complement of the nucleotide sequence of any of SEQ ID NO:4 or SEQ ID NOs:14-20 under stringent hybridization conditions.

In representative embodiments, the nucleotide sequence encodes the polypeptide of any of SEQ ID NOs:5-13. According to this embodiment, the nucleotide sequence can comprise, consist essentially of, or consist of any of SEQ ID NO:4 or SEQ ID NOs:14-20.

### III. EXPRESSION CASSETTES

In representative embodiments, a nucleic acid of the invention (e.g., an isolated nucleic acid) is comprised within an expression cassette and is in operable association with a promoter, e.g., a heterologous promoter. In embodiments, the nucleic acid is operably associated with the native promoter. In particular embodiments, the nucleic acid is operably associated with a heterologous promoter. The GS1;2, GAD3, GAT1 and/or MYB55 polypeptide can be comprised within one or more expression cassettes. For example, each of the polypeptides can be encoded by a different nucleic acid, which can be comprised within one or more expression cassettes (e.g., one, two, three or four). As one alternative, one nucleic acid can encode two or more of the polypeptides (e.g., two, three or four), which can be comprised within one or more expression cassettes (e.g., one, two or three).

The heterologous promoter can be any suitable promoter known in the art (including bacterial, yeast, fungal, insect, mammalian, and plant promoters). In particular embodiments, the promoter is a promoter for expression in plants. The selection of promoters suitable for use with the present invention can be made among many different types of promoters. Thus, the choice of promoter depends upon several factors, including, but not limited to, cell- or tissue-specific expression, desired expression level, efficiency, inducibility and/or selectability. For example, where expression in a specific tissue or organ is desired in addition to inducibility, a tissue-specific or tissue-preferred promoter can be used (e.g., a root specific or preferred promoter). In contrast, where expression in response to a stimulus is desired a promoter inducible by other stimuli or chemicals can be used. Where continuous expression is desired throughout the cells of a plant, a constitutive promoter can be chosen.

Non-limiting examples of constitutive promoters include cestrum virus promoter (cmp) (U.S. Pat. No. 7,166,770), an actin promoter (e.g., the rice actin 1 promoter; Wang et al. (1992) Mol. Cell. Biol. 12:3399-3406; as well as U.S. Pat. No. 5,641,876), Cauliflower Mosaic Virus (CaMV) 35S promoter (Odell et al. (1985) Nature 313:810-812), CaMV 19S promoter (Lawton et al. (1987) Plant Mol. Biol. 9:315-324), an opine synthetase promoter (e.g., nos, mas, ocs, etc.; (Ebert et al. (1987) Proc. Natl. Acad. Sci USA 84:5745-5749), Adh promoter (Walker et al. (1987) Proc. Natl. Acad. Sci. USA 84:6624-6629), sucrose synthase promoter (Yang & Russell (1990) Proc. Natl. Acad. Sci. USA 87:4144-4148), and a ubiquitin promoter.

Some non-limiting examples of tissue-specific promoters for use with the present invention include those derived from genes encoding seed storage proteins (e.g., β-conglycinin, cruciferin, napin phaseolin, etc.), zein or oil body proteins (such as oleosin), or proteins involved in fatty acid biosynthesis (including acyl carrier protein, stearoyl-ACP desaturase and fatty acid desaturases (fad 2-1)), and other nucleic acids expressed during embryo development (such as Bce4, see, e.g., Kridl et al. (1991) Seed Sci. Res. 1:209-219; as well as EP Patent No. 255378). Thus, the promoters associated with these tissue-specific nucleic acids can be used in the present invention.

Additional examples of tissue-specific promoters include, but are not limited to, the root-specific promoters RCc3 (Jeong et al. Plant Physiol. 153:185-197 (2010)) and RB7 (U.S. Pat. No. 5,459,252), the lectin promoter (Lindstrom et al. (1990) Der. Genet. 11:160-167; and Vodkin (1983) Prog. Clin. Biol. Res. 138:87-98), corn alcohol dehydrogenase 1 promoter (Dennis et al. (1984) Nucleic Acids Res. 12:3983-4000), S-adenosyl-L-methionine synthetase (SAMS) (Vander Mijnsbrugge et al. (1996) Plant and Cell Physiology, 37(8):1108-1115), corn light harvesting complex promoter (Bansal et al. (1992) Proc. Natl. Acad. Sci. USA 89:3654-3658), corn heat shock protein promoter (O'Dell et al. (1985) EMBO J. 5:451-458; and Rochester et al. (1986) EMBO J. 5:451-458), pea small subunit RuBP carboxylase promoter (Cashmore, “Nuclear genes encoding the small subunit of ribulose-I,5-bisphosphate carboxylase” pp. 29-39 In: Genetic Engineering of Plants, Hollaender ed., Plenum Press 1983; and Poulsen et al. (1986) Mol. Gen. Genet. 205:193-200)), Ti plasmid mannopine synthase promoter (Langridge et al. (1989) Proc. Natl. Acad. Sci. USA 86:3219-3223), Ti plasmid nopaline synthase promoter (Langridge et al. (1989), supra), petunia chalcone isomerase promoter (van Tunen et al. (1988) EMBO J. 7:1257-1263), bean glycine rich protein 1 promoter (Keller et al. (1989) Genes Dev. 3:1639-1646), truncated CaMV 35S promoter (O'Dell et al. (1985) Nature 313:810-812), potato patatin promoter (Wenzler et al. (1989) Plant Mol. Biol. 13:347-354), root cell promoter (Yamamoto et al. (1990) Nucleic Acids Res. 18:7449), maize zein promoter (Kriz et al. (1987) Mol. Gen. Genet. 207:90-98; Langridge et al. (1983) Cell 34:1015-1022; Reina et al. (1990) Nucleic Acids Res. 18:6425; Reina et al. (1990) Nucleic Acids Res. 18:7449; and Wandelt et al. (1989) Nucleic Acids Res. 17:2354), globulin-1 promoter (Belanger et al. (1991) Genetics 129:863-872), α-tubulin cab promoter (Sullivan et al. (1989) Mol. Gen. Genet. 215:431-440), PEPCase promoter (Hudspeth & Grula (1989) Plant Mol. Biol. 12:579-589), R gene complex-associated promoters (Chandler et al. (1989) Plant Cell 1:1175-1183), and chalcone synthase promoters (Franken et al. (1991) EMBQ J. 10:2605-2612). Particularly useful for seed-specific expression is the pea vicilin promoter (Czako et al. (1992) Mol. Gen. Genet. 235:33-40; as well as U.S. Pat. No. 5,625,136). Other useful promoters for expression in mature leaves are those that are switched on at the onset of senescence, such as the SAG promoter from Arabidopsis (Gan et al. (1995) Science 270:1986-1988).

In addition, promoters functional in plastids can be used. Non-limiting examples of such promoters include the bacteriophage T3 gene 9 5′ UTR and other promoters disclosed in U.S. Pat. No. 7,579,516. Other promoters useful with the present invention include but are not limited to the S-E9 small subunit RuBP carboxylase promoter and the Kunitz trypsin inhibitor gene promoter (Kti3).

Other tissue-specific or tissue-preferred promoters include inflorescence-specific or preferred and meristem-specific or -preferred promoters.

In some embodiments, inducible promoters can be used with the present invention. Examples of inducible promoters useable with the present invention include, but are not limited to, tetracycline repressor system promoters, Lac repressor system promoters, copper-inducible system promoters, salicylate-inducible system promoters (e.g., the PR1a system), glucocorticoid-inducible promoters (Aoyama et al. (1997) Plant J. 11:605-612), and ecdysone-inducible system promoters. Other non-limiting examples of inducible promoters include ABA- and turgor-inducible promoters, the auxin-binding protein gene promoter (Schwob et al. (1993) Plant J. 4:423-432), the UDP glucose flavonoid glycosyl-transferase promoter (Ralston et al. (1988) Genetics 119:185-197), the MPI proteinase inhibitor promoter (Cordero et al. (1994) Plant J. 6:141-150), the glyceraldehyde-3-phosphate dehydrogenase promoter (Kohler et al. (1995) Plant Mol. Biol. 29:1293-1298; Martinez et al. (1989) J. Mol. Biol. 208:551-565; and Quigley et al. (1989) J. Mol. Evol. 29:412-421) the benzene sulphonamide-inducible promoters (U.S. Pat. No. 5,364,780) and the glutathione S-transferase promoters. Likewise, one can use any appropriate inducible promoter described in Gatz (1996) Current Opinion Biotechnol. 7:168-172 and Gatz (1997) Annu. Rev. Plant Physiol. Plant Mol. Biol. 48:89-108.

Other suitable promoters include promoters from viruses that infect the host plant including, but not limited to, promoters isolated from Dasheen mosaic virus, Chlorella virus (e.g., the Chlorella virus adenine methyltransferase promoter; Mitra et al., (1994) Plant Molecular Biology 26:85), tomato spotted wilt virus, tobacco rattle virus, tobacco necrosis virus, tobacco ring spot virus, tomato ring spot virus, cucumber mosaic virus, peanut stump virus, alfalfa mosaic virus, and the like.

In additional embodiments, the promoter is induced by heat stress or high temperature, e.g., a MYB55 promoter. The term “MYB55 promoter” is intended to encompass the promoter sequences specifically disclosed herein (e.g., SEQ ID NO:1, nucleotides 1921 to 4061 of SEQ ID NO:2, nucleotides 2562 to 4061 of SEQ ID NO:2, or SEQ ID NO:2), and equivalents thereof (optionally, a biologically active equivalent) that have substantially identical nucleotide sequences to the MYB55 promoter sequences specifically disclosed herein, as well as fragments of a full-length MY/355 promoter (optionally, a biologically active fragment) and equivalents thereof (optionally, a biologically active equivalent) that have substantially identical nucleotide sequences to a fragment of the MYB55 promoter sequences specifically disclosed herein. The term “MYB55 promoter” includes sequences from rice as well as homologues from other plant species, including naturally occurring allelic variants, isoforms, splice variants, and the like, or can be partially or completely synthetic.

Homologues from other organisms, in particular other plants, can be identified using methods known in the art. For example, PCR and other amplification and hybridization techniques can be used to identify such homologues based on their sequence similarity to the sequences set forth herein.

Biological activities associated with the MYB55 promoter include, without limitation, the ability to control or regulate transcription of an operably associated coding sequence. Another non-limiting biological activity includes the ability to bind one or more transcription factors and/or RNA polymerase II. Other biological activities include without limitation the ability to be induced by heat stress, high temperature, ABA, MeJa and/or salicylic acid.

Thus, in exemplary embodiments, the isolated nucleic acid comprises, consists essentially of, or consists of SEQ ID NO:1, nucleotides 1921 to 4061 of SEQ ID NO:2, nucleotides 2562 to 4061 of SEQ ID NO:2, or SEQ ID NO:2 or an equivalent of any of the foregoing (optionally, a biologically active equivalent).

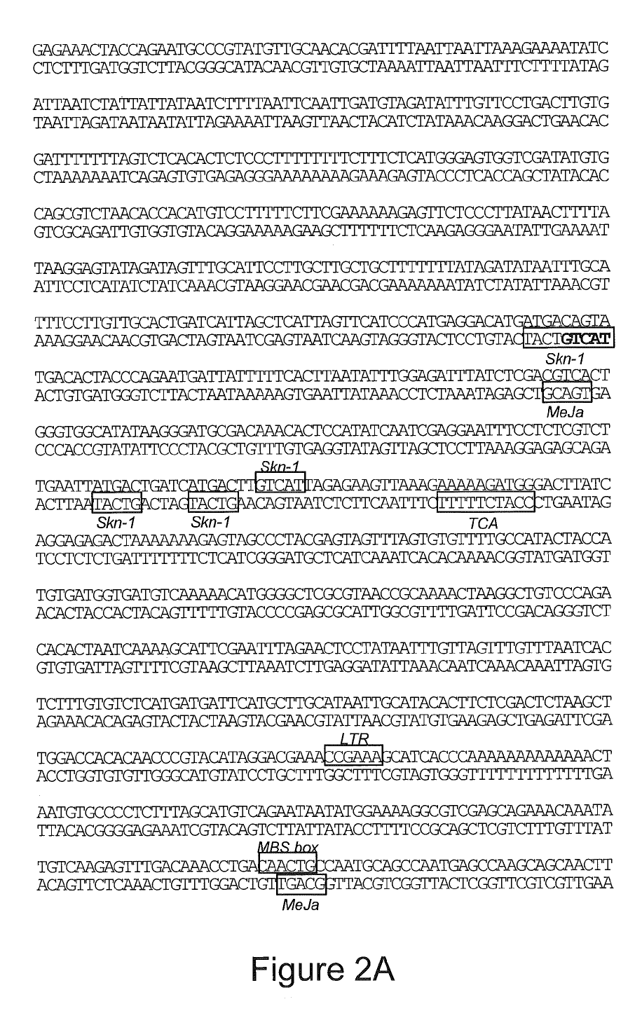

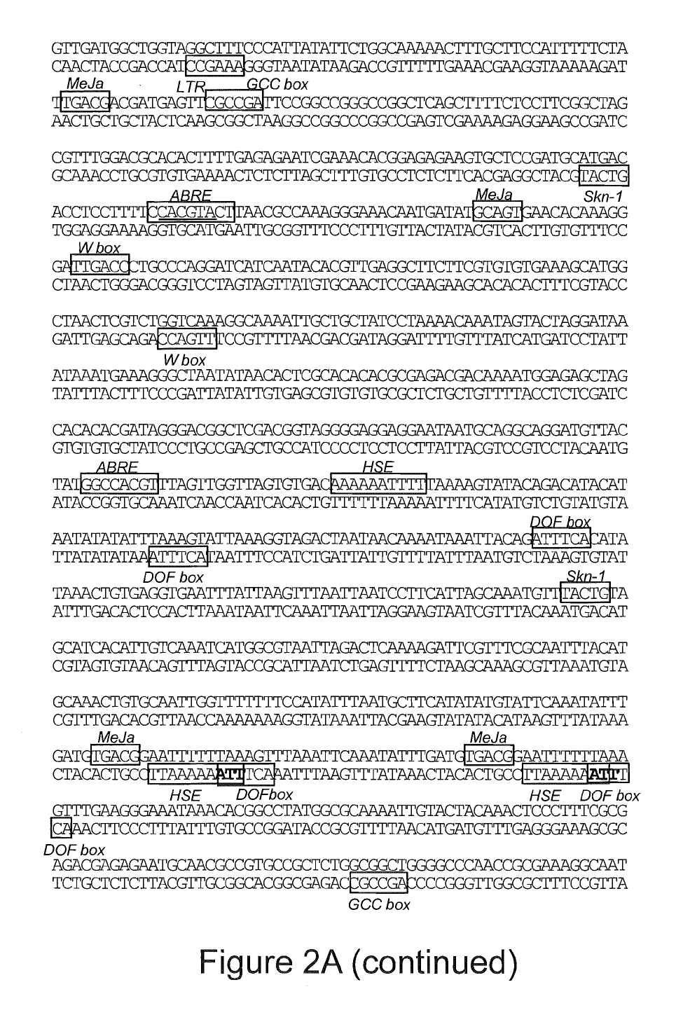

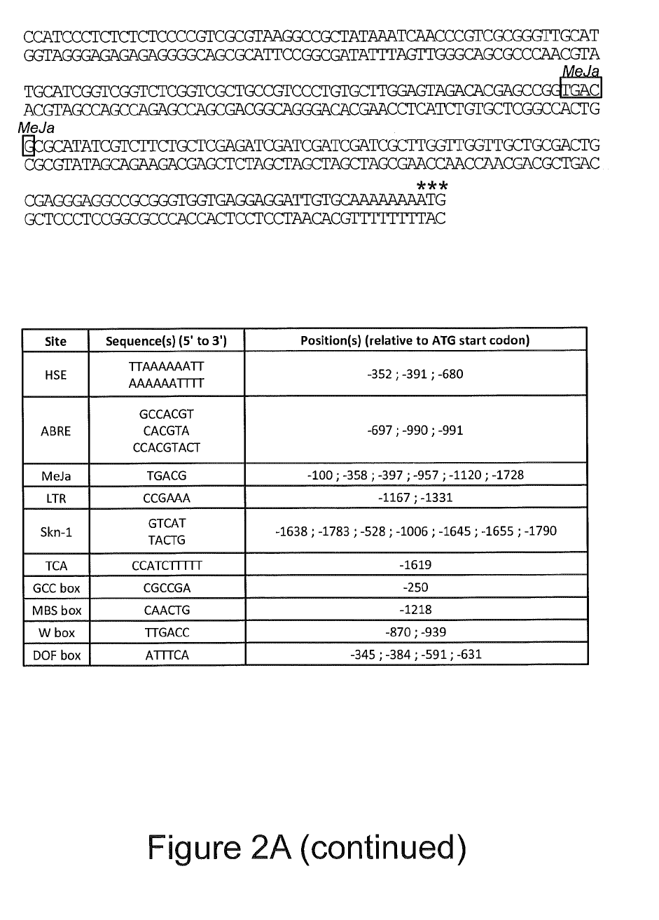

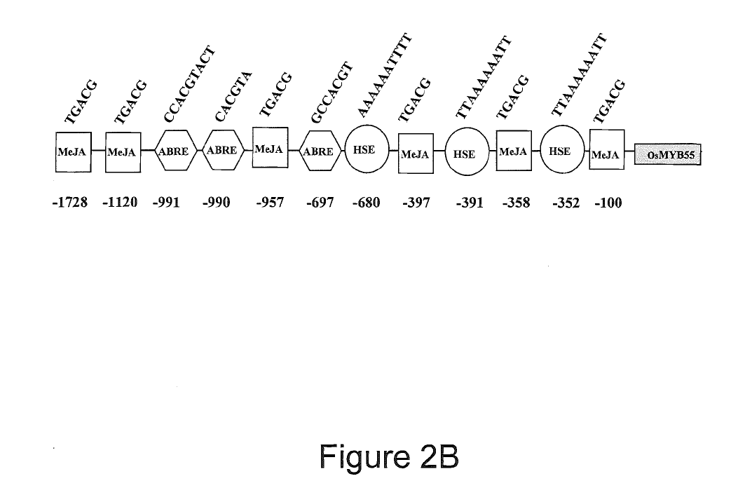

Equivalents of the MYB55 promoters of the invention encompass polynucleotides having substantial nucleotide sequence identity with the MYB55 promoter sequences specifically disclosed herein (e.g., SEQ ID NO:1, nucleotides 1921 to 4061 of SEQ ID NO:2, nucleotides 2562 to 4061 of SEQ ID NO:2, or SEQ ID NO:2) or fragments thereof, for example at least about 60%, 70%, 75%, 80%, 85%, 90%, 95%, 97%, 98% or 99% or more, and are optionally biologically active. In representative embodiments, there is no sequence variability in the TATA box, CAAT box, one or more of the Heat Shock Elements (HSE) (e.g., one, two or three), one or more of the ABA Responisve Elements (ABRE; e.g., one two or three), one or more of the Methyl Jasonate (MeJa) response elements (e.g., one, two, three, four or five), the Low Temperature Responsiveness (LTR) element, one or more of the DOF binding sites (“DOF box”; e.g., one, two or three), the MYB binding site (“MBS Box”), the AP-2 binding site (“GCC Box”, one or more of the WRKY binding sites (“W box”; e.g., one or two), one or more of the Skn-1 binding sites (e.g., one, two, three, four or five) and/or the TCA-element (see, e.g., the schematic in FIGS. 2A and 2B), i.e., these sequences are conserved and any sequence variability falls outside these regions.

The MYB55 promoters of the invention also include polynucleotides that hybridize to the complete complement of the MYB55 promoter sequences specifically disclosed herein (e.g., SEQ ID NO:1, nucleotides 1921 to 4061 of SEQ ID NO:2, nucleotides 2562 to 4061 of SEQ ID NO:2, or SEQ ID NO:2) or fragments thereof under stringent hybridization conditions as known by those skilled in the art and are optionally biologically active.

The MYB55 promoter sequences encompass fragments (optionally, biologically active fragments) of the MYB55 promoter sequences specifically disclosed herein (e.g., SEQ ID NO:1, nucleotides 1921 to 4061 of SEQ ID NO:2, nucleotides 2562 to 4061 of SEQ ID NO:2, or SEQ ID NO:2) and equivalents thereof. The length of the MYB55 promoter fragments is not critical. Illustrative fragments comprise at least and/or are greater than about 8, 10, 12, 15, 16, 17, 18, 19, 20, 25, 30, 35, 40, 45, 50, 75, 100, 125, 150, 175, 200, 250, 300, 350, 400, 450, 500, 600, 700, 800, 900, 1000, 1100, 1200, 1300, 1400, 1500, 1600, 1700, 1800, 1900, 2000, 2050, 2100, 2105, 2110, 2115, 2120, 2125, 2130, 2131, 2132, 2133, 2134, 2135, 2136, 2137, 2138 or 2139 or more nucleotides (optionally, contiguous nucleotides) of the full-length sequence.

In representative embodiments, the MYB55 promoter sequence comprises the TATA box sequence, the CAAT box sequence, one or more of the HSE elements (e.g., one, two or three), one or more of the ABRE elements (e.g., one two or three), one or more of the MeJa elements (e.g., one, two, three, four or five), the LTR element, one or more of the DOF binding sites (“DOF box”; e.g., one, two or three), the MYB binding site (“MBS Box”), the AP-2 binding site (“GCC Box”, one or more of the WRKY binding sites (“W box”; e.g., one or two), one or more of the Skn-1 binding sites (e.g., one, two, three, four or five) and/or the TCA-element (see, e.g., the schematic in FIGS. 2A and 2B), i.e., these sequences are conserved and any sequence variability falls outside these regions.

In embodiments of the invention, the nucleic acid comprising the MYB55 promoter does not include any of the MY855 coding region (e.g., nucleotides 4062 to 5126 of SEQ ID NO:3; FIG. 1D). In embodiments of the invention, the nucleotide sequence of interest does not encode a MYB55 polypeptide (e.g., SEQ ID NO:5; FIG. 1F). In embodiments of the invention, the nucleotide sequence of interest encodes a MYB55 polypeptide.

Accordingly, in representative embodiments, the invention provides a nucleic acid (e.g., a recombinant or isolated nucleic acid) comprising, consisting essentially of, or consisting of a nucleotide sequence selected from the group consisting of: (a) SEQ ID NO:1, nucleotides 1921 to 4061 of SEQ ID NO:2, nucleotides 2562 to 4061 of SEQ ID NO:2, or SEQ ID NO:2; (b) a nucleotide sequence comprising at least about 8, 10, 12, 15, 16, 17, 18, 19, 20, 25, 30, 35, 40, 45, 50, 75, 100, 125, 150, 175, 200, 250, 300, 350, 400, 450, 500, 600, 700, 800, 900, 1000, 1100, 1200, 1300, 1400, 1500, 1600, 1700, 1800, 1900, 2000, 2050, 2100, 2105, 2110, 2115, 2120, 2125, 2130, 2131, 2132, 2133, 2134, 2135, 2136, 2137, 2138 or 2139 or more nucleotides (optionally, contiguous nucleotides) of SEQ ID NO:1, nucleotides 1921 to 4061 of SEQ ID NO:2, nucleotides 2562 to 4061 of SEQ ID NO:2, or SEQ ID NO:2; (c) a nucleotide sequence that hybridizes to the complete complement of the nucleotide sequence of (a) or (b) under stringent hybridization conditions; and (d) a nucleotide sequence having at least about 60%, 70%, 75%, 80%, 85%, 90%, 95%, 97%, 98%, 99% sequence identity to the nucleotide sequences of any of (a) to (c). In representative embodiments, the nucleotide sequence is a biologically active promoter sequence (e.g., has promoter activity) and is optionally induced by heat stress, high temperature, ABA, salicylic acid and/or MeJa.

In embodiments of the invention, the nucleotide sequence comprises, consists essentially of, or consists of the nucleotide sequence of SEQ ID NO:1, nucleotides 1921 to 4061 of SEQ ID NO:2, nucleotides 2562 to 4061 of SEQ ID NO:2, or SEQ ID NO:2.

The expression cassettes of the invention may further comprise a transcriptional termination sequence. Any suitable termination sequence known in the art may be used in accordance with the present invention. The termination region may be native with the transcriptional initiation region, may be native with the nucleotide sequence of interest, or may be derived from another source. Convenient termination regions are available from the Ti-plasmid of A. tumefaciens, such as the octopine synthetase and nopaline synthetase termination regions. See also, Guerineau et al., Mol. Gen. Genet. 262, 141 (1991); Proudfoot, Cell 64, 671 (1991); Sanfacon et al., Genes Dev. 5,141 (1991); Mogen et al., Plant Cell 2, 1261 (1990); Munroe et al., Gene 91, 151 (1990); Ballas et al., Nucleic Acids Res. 17, 7891 (1989); and Joshi et al., Nucleic Acids Res. 15, 9627 (1987). Additional exemplary termination sequences are the pea RubP carboxylase small subunit termination sequence and the Cauliflower Mosaic Virus 35S termination sequence. Other suitable termination sequences will be apparent to those skilled in the art.

Further, in particular embodiments, the nucleotide sequence of interest is operably associated with a translational start site. The translational start site can be the native translational start site associated with the nucleotide sequence of interest, or can be any other suitable translational start codon.

In illustrative embodiments, the expression cassette includes in the 5′ to 3′ direction of transcription, a promoter, a nucleotide sequence of interest, and a transcriptional and translational termination region functional in plants.

Those skilled in the art will understand that the expression cassettes of the invention can further comprise enhancer elements and/or tissue preferred elements in combination with the promoter.

Further, in some embodiments, it is advantageous for the expression cassette to comprise a selectable marker gene for the selection of transformed cells. Suitable selectable marker genes include without limitation genes encoding antibiotic resistance, such as those encoding neomycin phosphotransferase II (NEO) and hygromycin phosphotransferase (HPT), as well as genes conferring resistance to herbicidal compounds. Herbicide resistance genes generally code for a modified target protein insensitive to the herbicide or for an enzyme that degrades or detoxifies the herbicide in the plant before it can act. See, DeBlock et al., EMBO J. 6, 2513 (1987); DeBlock et al., Plant Physiol. 91, 691 (1989); Fromm et al., BioTechnology 8, 833 (1990); Gordon-Kamm et al., Plant Cell 2, 603 (1990). For example, resistance to glyphosphate or sulfonylurea herbicides has been obtained using genes coding for the mutant target enzymes, 5-enolpyruvylshikimate-3-phosphate synthase (EPSPS) and acetolactate synthase (ALS). Resistance to glufosinate ammonium, boromoxynil, and 2,4-dichlorophenoxyacetate (2,4-D) have been obtained by using bacterial genes encoding phosphinothricin acetyltransferase, a nitrilase, or a 2,4-dichlorophenoxyacetate monooxygenase, which detoxify the respective herbicides.

Selectable marker genes that can be used according to the present invention further include, but are not limited to, genes encoding: neomycin phosphotransferase II (Fraley et al., CRC Critical Reviews in Plant Science 4, 1 (1986)); cyanamide hydratase (Maier-Greiner et al., Proc. Natl. Acad. Sci. USA 88, 4250 (1991)); aspartate kinase; dihydrodipicolinate synthase (Pert et al., BioTechnology 11, 715 (1993)); the bar gene (Toki et al., Plant Physiol. 100, 1503 (1992); Meagher et al., Crop Sci. 36, 1367 (1996)); tryptophane decarboxylase (Goddijn et al., Plant Mol. Biol. 22, 907 (1993)); neomycin phosphotransferase (NEO; Southern et al., J. Mol. Appl. Gen. 1, 327 (1982)); hygromycin phosphotransferase (HPT or HYG; Shimizu et al., Mol. Cell. Biol. 6, 1074 (1986)); dihydrofolate reductase (DHFR; Kwok et al., Proc. Natl. Acad. Sci. USA 83, 4552 (1986)); phosphinothricin acetyltransferase (DeBlock et al., EMBO J. 6, 2513 (1987)); 2,2-dichloropropionic acid dehalogenase (Buchanan-Wollatron et al., J. Cell. Biochem. 130, 330 (1989)); acetohydroxyacid synthase (U.S. Pat. No. 4,761,373 to Anderson et al.; Haughn et al., Mol. Gen. Genet. 221, 266 (1988)); 5-enolpyruvyl-shikimate-phosphate synthase (aroA; Comai et al., Nature 317, 741 (1985)); haloarylnitrilase (WO 87/04181 to Stalker et al.); acetyl-coenzyme A carboxylase (Parker et al., Plant Physiol. 92, 1220 (1990)); dihydropteroate synthase (sulI; Guerineau et al., Plant Mol. Biol. 15, 127 (1990)); and 32 kDa photosystem II polypeptide (psbA; Hirschberg et al., Science 222, 1346 (1983)).

Also included are genes encoding resistance to: chloramphenicol (Herrera-Estrella et al., EMBO J. 2, 987 (1983)); methotrexate (Herrera-Estrella et al., Nature 303, 209 (1983); Meijer et al., Plant Mol. Biol. 16, 807 (1991)); hygromycin (Waldron et al., Plant Mol. Biol. 5, 103 (1985); Zhijian et al., Plant Science 108, 219 (1995); Meijer et al., Plant Mol. Bio. 16, 807 (1991)); streptomycin (Jones et al., Mol. Gen. Genet. 210, 86 (1987)); and spectinomycin (Bretagne-Sagnard et al., Transgenic Res. 5, 131 (1996)); bleomycin (Hille et al., Plant Mol. Biol. 7, 171 (1986)); sulfonamide (Guerineau et al., Plant Mol. Bio. 15, 127 (1990); bromoxynil (Stalker et al., Science 242, 419 (1988)); 2,4-D (Streber et al., Bio/Technology 7, 811 (1989)); phosphinothricin (DeBlock et al., EMBO J. 6, 2513 (1987)); spectinomycin (Bretagne-Sagnard and Chupeau, Transgenic Research 5, 131 (1996)).

Other selectable marker genes include the pat gene (for bialaphos and phosphinothricin resistance), the ALS gene for imidazolinone resistance, the HPH or HYG gene for hygromycin resistance, the Hm1 gene for resistance to the Hc-toxin, and other selective agents used routinely and known to one of ordinary skill in the art. See generally, Yarranton, Curr. Opin. Biotech. 3, 506 (1992); Chistopherson et al., Proc. Natl. Acad. Sci. USA 89, 6314 (1992); Yao et al., Cell 71, 63 (1992); Reznikoff, Mol. Microbiol. 6, 2419 (1992); BARKLEY ET AL., THE OPERON 177-220 (1980); Hu et al., Cell 48, 555 (1987); Brown et al., Cell 49, 603 (1987); Figge et al., Cell 52, 713 (1988); Deuschle et al., Proc. Natl. Acad. Sci. USA 86, 5400 (1989); Fuerst et al., Proc. Natl. Acad. Sci. USA 86, 2549 (1989); Deuschle et al., Science 248, 480 (1990); Labow et al., Mol. Cell. Biol. 10, 3343 (1990); Zambretti et al., Proc. Natl. Acad. Sci. USA 89, 3952 (1992); Baim et al., Proc. Natl. Acad. Sci. USA 88, 5072 (1991); Wyborski et al., Nuc. Acids Res, 19, 4647 (1991); Hillenand-Wissman, Topics in Mol. And Struc. Biol. 10, 143 (1989); Degenkolb et al., Antimicrob. Agents Chemother. 35, 1591 (1991); Kleinschnidt et al., Biochemistry 27, 1094 (1988); Gatz et al., Plant J. 2, 397 (1992); Gossen et al., Proc. Natl. Acad. Sci. USA 89, 5547 (1992); Oliva et al., Antimicrob. Agents Chemother. 36, 913 (1992); HLAVKA ET AL., HANDBOOK OF EXPERIMENTAL PHARMACOLOGY 78 (1985); and Gill et al., Nature 334, 721 (1988).

The nucleotide sequence of interest can additionally be operably linked to a sequence that encodes a transit peptide that directs expression of an encoded polypeptide of interest to a particular cellular compartment. Transit peptides that target protein accumulation in higher plant cells to the chloroplast, mitochondrion, vacuole, nucleus, and the endoplasmic reticulum (for secretion outside of the cell) are known in the art. Transit peptides that target proteins to the endoplasmic reticulum are desirable for correct processing of secreted proteins. Targeting protein expression to the chloroplast (for example, using the transit peptide from the RubP carboxylase small subunit gene) has been shown to result in the accumulation of very high concentrations of recombinant protein in this organelle. The pea RubP carboxylase small subunit transit peptide sequence has been used to express and target mammalian genes in plants (U.S. Pat. Nos. 5,717,084 and 5,728,925 to Herrera-Estrella et al.). Alternatively, mammalian transit peptides can be used to target recombinant protein expression, for example, to the mitochondrion and endoplasmic reticulum. It has been demonstrated that plant cells recognize mammalian transit peptides that target endoplasmic reticulum (U.S. Pat. Nos. 5,202,422 and 5,639,947 to Hiatt et al.).

Further, the expression cassette can comprise a 5′ leader sequence that acts to enhance expression (transcription, post-transcriptional processing and/or translation) of an operably associated nucleotide sequence of interest. Leader sequences are known in the art and include sequences from: picornavirus leaders, e.g., EMCV leader (Encephalomyocarditis 5′ noncoding region; Elroy-Stein et al., Proc. Natl. Acad. Sci USA, 86, 6126 (1989)).; potyvirus leaders, e.g., TEV leader (Tobacco Etch Virus; Allison et al., Virology, 154, 9 (1986)); human immunoglobulin heavy-chain binding protein (BiP; Macajak and Sarnow, Nature 353, 90 (1991)); untranslated leader from the coat protein mRNA of alfalfa mosaic virus (AMV RNA 4; Jobling and Gehrke, Nature 325, 622 (1987)); tobacco mosaic virus leader (TMV; Gallie, MOLECULAR BIOLOGY OF RNA, 237-56 (1989)); and maize chlorotic mottle virus leader (MCMV; Lommel et al., Virology 81, 382 (1991)). See also, Della-Cioppa et al., Plant Physiology 84, 965 (1987).

### IV. TRANSGENIC PLANTS, PLANT PARTS AND PLANT CELLS

The invention also provides transgenic plants, plant parts and plant cells produced by the methods of the invention and comprising the nucleic acids, expression cassettes and vectors described herein.

Accordingly, as one aspect the invention provides a cell comprising a nucleic acid, expression cassette, or vector as described herein. The cell can be transiently or stably transformed with the nucleic acid, expression cassette or vector. Further, the cell can be a cultured cell, a cell obtained from a plant, plant part, or plant tissue, or a cell in situ in a plant, plant part or plant tissue. Cells can be from any suitable species, including plant (e.g. rice), bacterial, yeast, insect and/or mammalian cells. In representative embodiments, the cell is a plant cell or bacterial cell.

The invention also provides a plant part (including a plant tissue culture) comprising a nucleic acid, expression cassette, or vector as described herein. The plant part can be transiently or stably transformed with the nucleic acid, expression cassette or vector. Further, the plant part can be in culture, can be a plant part obtained from a plant, or a plant part in situ. In representative embodiments, the plant part comprises a cell as described herein.

Seed comprising the nucleic acid, expression cassette, or vector as described herein are also provided. Optionally, the nucleic acid, expression cassette or vector is stably incorporated into the genome of the seed.

The invention also contemplates a transgenic plant comprising a nucleic acid, expression cassette, or vector as described herein. The plant can be transiently or stably transformed with a nucleic acid, expression cassette or vector. In representative embodiments, the plant comprises a cell or plant part as described herein.

Still further, the invention encompasses a crop comprising a plurality of the transgenic plants as described herein. Nonlimiting examples of the types of crops comprising a plurality of transgenic plants of the invention include an agricultural field, a golf course, a residential lawn or garden, a public lawn or garden, a road side planting, an orchard, and/or a recreational field (e.g., a cultivated area comprising a plurality of the transgenic plants of the invention).

Products harvested from the plants of the invention are also provided. Nonlimiting examples of a harvested product include a seed, a leaf, a stem, a shoot, a fruit, flower, root, biomass (e.g., for biofuel production) and/or extract.

In some embodiments, a processed product produced from the harvested product is provided. Nonlimiting examples of a processed product include a polypeptide (e.g., a recombinant polypeptide), an extract, a medicinal product (e.g., artemicin as an antimalarial agent), a fiber or woven textile, a fragrance, dried fruit, a biofuel (e.g., ethanol), a tobacco product (e.g., cured tobacco, cigarettes, chewing tobacco, cigars, and the like), an oil (e.g., sunflower oil, corn oil, canola oil, and the like), a nut or seed butter, a flour or meal (e.g., wheat or rice flour, corn meal) and/or any other animal feed (e.g., soy, maize, barley, rice, alfalfa) and/or human food product (e.g., a processed wheat, maize, rice or soy food product).

### V. METHODS OF INTRODUCING NUCLEIC ACIDS

The invention also provides methods of introducing a nucleic acid, expression cassette or vector as described herein to a target plant or plant cell (including callus cells or protoplasts), plant part, seed, plant tissue (including callus), and the like. The invention further comprises host plants, cells, plant parts, seed or tissue culture (including callus) transiently or stably transformed with the nucleic acids, expression cassettes or vectors as described herein.

The invention provides methods of introducing a GS1;2, GAD3, GAT1 and/or a MYB55 polypeptide into a plant material, e.g., a plant, plant part (including callus) or plant cell. In representative embodiments, the method comprises transforming a plant cell with a nucleic acid, expression cassette, or vector of the invention encoding the GS1;2, GAD3, GAT1 and/or MYB55 polypeptide to produce a transformed plant cell, and regenerating a stably transformed transgenic plant from the transformed plant cell.

The invention further encompasses transgenic plants (and progeny thereof), plant parts, and plant cells produced by the methods of the invention.

Also provided by the invention are seed produced from the inventive transgenic plants. Optionally, the seed comprise an isolated nucleic acid, expression cassette or vector as described herein stably incorporated into the genome.

Methods of introducing nucleic acids, transiently or stably, into plants, plant tissues, cells, protoplasts, seed, callus and the like are known in the art. Stably transformed nucleic acids can be incorporated into the genome. Exemplary transformation methods include biological methods using viruses and bacteria (e.g., Agrobacterium), physicochemical methods such as electroporation, floral dip methods, ballistic bombardment, microinjection, and the like. Other transformation technology includes the whiskers technology that is based on mineral fibers (see e.g., U.S. Pat. Nos. 5,302,523 and 5,464,765) and pollen tube transformation.

Other exemplary transformation methods include, without limitation, calcium-phosphate-mediated transformation, cyclodextrin-mediated transformation, nanoparticle-mediated transformation, sonication, infiltration, PEG-mediated nucleic acid uptake, as well as any other electrical, chemical, physical (mechanical) and/or biological mechanism that results in the introduction of nucleic acid into the plant cell, including any combination thereof. General guides to various plant transformation methods known in the art include Miki et al. (“Procedures for Introducing Foreign DNA into Plants” in Methods in Plant Molecular Biology and Biotechnology, Glick, B. R. and Thompson, J. E., Eds. (CRC Press, Inc., Boca Raton, 1993), pages 67-88) and Rakowoczy-Trojanowska (Cell. Mol. Biol. Lett. 7:849-858 (2002)).

Thus, in some particular embodiments, the method of introducing into a plant, plant part, plant tissue, plant cell, protoplast, seed, callus and the like comprises bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery, microinjection, sonication, infiltration, polyethyleneglycol-mediated transformation, any other electrical, chemical, physical and/or biological mechanism that results in the introduction of nucleic acid into the plant, plant part and/or cell thereof, or a combination thereof.

In one form of direct transformation, the vector is microinjected directly into plant cells by use of micropipettes to mechanically transfer the recombinant DNA (Crossway, Mol. Gen. Genetics 202: 179 (1985)).

In another protocol, the genetic material is transferred into the plant cell using polyethylene glycol (Krens, et al. Nature 296, 72 (1982)).

In still another method, protoplasts are fused with minicells, cells, lysosomes, or other fusible lipid-surfaced bodies that contain the nucleotide sequence to be transferred to the plant (Fraley, et al., Proc. Natl. Acad. Sci. USA 79, 1859 (1982)).

Nucleic acids may also be introduced into the plant cells by electroporation (Fromm et al., Proc. Natl. Acad. Sci. USA 82, 5824 (1985)). In this technique, plant protoplasts are electroporated in the presence of nucleic acids comprising the expression cassette. Electrical impulses of high field strength reversibly permeabilize biomembranes allowing the introduction of the nucleic acid. Electroporated plant protoplasts reform the cell wall, divide and regenerate. One advantage of electroporation is that large pieces of DNA, including artificial chromosomes, can be transformed by this method.

Ballistic transformation typically comprises the steps of: (a) providing a plant material as a target; (b) propelling a microprojectile carrying the heterologous nucleotide sequence at the plant target at a velocity sufficient to pierce the walls of the cells within the target and to deposit the nucleotide sequence within a cell of the target to thereby provide a transformed target. The method can further include the step of culturing the transformed target with a selection agent and, optionally, regeneration of a transformed plant. As noted below, the technique may be carried out with the nucleotide sequence as a precipitate (wet or freeze-dried) alone, in place of the aqueous solution containing the nucleotide sequence.

Any ballistic cell transformation apparatus can be used in practicing the present invention. Exemplary apparatus are disclosed by Sandford et al. (Particulate Science and Technology 5, 27 (1988)), Klein et al. (Nature 327, 70 (1987)), and in EP 0 270 356. Such apparatus have been used to transform maize cells (Klein et al., Proc. Natl. Acad. Sci. USA 85, 4305 (1988)), soybean callus (Christou et al., Plant Physiol. 87, 671 (1988)), McCabe et al., BioTechnology 6, 923 (1988), yeast mitochondria (Johnston et al., Science 240, 1538 (1988)), and Chlamydomonas chloroplasts (Boynton et al., Science 240, 1534 (1988)).

Alternately, an apparatus configured as described by Klein et al. (Nature 70, 327 (1987)) may be utilized. This apparatus comprises a bombardment chamber, which is divided into two separate compartments by an adjustable-height stopping plate. An acceleration tube is mounted on top of the bombardment chamber. A macroprojectile is propelled down the acceleration tube at the stopping plate by a gunpowder charge. The stopping plate has a borehole formed therein, which is smaller in diameter than the microprojectile. The macroprojectile carries the microprojectile(s), and the macroprojectile is aimed and fired at the borehole. When the macroprojectile is stopped by the stopping plate, the microprojectile(s) is propelled through the borehole. The target is positioned in the bombardment chamber so that a microprojectile(s) propelled through the bore hole penetrates the cell walls of the cells in the target and deposit the nucleotide sequence of interest carried thereon in the cells of the target. The bombardment chamber is partially evacuated prior to use to prevent atmospheric drag from unduly slowing the microprojectiles. The chamber is only partially evacuated so that the target tissue is not desiccated during bombardment. A vacuum of between about 400 to about 800 millimeters of mercury is suitable.

In alternate embodiments, ballistic transformation is achieved without use of microprojectiles. For example, an aqueous solution containing the nucleotide sequence of interest as a precipitate may be carried by the macroprojectile (e.g., by placing the aqueous solution directly on the plate-contact end of the macroprojectile without a microprojectile, where it is held by surface tension), and the solution alone propelled at the plant tissue target (e.g., by propelling the macroprojectile down the acceleration tube in the same manner as described above). Other approaches include placing the nucleic acid precipitate itself (“wet” precipitate) or a freeze-dried nucleotide precipitate directly on the plate-contact end of the macroprojectile without a microprojectile. In the absence of a microprojectile, it is believed that the nucleotide sequence must either be propelled at the tissue target at a greater velocity than that needed if carried by a microprojectile, or the nucleotide sequenced caused to travel a shorter distance to the target (or both).

It particular embodiments, the nucleotide sequence is delivered by a microprojectile. The microprojectile can be formed from any material having sufficient density and cohesiveness to be propelled through the cell wall, given the particle's velocity and the distance the particle must travel. Non-limiting examples of materials for making microprojectiles include metal, glass, silica, ice, polyethylene, polypropylene, polycarbonate, and carbon compounds (e.g., graphite, diamond). Non-limiting examples of suitable metals include tungsten, gold, and iridium. The particles should be of a size sufficiently small to avoid excessive disruption of the cells they contact in the target tissue, and sufficiently large to provide the inertia required to penetrate to the cell of interest in the target tissue. Particles ranging in diameter from about one-half micrometer to about three micrometers are suitable. Particles need not be spherical, as surface irregularities on the particles may enhance their carrying capacity.

The nucleotide sequence may be immobilized on the particle by precipitation. The precise precipitation parameters employed will vary depending upon factors such as the particle acceleration procedure employed, as is known in the art. The carrier particles may optionally be coated with an encapsulating agents such as polylysine to improve the stability of nucleotide sequences immobilized thereon, as discussed in EP 0 270 356 (column 8).

Alternatively, plants may be transformed using Agrobacterium tumefaciens or Agrobacterium rhizogenes. Agrobacterium-mediated nucleic acid transfer exploits the natural ability of A. tumefaciens and A. rhizogenes to transfer DNA into plant chromosomes. Agrobacterium is a plant pathogen that transfers a set of genes encoded in a region called T-DNA of the Ti and Ri plasmids of A. tumefaciens and A. rhizogenes, respectively, into plant cells. The typical result of transfer of the Ti plasmid is a tumorous growth called a crown gall in which the T-DNA is stably integrated into a host chromosome. Integration of the Ri plasmid into the host chromosomal DNA results in a condition known as “hairy root disease”. The ability to cause disease in the host plant can be removed by deletion of the genes in the T-DNA without loss of DNA transfer and integration. The DNA to be transferred is attached to border sequences that define the end points of an integrated T-DNA.

Transfer by means of engineered Agrobacterium strains has become routine for many dicotyledonous plants. Some difficulty has been experienced, however, in using Agrobacterium to transform monocotyledonous plants, in particular, cereal plants. However, Agrobacterium mediated transformation has been achieved in several monocot species, including cereal species such as rye, maize (Rhodes et al., Science 240, 204 (1988)), and rice (Hiei et al., (1994) Plant J. 6:271).

While the following discussion will focus on using A. tumefaciens to achieve gene transfer in plants, those skilled in the art will appreciate that this discussion also applies to A. rhizogenes. Transformation using A. rhizogenes has developed analogously to that of A. tumefaciens and has been successfully utilized to transform, for example, alfalfa, Solanum nigrum L., and poplar (U.S. Pat. No. 5,777,200 to Ryals et al.). As described by U.S. Pat. No. 5,773,693 to Burgess et al., it is preferable to use a disarmed A. tumefaciens strain (as described below), however, the wild-type A. rhizogenes may be employed. An illustrative strain of A. rhizogenes is strain 15834.

In particular protocols, the Agrobacterium strain is modified to contain the nucleotide sequences to be transferred to the plant. The nucleotide sequence to be transferred is incorporated into the T-region and is typically flanked by at least one T-DNA border sequence, optionally two T-DNA border sequences. A variety of Agrobacterium strains are known in the art particularly, and can be used in the methods of the invention. See, e.g., Hooykaas, Plant Mol. Biol. 13, 327 (1989); Smith et al., Crop Science 35, 301 (1995); Chilton, Proc. Natl. Acad. Sci. USA 90, 3119 (1993); Mollony et al., Monograph Theor. Appl. Genet NY 19, 148 (1993); Ishida et al., Nature Biotechnol. 14, 745 (1996); and Komari et al., The Plant Journal 10, 165 (1996).

In addition to the T-region, the Ti (or Ri) plasmid contains a vir region. The vir region is important for efficient transformation, and appears to be species-specific.

Two exemplary classes of recombinant Ti and Ri plasmid vector systems are commonly used in the art. In one class, called “cointegrate,” the shuttle vector containing the gene of interest is inserted by genetic recombination into a non-oncogenic Ti plasmid that contains both the cis-acting and trans-acting elements required for plant transformation as, for example, in the PMLJ1 shuttle vector of DeBlock et al., EMBO J 3, 1681 (1984), and the non-oncogenic Ti plasmid pGV2850 described by Zambryski et al., EMBOJ 2, 2143 (1983). In the second class or “binary” system, the gene of interest is inserted into a shuttle vector containing the cis-acting elements required for plant transformation. The other necessary functions are provided in trans by the non-oncogenic Ti plasmid as exemplified by the pBIN19 shuttle vector described by Bevan, Nucleic Acids Research 12, 8711 (1984), and the non-oncogenic Ti plasmid PAL4404 described by Hoekma, et al., Nature 303, 179 (1983).

Binary vector systems have been developed where the manipulated disarmed T-DNA carrying the heterologous nucleotide sequence of interest and the vir functions are present on separate plasmids. In this manner, a modified T-DNA region comprising foreign DNA (the nucleic acid to be transferred) is constructed in a small plasmid that replicates in E. coli. This plasmid is transferred conjugatively in a tri-parental mating or via electroporation into A. tumefaciens that contains a compatible plasmid with virulence gene sequences. The vir functions are supplied in trans to transfer the T-DNA into the plant genome. Such binary vectors are useful in the practice of the present invention.

In particular embodiments of the invention, super-binary vectors are employed. See, e.g., U.S. Pat. No. 5,591,615 and EP 0 604 662. Such a super-binary vector has been constructed containing a DNA region originating from the hypervirulence region of the Ti plasmid pTiBo542 (Jin et al., J. Bacteriol. 169, 4417 (1987)) contained in a super-virulent A. tumefaciens A281 exhibiting extremely high transformation efficiency (Hood et al., Biotechnol. 2, 702 (1984); Hood et al., J. Bacteriol. 168, 1283 (1986); Komari et al., J. Bacteriol. 166, 88 (1986); Jin et al., J. Bacteriol. 169, 4417 (1987); Komari, Plant Science 60, 223 (1987); ATCC Accession No. 37394.

Exemplary super-binary vectors known to those skilled in the art include pTOK162 (Japanese patent Appl. (Kokai) No. 4-222527, EP 504,869, EP 604,662, and U.S. Pat. No. 5,591,616) and pTOK233 (Komari, Plant Cell Reports 9, 303 (1990); Ishida et al., Nature Biotechnology 14, 745 (1996)). Other super-binary vectors may be constructed by the methods set forth in the above references. Super-binary vector pTOK162 is capable of replication in both E. coli and in A. tumefaciens. Additionally, the vector contains the virB, virC and virG genes from the virulence region of pTiBo542. The plasmid also contains an antibiotic resistance gene, a selectable marker gene, and the nucleic acid of interest to be transformed into the plant. The nucleic acid to be inserted into the plant genome is typically located between the two border sequences of the T region. Super-binary vectors of the invention can be constructed having the features described above for pTOK162. The T-region of the super-binary vectors and other vectors for use in the invention are constructed to have restriction sites for the insertion of the genes to be delivered. Alternatively, the DNA to be transformed can be inserted in the T-DNA region of the vector by utilizing in vivo homologous recombination. See, Herrera-Esterella et al., EMBO J. 2, 987 (1983); Horch et al., Science 223, 496 (1984). Such homologous recombination relies on the fact that the super-binary vector has a region homologous with a region of pBR322 or other similar plasmids. Thus, when the two plasmids are brought together, a desired gene is inserted into the super-binary vector by genetic recombination via the homologous regions.

In plants stably transformed by Agrobacteria-mediated transformation, the nucleotide sequence of interest is incorporated into the plant nuclear genome, typically flanked by at least one T-DNA border sequence and generally two T-DNA border sequences.

Plant cells may be transformed with Agrobacteria by any means known in the art, e.g., by co-cultivation with cultured isolated protoplasts, or transformation of intact cells or tissues. The first uses an established culture system that allows for culturing protoplasts and subsequent plant regeneration from cultured protoplasts. Identification of transformed cells or plants is generally accomplished by including a selectable marker in the transforming vector, or by obtaining evidence of successful bacterial infection.

Methods of introducing a nucleic acid into a plant can also comprise in vivo modification of genetic material, methods for which are known in the art. For example, in vivo modification can be used to insert an isolated nucleic acid as described herein into the plant genome.

Suitable methods for in vivo modification include the techniques described in Gao et. al., Plant J. 61, 176 (2010); Li et al., Nucleic Acids Res. 39, 359 (2011); U.S. Pat. Nos. 7,897,372 and 8,021,867; U.S. Patent Publication No. 2011/0145940 and in International Patent Publication Nos. WO 2009/114321, WO 2009/134714 and WO 2010/079430. For example, one or more transcription affector-like nucleases (TALEN) and/or one or more meganucleases may be used to incorporate an isolated nucleic acid as described herein into the plant genome. In representative embodiments, the method comprises cleaving the plant genome at a target site with a TALEN and/or a meganuclease and providing a polynucleotide that comprises a sequence that is homologous to at least a portion of the target site and further comprises an isolated nucleic acid of the invention, such that homologous recombination occurs and results in the insertion of the isolated nucleic acid into the genome.

Protoplasts, which have been transformed by any method known in the art, can also be regenerated to produce intact plants using known techniques.

Plant regeneration from cultured protoplasts is described in Evans et al., Handbook of Plant Cell Cultures, Vol. 1: (MacMilan Publishing Co. New York, 1983); and Vasil I. R. (ed.), Cell Culture and Somatic Cell Genetics of Plants, Acad. Press, Orlando, Vol. I, 1984, and Vol. II, 1986). Essentially all plant species can be regenerated from cultured cells or tissues, including but not limited to, all major species of sugar-cane, sugar beet, cotton, fruit trees, and legumes.

Means for regeneration vary from species to species of plants, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It is also advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

Alternatively, transgenic plants may be produced using the floral dip method (See, e.g., Clough and Bent (1998) Plant Journal 16:735-743, which avoids the need for plant tissue culture or regeneration. In one representative protocol, plants are grown in soil until the primary inflorescence is about 10 cm tall. The primary inflorescence is cut to induce the emergence of multiple secondary inflorescences. The inflorescences of these plants are typically dipped in a suspension of Agrobacterium containing the vector of interest, a simple sugar (e.g., sucrose) and surfactant. After the dipping process, the plants are grown to maturity and the seeds are harvested. Transgenic seeds from these treated plants can be selected by germination under selective pressure (e.g., using the chemical bialaphos). Transgenic plants containing the selectable marker survive treatment and can be transplanted to individual pots for subsequent analysis. See Bechtold, N. and Pelletier, G. Methods Mol Biol 82, 259-266 (1998); Chung, M. H. et al. Transgenic Res 9, 471-476 (2000); Clough, S. J. and Bent, A. F. Plant J 16, 735-743 (1998); Mysore, K. S. et al. Plant J 21, 9-16 (2000); Tague, B.W. Transgenic Res 10, 259-267 (2001); Wang, W. C. et al. Plant Cell Rep 22, 274-281 (2003); Ye, Q.N. et al. Plant J., 19:249-257 (1999).

The particular conditions for transformation, selection and regeneration can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine what is an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

The present invention is more particularly described in the following examples that are intended as illustrative only since numerous modifications and variations therein will be apparent to those skilled in the art.

### EXAMPLES

As used in the following Examples, the term “normal growth conditions” refers to growth conditions comprising 29° C. daytime temperatures, 23° C. nighttime temperatures, 12 hours of light (approximately 500 μmol m⁻² s⁻¹) during the daytime and 12 hours of dark during the nighttime.

As used in the following Examples, the term “normal temperature conditions” refers to growth conditions comprising 29° C. daytime temperatures and 23° C. nighttime temperatures.

As used in the following Examples, the term “high temperature conditions” refers to growth conditions comprising 35° C. daytime temperatures and 26° C. nighttime temperatures.

As used in the following Examples, the term “normal daylight conditions” refers to growth conditions comprising 12 hours of light (approximately 500 μmol m⁻² s⁻¹) during the daytime and 12 hours of dark during the nighttime.

As used in the following Examples, the term “long daylight conditions” refers to growth conditions comprising 16 hours of light (approximately 500 μmol m⁻² s⁻¹) during the daytime and 8 hours of dark during the nighttime.

### Example 1

### Characterization of OsMYB55

The 867 bp full length cDNA sequence (SEQ ID NO:4; FIG. 1E) of OsMYB55 encodes an R2R3-MYB transcription factor predicted to be 289 amino acids in length (SEQ ID NO:5; FIG. 1F). A BLAST® (National Center for Biotechnology Information, Bethesda, Md.) search was used to identify homologues of OsMYB55. Amino acid sequences of the closest homologs (SEQ ID NOs:6-13; FIGS. 17A-H) were used to generate a phylogenetic tree showing the similarity between OsMYB55 and its homologues (FIG. 1A).

The genomic DNA sequence containing the 5′UTR, promoter sequence, the MYB55 coding region (containing three exons and two introns) and 3′ UTR are shown in FIG. 1D (SEQ ID NO:3); the 5′ UTR and promoter sequence alone are shown in FIG. 1C (SEQ ID NO:2). A 2134 bp portion of the OsMYB55 promoter sequence (lacking nucleotides −1 to −5) is shown in FIG. 1B (SEQ ID NO:1) and was used to construct a GUS reporter construct (Example 3).

To understand the regulation of the OsMYB55 gene, an in-silico analysis of the OsMYB55 promoter region (2100 bp) was carried out using the PlantCARE database (Flanders Interuniversity Institute for Biotechnology, Zwijnaarde, Belgium; available at http://bioinformatics.psb.ugent.be/webtools/plantcare/html/). Numerous potential CAREs and TFBS were identified in the OsMYB55 promoter region (FIGS. 2A-2B).

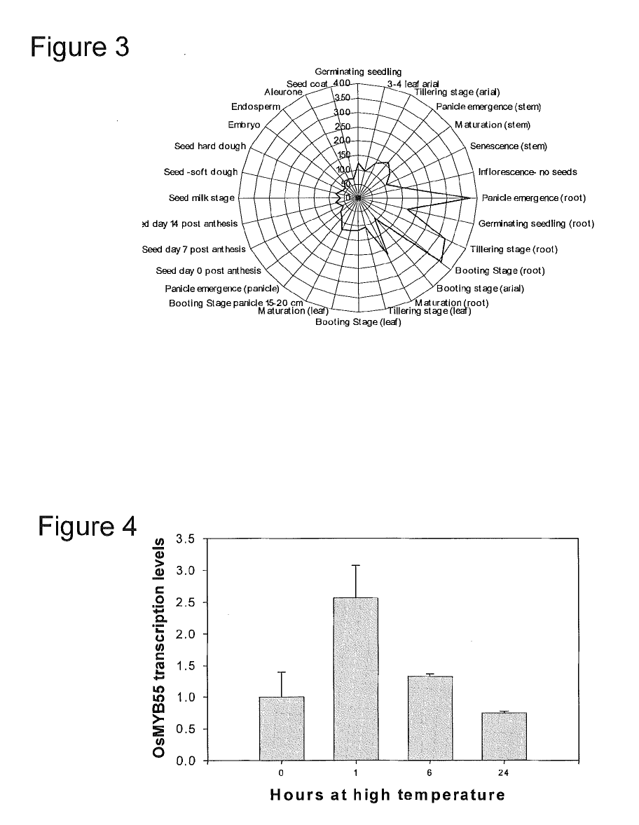

Global expression analysis revealed that OsMYB55 is differentially expressed in plant tissues and that its expression varies throughout the plant's life cycle (FIG. 3). Transcript levels were higher during vegetative stages up to tillering and the inflorescence stage. Transcription was higher in root tissues than in leaf tissues during those stages. OsMYB55's lowest expression level was observed in seeds, both during seed development at seed maturation.

### Example 2

### OsMYB55 Expression is Up-Regulated in Response to Heat Stress: OsMYB55 Transcripts

Seeds from wild-type rice plants were planted in 500 ml pots containing a growth media comprising peat moss and vermiculite in a ratio of 1:4. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions for four weeks.

Following four weeks of growth under normal growth conditions, plants were exposed to 45° C. for 0, 1, 6 or 24 hours. Leaves were harvested from each plant, frozen immediately in liquid nitrogen and stored at −80° C.

Quantitative real-time RT-PCR analysis of the leaves showed that OsMYB55 expression was up-regulated following exposure to 45° C. for one hour and that OsMYB55 expression returned to basal levels following exposure to 45° C. for 6 or 24 hours (FIG. 4).

### Example 3

### OsMYB55 Expression is Up-Regulated in Response to Heat Stress: GUS Reporter Protein

To generate the OsMYB55promoter-GUS construct, a 2134 base pair fragment of the OsMYB55 promoter region was amplified from genomic DNA using the OsMYB55promoter-BamH1 forward primer (5′-TGGTGAGGAGGATTGTGCAAGGATCCGCG-3′; SEQ ID NO:21) and the OsMYB55promoter-EcoR1 reverse primer (5′-CCGGAATTCTTGCACAATCCTCCT CACCA-3′; SEQ ID NO:22).

DNA was isolated from four-week-old plants grown under normal growth conditions using the cetyl trimethylammonium bromide (CTAB) extraction method. The amplified fragment (SEQ ID NO:1; FIG. 1B) was cloned into the multiple cloning site of the pCAMBIA1391Z (Cambia, Brisbane, Australia) between the BamHI and EcoRI restriction sites to drive expression of the GUS reporter protein. Transgenic rice lines comprising the OsMYB55promoter-GUS construct were generated using Agrobacterium-mediated transformation, and positively transformed lines were selected according to the methods of Miki et al., PLANT PHYSIOL. 138(4):1903 (2005).

Seeds from transgenic rice plants expressing the OsMYB55promoter-GUS construct were planted in 500 ml pots containing a growth media comprising peat moss and vermiculite in a ratio of 1:4. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions for four weeks.

Following four weeks of growth under normal growth conditions, plants were exposed to 29° C. or 45° C. for 0, 1, 6 or 24 hours. Plant tissues were harvested 24 hours after treatment and stained by immersion in 0.1 M sodium citrate-HCl buffer pH 7.0 containing 1 mg/ml 5-bromo-4-chloro-3-indolyl-β-D-glucuronic acid (X-Gluc) (Biosynth International, Inc., Itasca, Ill.), vacuum infiltration for five minutes and incubation at 37° C. for 16 hours. Chlorophyll was removed from the tissues by incubating the tissues in 75% ethanol. The samples were conserved in glycerol 10% until examination. Hand sections were prepared and investigated using a light microscopy.

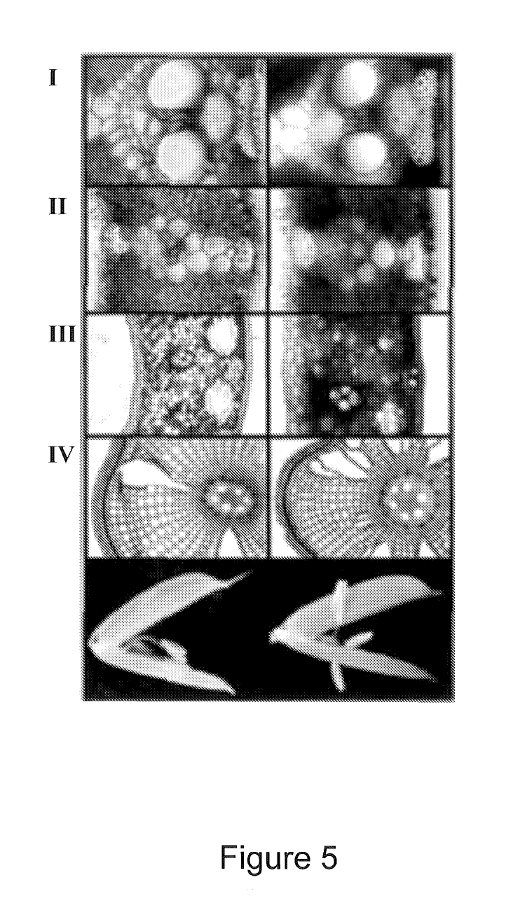

As shown in FIG. 5, GUS expression was higher in plants exposed to 45° C. for 24 hours than in plants exposed to 29° C. for 24 hours.

### Example 4

### Transgenic Rice Plants Overexpressing OsMYB55

Since OsMYB55 expression is up-regulated in response to high temperatures, we investigated whether OsMYB55 expression plays a role in one or more heat stress responses and/or heat tolerance. Experiments were carried out at different developmental stages to determine the effect of OsMYB55 overexpression on plant thermotolerance.

Constructs for over-expressing OsMYB55 were created using the maize ubiquitin promoter. Agrobacterium-mediated transformation was used to generate transgenic plants. Positively transformed plants were selected using the phosphomannose isomerase (PMI) test (Negrotto et al. PLANT CELL REP. 19:798 (2000)).

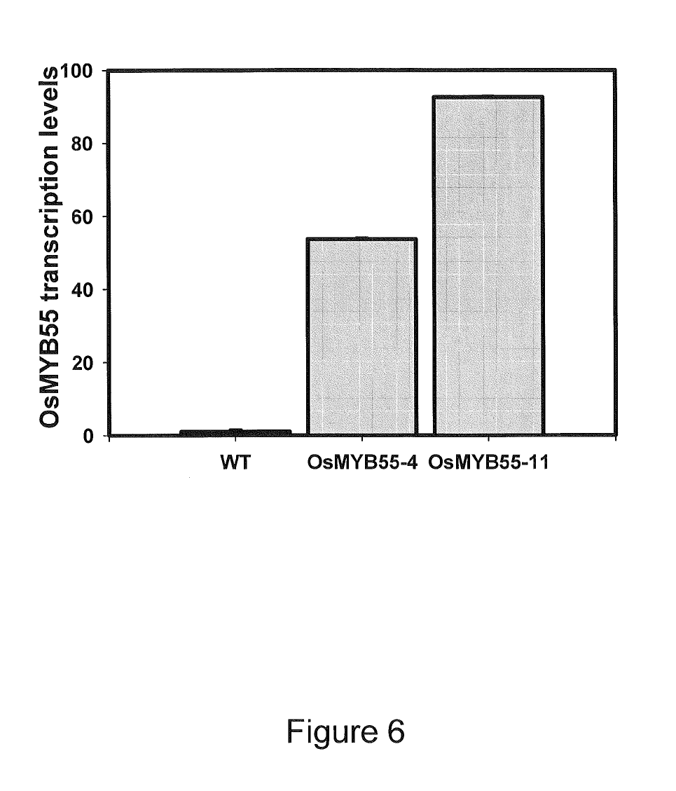

Expression analysis showed that OsMYB55 expression following four weeks of growth under normal growth conditions was fifty to ninety times higher in the leaves of transgenic rice plants overexpressing OsMYB55 as compared to wild-type rice plants (FIG. 6).

### Example 5

### OsMYB55 Overexpression Increases Coleoptile Length at High Temperatures

Seeds from wild-type rice plants and transgenic rice plants overexpressing OsMYB55 were germinated and grown for four days at 28° C. or 39° C.

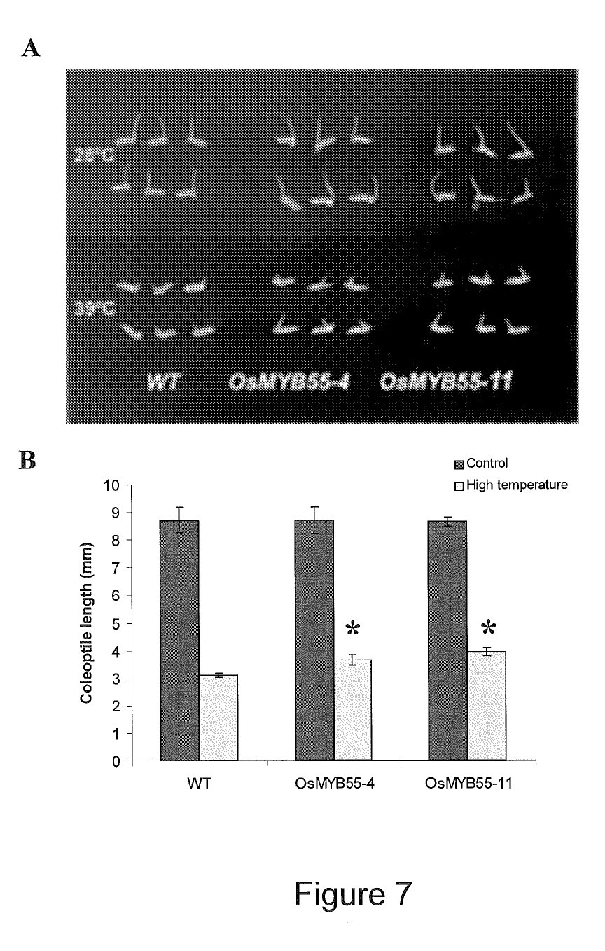

As shown in FIGS. 7A and 7B, although coleoptile length was reduced in all of the plants grown at 39° C., the coleoptiles of transgenic rice plants overexpressing OsMYB55 grown at 39° C. were significantly longer than those of their wild-type counterparts.

### Example 6

### OsMYB55 Overexpression Enhances Growth Under Long Daylight Conditions

Seeds from wild-type rice plants and transgenic rice plants overexpressing QsMYB55 were planted in 500 ml pots containing Turface® MVP® (PROFILE Products, LLC, Buffalo Grove, Ill.) (a 100% baked calcined clay growth media with grain size between 2.5 and 3.5 mm). Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for 4 weeks under long daylight conditions with either normal temperature conditions or high temperature conditions.

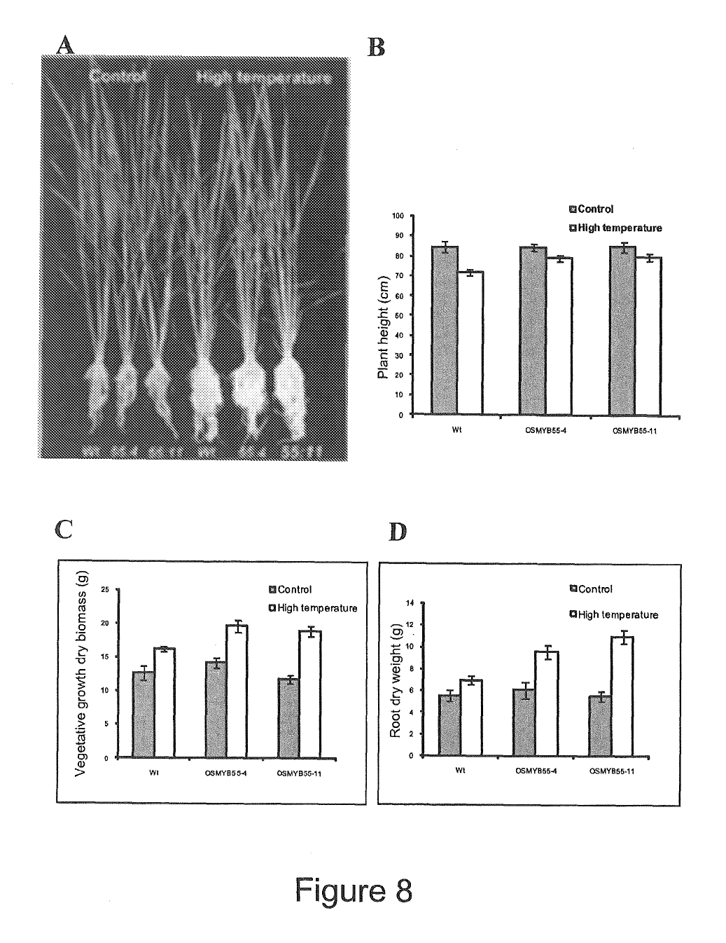

As shown in FIGS. 8A-8D, there were no significant differences in the heights, vegetative biomasses and root biomasses of plants grown under normal temperature conditions. Wild-type rice plants grown under high temperature conditions showed a decrease in plant height (FIG. 8B) and an increase in dry biomass (FIGS. 8C-8D). Transgenic rice plants overexpressing OsMYB55 grown under high temperature conditions showed less reduction in plant height (FIG. 8B) and a significant increase in plant biomass as compared to their wild-type counterparts (FIGS. 8C-8D).

### Example 7

### OsMYB55 Overexpression Enhances Growth under Normal Daylight Conditions

Seeds from wild-type rice plants and transgenic rice plants overexpressing OsMYB55 were planted in 500 ml pots containing a growth media comprising peat moss and vermiculite in a ratio of 1:4. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under normal daylight conditions with either normal temperature conditions or high temperature conditions.

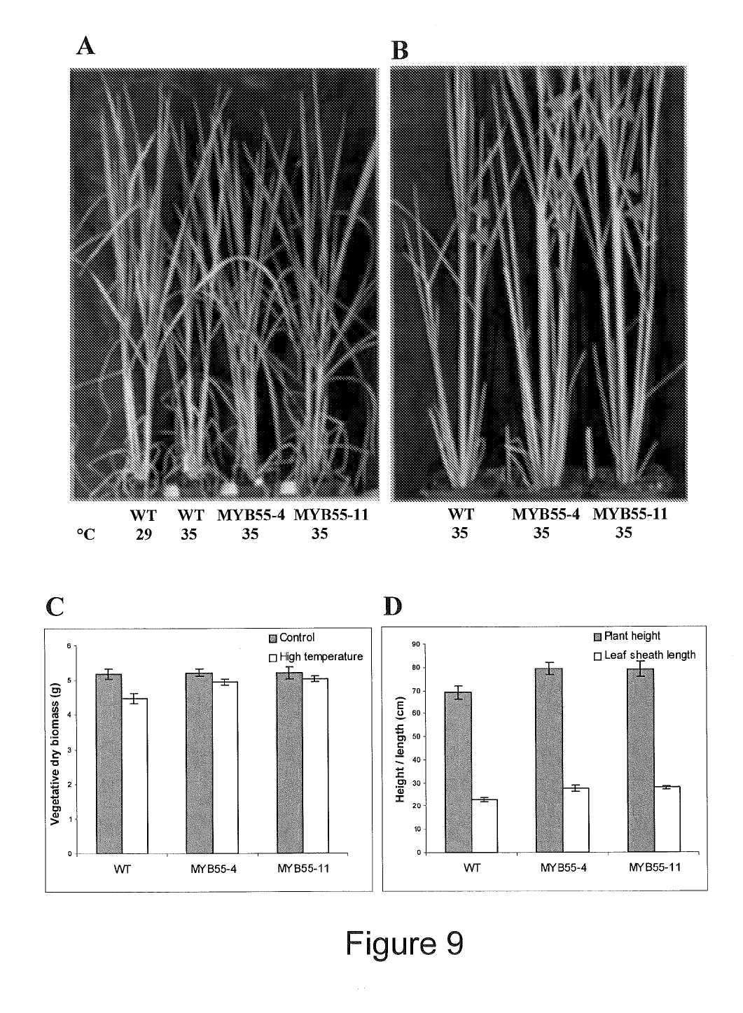

As shown in FIGS. 9A-9D, there were no significant differences in the heights and vegetative biomasses of plants grown under normal temperature conditions. Wild-type rice plants grown at high temperatures showed a decrease in plant height (FIG. 9C), vegetative biomass and leaf sheath length. Transgenic rice plants overexpressing OsMYB55 grown under high temperature conditions showed less reduction in vegetative biomass as compared to their wild-type counterparts (FIG. 9C). Overexpression of OsMYB55 also moderated the negative effects of the high temperature conditions on the height and leaf sheath length of transgenic rice plants overexpressing OsMYB55.

### Example 8

### The Deleterious Effects of Growth at a Continuous High Temperature are More Severe Under Long Daylight Conditions than Normal Daylight Conditions

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for 4, 9, 11 or 17 weeks under normal growth conditions, under normal daylight conditions with high temperature conditions or under long daylight conditions with either normal temperature conditions or high temperature conditions.

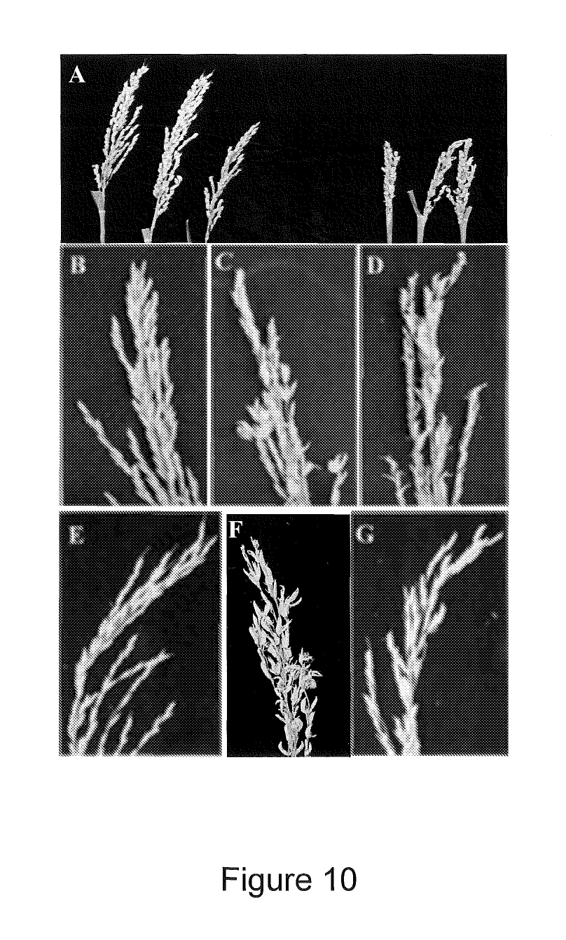

As shown in FIGS. 10A-10G, growth at a continuous high temperature caused deformations of the inflorescences and resulted in complete seed set failure as compared to growth under normal temperature conditions. Overexpression of OsMYB55 did not significantly reduce the occurrence of inflorescence deformations or seed set failure. For both wild-type rice plants and transgenic rice plants overexpressing OsMYB55, the deleterious effects of growth at a continuous high temperature were more severe in plants grown under long daylight conditions than in plants grown under neutral day conditions.

### Example 9

### OsMYB55 Overexpression Improves Plant to High Temperatures

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under normal growth conditions or under normal daylight conditions with high temperature conditions. Following the four-week treatment period, plants were grown under normal growth conditions until harvesting (about 12 weeks).

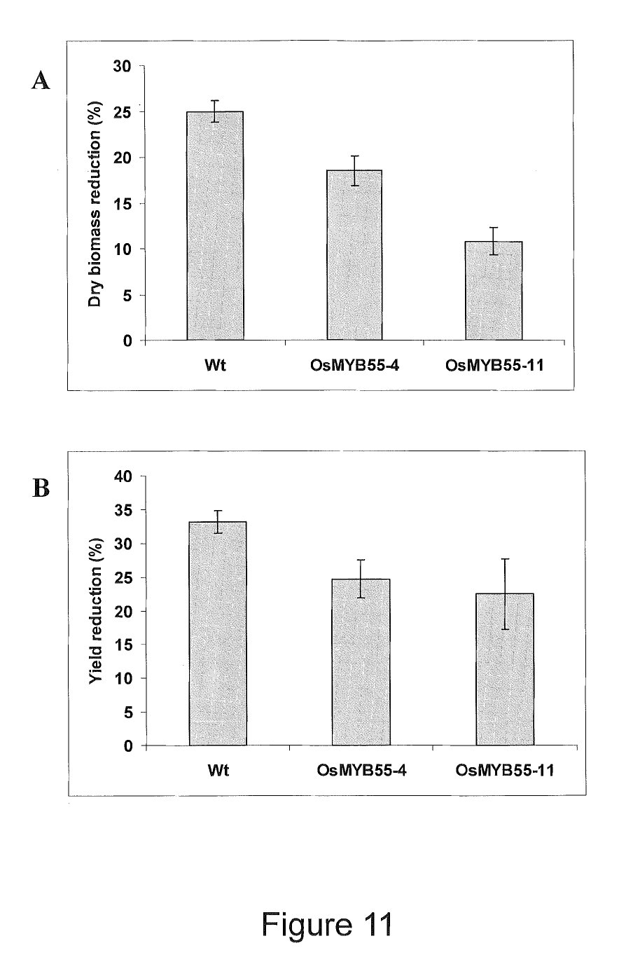

As shown in FIGS. 11A-11B, although growth under high temperature conditions for four weeks resulted in a significant reduction in the total dry biomasses and grain yields of both wild-type rice plants and transgenic rice plants overexpressing OsMYB55, those reductions were less pronounced in transgenic rice plants overexpressing OsMYB55.

### Example 10

### Rice Plants Expressing an OsMYB55-RNAi Construct

An OsMYB55-RNAi construct was prepared according to the methods of Miki et al., PLANT PHYSIOL. 138(4):1903 (2005). cDNA sequence fragments of OsMYB55 491 bp in length and with low similarity to other rice genes were amplified by PCR using the OsMYB55-491 forward primer (5′-CGTCAAGAACTACTGGAACAC C-3′; SEQ ID NO:23) and the OsMYB55-491 reverse primer (5′-CCATGTTCGGGAAGTA GCAC-3′; SEQ ID NO:24). The resultant fragment was cloned into the TOPO® pENTER cloning vector (Life Technologies Corp., Carlsbad, Calif.), and the inverted DNA sequences separated by a GUS intron sequence were generated by the site-specific recombination method in the pANDA binary vector described by Miki et al., PLANT PHYSIOL. 138(4)1903 (2005), downstream of the maize ubiquitin promoter using the Gateway LR Clonase Enzyme Mix (Life Technologies Corp., Carlsbad, Calif.). Transgenic rice lines were obtained using Agrobacterium-mediated transformation, and positively transformed lines were selected according to the methods of Miki et al., PLANT PHYSIOL. 138(4):1903 (2005).

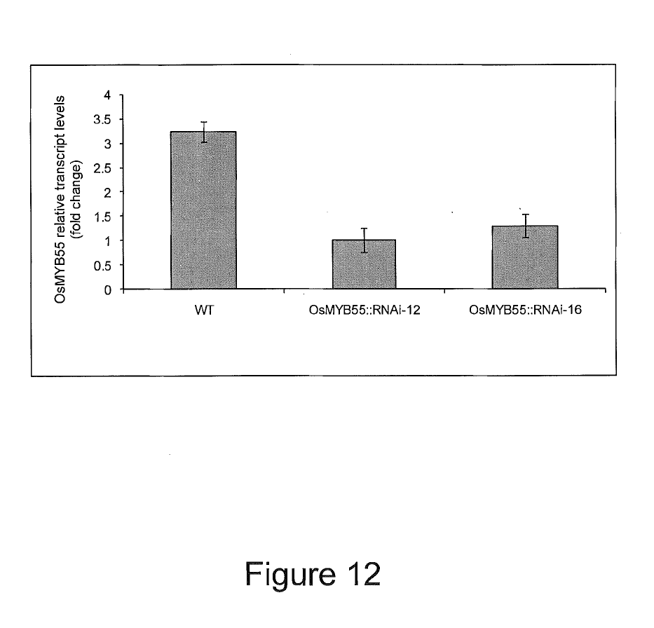

Although the transcript level of OsMYB55 was around three times less in four-week-old rice plants expressing the OsMYB55-RNAi construct (FIG. 12), no phenotypic difference was observed between wild-type rice plants and plants expressing the OsMYB55-RNAi construct. Without wishing to be bound by theory, it is currently believed that the lack of a discernible phenotype in rice plants expressing the OsMYB55-RNAi construct may be due to the large number of members of the R2R3-MYB transcription factor family and the high redundancy among this family.

### Example 11

### OsMYB55 Overexpression Enhances Total Leaf Amino Acid Content

To understand the physiological and molecular mechanisms underlying the enhancement of plant thermotolerance by OsMYB55, various plant tissues were collected and biochemical analyses were carried out to identify differences between wild type rice plants and transgenic rice plants overexpressing OsMYB55 (e.g., differences in sugar content, starch content, hydrogen peroxide content, amino acid content, etc.).

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under normal growth conditions or under normal daylight conditions with high temperature conditions.

Following the four-week treatment period, tissues were collected and freeze dried for 24 hours, then extracted three times using 0.75 mL of 100% methanol. Each extraction was carried out at 70° C. for 15 minutes. Extracts were subjected to chloroform purification by adding 500 μL extract to 355 μL of water and 835 μL chloroform. Following centrifugation, the upper phase was collected and freeze dried, then dissolved in deionized water. Total amino acid content was assayed according to the methods of Rosen, ARCH. BIOCHEM. BIOPHYS. 67:10 (1957).

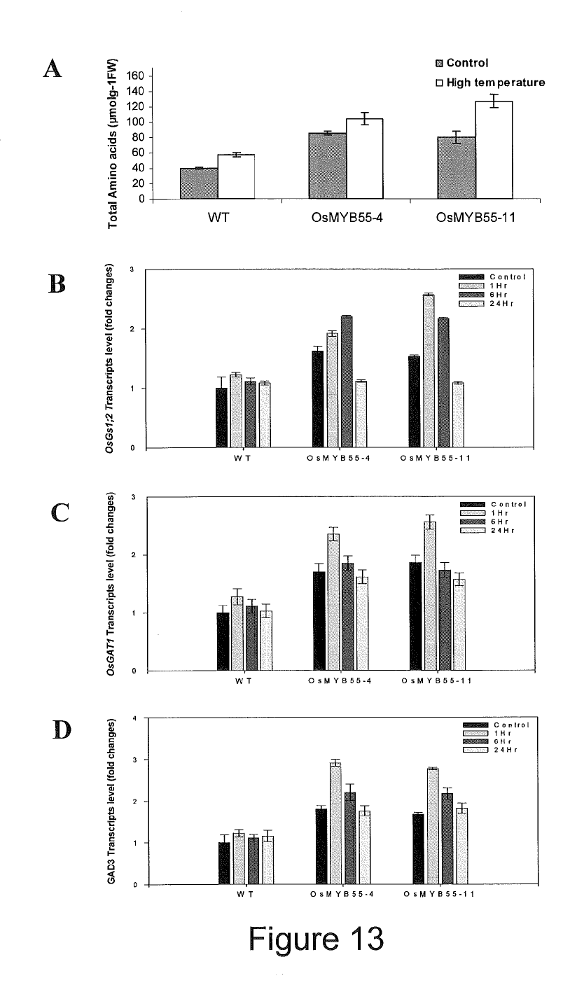

As shown in FIG. 13A, the leaves of transgenic rice plants overexpressing OsMYB55 had a higher total amino acid content than their wild-type counterparts when grown under long daylight conditions with either normal temperature conditions or high temperature conditions. Exposure to high temperature conditions increased the leaf amino acid contents of both wild-type rice plants and transgenic rice plants overexpressing OsMYB55. The leaf amino acid contents of transgenic rice plants overexpressing OsMYB55 were increased more significantly than were the leaf amino acid contents of wild-type rice plants.

### Example 12

### OsMYB55 Overexpression Up-Regulates the Expression of Genes Involved in Amino Acid Metabolism

Based on the total amino acids analysis results, we suspected that OsMYB55 might have a role in the activation of genes involved with amino acid metabolism. To test this hypothesis, a genome-wide transcriptome analysis was conducted using global microarray analysis of wild-type rice plants and transgenic rice plants overexpressing OcMYB55 exposed to high temperatures.

Because microarray analysis can miss more subtle changes in gene expression, quantitative real-time PCR was performed using primers corresponding to other genes involved in amino acids biosynthesis and transport. Quantitative real-time RT-PCR was carried out using specific primers designed from the sequence of the chosen genes. Total RNA was isolated from plant tissues using TRI-Reagent RNA isolation reagent (Sigma-Aldrich Corp., St. Louis, Mo.). To eliminate any residual genomic DNA, total RNA was treated with RQ1 RNase-free DNase (Promega Corp., Madison, Wis.). cDNA was synthesized from total RNA using the Reverse Transcription System kit (Quanta BioSciences, Inc., Gaithersburg, Md.). Primer Express 2.0 software (Applied Biosystems by Life Technologies Corp., Carlsbad, Calif.) was used to design the primers for the target genes. Relative quantification (RQ) values for each target gene relative to the internal control Actin2 were calculated using the 2CT method described by Livak and Schmittgen, METHODS 25:402 (2001).

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under long daylight conditions with normal temperature conditions for four weeks and then exposed to 45° C. for 0, 1, 6 or 24 hours.

Three candidate genes important for amino acid production were found to be up-regulated: OsGS1;2, OsGAT1 and OsGAD3 (FIGS. 13B-13D). GS1;2 is involved in converting glutamine into glutamic acid and represents one of the early steps in amino acids biosynthesis. GAT1, also known as carbamoyl phosphate synthetase, is involved in the first committed step in arginine biosynthesis in prokaryotes and eukaryotes (Holden et al., CURRENT OPIN. STRUCTURAL BIOL. 8:679 (1998)). The GAD genes are involved in converting the L-glutamic acid into GABA (Hiroshi, J. MOL. CATALYSIS B: ENZYMATIC 10:67 (2000)). Quantitative real-time PCR analysis revealed the up-regulation of the three aforementioned genes one hour after the exposure of rice plants to 45° C. The transcript levels of OsGAT1 and OsGAD3 decreased to nearly basal levels 6 hours after exposure to 45° C. (FIGS. 13C-13D), while OsGS1;2 transcript levels remained significantly increased 6 hours after exposure to 45° C. (FIG. 13B).

No significant difference was detected in the transcripts of others genes involved in amino acid metabolism.

### Example 13

### OsMYB55 Binds to Genes Involved in Amino Acid Metabolism

DNA sequences corresponding to the promoters of the genes identified in Example 12 (OsGS1;2, OsGAT1 and OsGAD3) were analyzed using the PlantCARE database (Flanders Interuniversity Institute for Biotechnology, Zwijnaarde, Belgium; available at http://bioinformatics.psb.ugent.be/webtools/plantcare/html/). The results showed that all three promoters contain a potential binding site for MYB proteins, a CAGTTA motif. The CAGTTA motif, located 1079 bp, 460 bp and 554 bp from the first ATG codon in the OsGS1;2, OsGAT1 and OsGAD3 cDNAs, respectively. Because the CAGTTA motif is a binding box for MYB transcription factors and is therefore a potential binding site for OsMYB55, electrophoretic mobility shift assays (EMSAs) were carried out to determine whether OsMYB55 binds to the CAGTTA-box of OsGS1;2, OsGAT1 or OsGAT3 in vitro.

Recombinant OsMYB55 was prepared as follows. The full-length coding region of OsMYB55 cDNA was amplified using the MYB55-P28-BamHI forward primer (5′-CGCGGAT CCATGGGGCGCGCGCCGT-3′; SEQ ID NO:25) and the MYB55-P28-Hinlll reverse primer (5′-CCCAAGCTTTGTCAGGGTGTTGCAGAGACCCTGT-3′; SEQ ID NO:26). The PCR product and a pET15b vector (Novagen®, EMB Biosciences) were digested with BamHI and HindIII. After ligation, the construct was transformed into Arctic Express (DE3) RIL competent cells (Agilent Technologies, Santa Clara, Calif.) according to the manufacturer's instructions. Recombinant OsMYB55 was purified using a His-tag purification system (Qiagen, Inc., Valencia, Calif.).

The potential OsMYB55 binding sites from the promoters of OsGS1;2, OsGAT1 and OsGAD3 were amplified using specific primers to produce DNA products containing one copy of their respective MYB binding boxes.

EMSAs were carried out with varying amounts of the recombinant OsMYB55 protein (0, 10, 20 or 40 pg) and the OsGS1;2, OsGAT1 and OsGAD3 DNA products (0 or 200 ng) using an EMSA kit (Cat. # E33075, Molecular Probes, Inc., Eugene, Oreg.). The DNA- and/or protein-containing samples were loaded into a Ready Gel TBE, gradient 4-20% polyacrylamide native gel (Bio-Rad Laboratories, Hercules, Calif.) at 200 V for 45 minutes. The DNA in the gel was stained using the SYBR® Green provided in the EMSA kit and visualized using the ChemiDoc™ imaging system (Bio-Rad Laboratories, Hercules, Calif.).

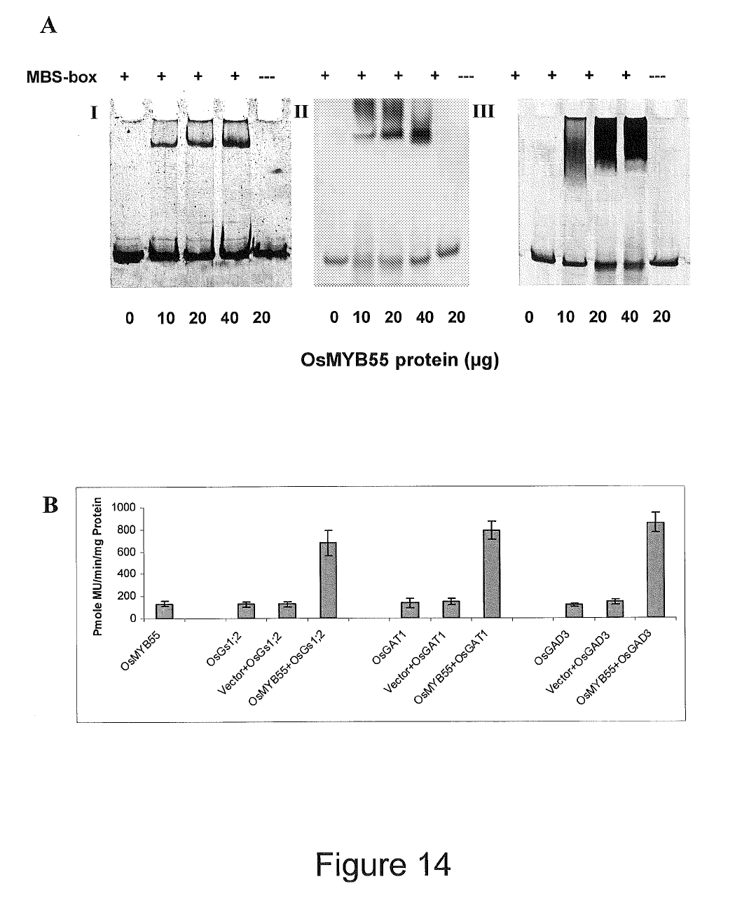

As shown in FIG. 14A, OsMYB55 strongly binds to the OsGS1;2, OsGAT1 and OsGAD3 promoter sequences containing the CAGTTA box motif.

### Example 14

### OsMYB55 Activates Genes Involved in Amino Acid Metabolism

Binding of the OsMYB55 protein to the promoter sequences of OsGs1;2, OsGAT1 and OsGAD3 supports the idea that OsMYB55 might enhance amino acid content through the activation of these genes. To investigate this hypothesis, a transcription activation assay using a transient gene expression strategy was carried out using GUS as a reporter protein.

DNA sequences corresponding to the OsGS1;2, OsGAD3 and OsGAT1 promoters were cloned into an intron-containing GUS reporter vector. The DNA sequence of the OsGS1;2, OsGAD3 and OsGAT1 promoters (1.5-2 kb upstream of the ATG start codon of the cDNA) was amplified from rice genomic DNA using the OsGS1;2 promoter forward primer (5′-CACCTGCGGTGAATGGAAGACGTTTG-3′; SEQ ID NO:27) and the OsGS1;2 promoter reverse primer (5′-TGCTCAAAGCAGAAGAGATCTGAATGAG-3′; SEQ ID NO:28), the OsGAT1 promoter forward primer (5′-CACCGACGGAGGAAGTAGTG TGGAACCAT-3′; SEQ ID NO:29) and the OsGAT1 promoter reverse primer (5′-TGGTGGTAGGGTG CGGC-3′; SEQ ID NO:30) or the OsGAD3 promoter forward primer (5′-CACCCAGATCAAATGTCA AAAGGGGCG-3′; SEQ ID NO:31) and the OsGAD3 promoter reverse primer (5′-CTTGCCT GCCGAGCTATCAACC-3′; SEQ ID NO:32). The resulting fragments were cloned into the TOPO pENTER vector (Life Technologies Corp., Carlsbad, Calif.), and the final construct was prepared by the site-specific recombination method in the DMC162 gateway vector by Gateway® LR Clonase® enzyme mix (Life Technologies Corp., Carlsbad, Calif.). OsMYB55 was inserted next to the 35S promoter in the DMC32 vector using the OsMYB55-Pent forward primer (5′-ATGGGGCGCGCGCCGTG-3′; SEQ ID NO:33) and the OsMYB55-Pent reverse primer (5′-CTATGTCAGGGTGTTGCAGAG ACC-3′; SEQ ID NO:34). This plasmid was used as an activator in the co-transformation transient expression analysis. To normalize the GUS activity values, the firefly (Photinus pyralis) luciferase gene driven by the 35S promoter in the pJD312 plasmid (kindly donated from Dr. Virginia Walbot, Stanford University) was used. Equal amounts of DNA from the different plasmid constructs were transformed by particle bombardment into four-week-old old tobacco (Nicotiana plumbaginifolia) leaves. After incubation for 48 hours at room temperature in the dark, total protein was extracted from each sample and GUS and luciferase activities were measured. GUS activity was determined by measuring the cleavage of β-glucuronidase substrate 4-methylumbelliferyl β-D-glucuronide (MUG). Luciferase activity was measured using the Luciferase Assay System kit (Cat. # E1500, Promega Corp., Madison, Wis.) following the manufacturers' instructions. Empty vectors were used as negative controls in this experiment.

As shown in FIG. 14B, OsMYB55 activated the expression of OsGs1;2, GAT1 and GADS in tobacco epidermal cells by almost eight-fold compared to the control experiment. In conjunction with the results of Example 13, these results indicate that OsMYB55 directly regulates the expression of OsGs1;2, OsGAT1 and OsGAD3.

### Example 15

### OsMYB55 Overexpression Enhances Leaf Glutamic Acid and Arginine Content

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under long daylight conditions with either normal temperatures conditions or high temperature conditions.

Following the four-week treatment period, tissues were collected and freeze dried for 24 hours, then extracted three times using 0.75 mL of 100% methanol. Each extraction was carried out at 70° C. for 15 minutes. Extracts were subjected to chloroform purification by adding 500 μL extract to 355 μL of water and 835 μL chloroform. Following centrifugation, the upper phase was collected and freeze dried, then dissolved in deionized water. Glutamic acid and arginine content were determined using L-Glutamic acid and Arginine kits (Megazyme Intl., Bray, Ireland) according to the manufacturer's instructions.

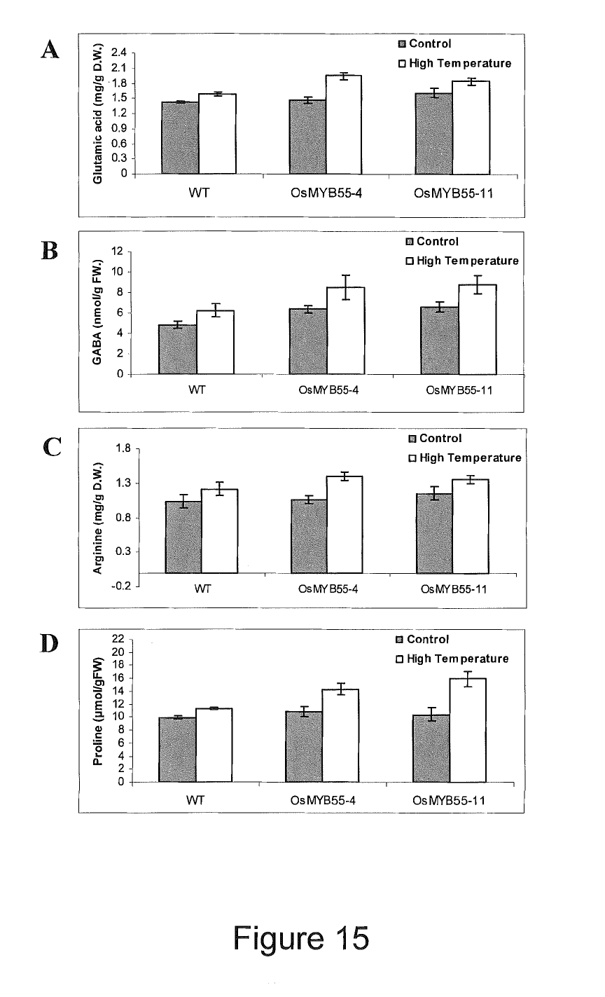

Glutamic acid is one of the first amino acids to be synthesized from nitrogen compounds and can be converted into other amino acids. Consistent with the finding that transgenic rice plants overexpressing OsMYB55 have higher OsGS1;2 transcript levels than wild-type rice plants when grown under normal growth condition, transgenic rice plants overexpressing OsMYB55 had a higher root glutamic acid content than wild-type rice plants when grown under normal growth conditions. The glutamic acid content of the leaves of transgenic rice plants overexpressing OsMYB55 was similar to that of wild-type rice plants under normal growth conditions (FIG. 15A). Growing the plants under high temperature conditions increased the glutamic acid content of the leaves and leaf sheathes of both wild-type rice plants and transgenic rice plants overexpressing OsMYB55, but the increase was significantly higher in the transgenic rice plants overexpressing OsMYB55 as compared to their wild-type counterparts (FIG. 15A).

Arginine is required for polyamine biosynthesis in plants, which has been reported to be involved in several plant development and stress conditions, including high temperature (Alcazar et al., BIOTECH. LETT. 28:1867 (2006); Cheng et al., J. INTEGRATIVE PLANT BIOL. 51:489 (2009)). Our results showed that leaves of transgenic rice plants overexpressing OsMY855 had the same level of arginine as those of wild-type rice plants when grown under normal temperature conditions for four weeks (FIG. 15C). Growing the plants under high temperature conditions increased the arginine content of the leaves of both wild-type rice plants and transgenic rice plants overexpressing OsMYB55, but the increase was significantly higher in the transgenic rice plants overexpressing OsMYB55 (FIG. 15C).

### Example 16

### OsMYB55 Overexpression Enhances Leaf GABA Content

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under long daylight conditions with either normal temperatures conditions or high temperature conditions.

Plant tissues were collected and GABA content was determined as described by Zhang and Bown, PLANT J. 44:361 (2005). Briefly, 0.1 g of frozen tissue was extracted with 400 μl methanol at 25° C. for 10 minutes. The samples were vacuum dried, and dissolved in 1 ml of 70 mM lanthanum chloride. The samples were then shaken for 15 minutes, centrifuged at 13,000×g for 5 minutes, and 0.8 ml of the supernatant removed to a second 1.5 ml tube. To this was added 160 μl of 1 M KOH, followed by shaking for 5 min, and centrifugation as before. The resulting supernatant was used in the spectrophotometric GABA assay described below.

The 1 ml assay contained 550 μl of a sample, 150 μl 4 mM NADP+, 200 μl 0.5 M K+pyrophosphate buffer (prepared by adding 0.15 M phosphoric acid drop-wise to reach the pH 8.6), 50 μl of 2 units GABASE per ml and 50 μl of 20 mM α-ketoglutarate. The initial A was read at 340 nm before adding α-ketoglutarate, and the final A was read after 60 min. The difference in A values was used to construct a calibration graph. The commercial GABASE enzyme preparation was dissolved in 0.1 M K-Pi buffer (pH 7.2) containing 12.5% glycerol and 5 mM 2-mercaptoethanol. The resulting solution was frozen until use.

In this study, under normal temperature conditions, we found an increase in the leaf GABA content of the transgenic rice plants overexpressing OsMYB55 compared to their wild-type counterparts (FIG. 15B). Growing the plants under high temperature conditions increased the GABA content of the leaves of both wild-type rice plants and transgenic rice plants overexpressing OsMYB55, but the increase was significantly more obvious in the transgenic rice plants overexpressing OsMYB55 (FIG. 15B).

### Example 17

### OsMYB55 Overexpression Enhances Leaf Proline Content

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions until shortly after germination (10 days after planting), then grown for four weeks under long daylight conditions with either normal temperatures conditions or high temperature conditions.

Plant tissues were collected and proline content was determined according to the protocol previously reported by Ábrahám et al., METHODS MOL. BIOL. 639:317 (2010). Briefly, 100 mg frozen tissues were extracted by 500 μL 3% sulfosalicylic acid and the supernatant was used for proline quantification. A reaction mixture of 200 μL of glacial acetic acid and 200 μL acidic ninhydrin was added to 200 μL of extract. The reaction was incubated at 96° C. for 60 minutes and terminated in ice. Proline was extracted from the samples in 1 mL toluene and the absorbance in the upper phase was measured at 520 nm after centrifugation. Proline concentration was determined using a standard curve and calculated on fresh weigh basis.

In this study, no significant difference was found in proline content between wild-type rice plants and transgenic rice plants overexpressing OsMYB55 under normal temperature conditions (FIG. 15D). Growing the plants under high temperature conditions increased the proline content of the leaves of both wild-type rice plants and transgenic rice plants overexpressing OsMYB55, but the increase was significantly higher in the transgenic rice plants overexpressing OsMYB55 (FIG. 15D).

### Example 18

### Microarray Hybridization and Data Analysis

Seeds from wild-type rice plants and transgenic rice plants overexpressing OsMYB55 were planted in 500 ml pots. Plants were grown in growth cabinets (Conviron, Manitoba, Canada) under full-nutrient conditions using 1 g of a slow-release fertilizer containing nitrogen, phosphorus and potassium (13-13-13) and supplemented with micronutrients. Plants were grown under normal growth conditions for four weeks, and then exposed to 45° C. for one hour. Leaves of the wild-type and transgenic rice plants were harvested, and total RNA was isolated.

Double-stranded cDNAs were synthesized from 5 μg of total RNA from each sample. Labeled complementary RNA, synthesized from the cDNA was hybridized to a rice whole genome array (Cat. No. 900601, Affymetrix, Inc., Santa Clara, Calif.). The hybridization signal of the arrays was obtained by the GeneChip Scanner 3000 (Affymetrix, Inc., Santa Clara, Calif.) and quantified by Microarray Suite 5.0 (Affymetrix, Inc., Santa Clara, Calif.). The probe set 25 measurement was summarized as a value of weighted average of all probes in a set, subtracting the bottom 5% of average intensity of the entire array using a custom algorithm. The overall intensity of all probe sets of each array was further scaled to a target intensity of 100 to enable direct comparison. Data was analyzed using GeneSpring software (Agilent Technologies, Santa Clara, Calif.). Genes with two-fold change were identified first, and then ANOVA was used to identify significant genes (Welch t-test p-value cutoff at 0.05).

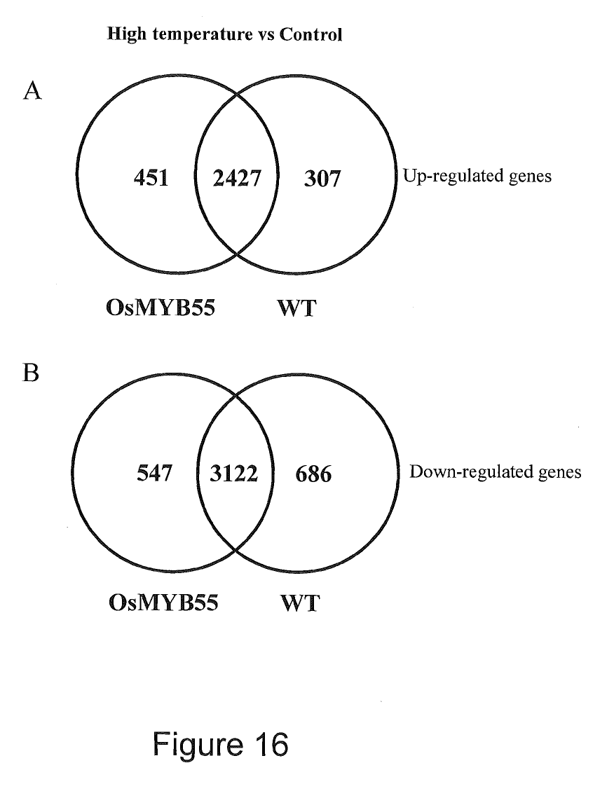

As shown in FIGS. 16A-16B, numerous genes were found to be up-regulated and/or down-regulated in response to high growth temperatures, both in wild-type rice plants and in transgenic rice plants overexpressing OsMYB55.

### Example 19

### Statistical Analyses

All statistical analyses were performed using SigmaStat (SPSS Inc., Chicago, Ill.) with an error rate set at α=0.05. The significance difference between treatments was tested using Tukey's Honestly Significant Difference Test.

### Example 20

### Discussion

We identified a MYB transcription factor that enhances rice plant tolerance to high temperature during the vegetative growth stage. The overexpression of OsMYB55 improved plant growth and productivity under high temperature conditions. The transgenic plants maintain higher plant height and more dry-biomass as compared with the wild-type plants grown under high temperature. Exposure of the wild-type plants for four weeks in the first six weeks of the life cycle to high temperature decreased grain yield at harvest. However, this reduction was significantly less in the transgenic plants. Together, these results indicate that the transgenic lines grow and perform better under high temperature than wild-type. Although, there was a positive effect of OsMYB55 overexpression in plant heat tolerance, a RNAi knockdown of its expression did not show any significant difference from wild-type. This is likely due to the high level of redundancy in this gene family, although it could be due to the fact that these lines still had some expression, albeit lower, of the OsMYB55 gene.

To explore the function of the OsMYB55 in enhancing plant growth under high temperature, different biochemical and molecular analyses have been carried out. Histological analysis of the leaf, leaf sheath and stem tissues of the wild-type and transgenic plants growing at high temperature did not reveal any significant difference (data not shown), suggesting that the higher plant height and leaf sheath length are results of enhancing general plant performance rather than affecting cell expansion or division. In addition, biochemical analysis of starch, sugars and hydrogen peroxide as an indicator for antioxidant activity did not show significant changes in the transgenic plants compared to the wild-type plants (data not shown). Rivero et al., PLANT BIOL. (STUTTG) 6:702 (2004), suggested the importance of nitrogenous compounds and proline in plant heat tolerance. We investigated nitrate and quaternary ammonium compounds in the wild-type and transgenic plants growing at two different temperatures and no significant difference was found (data not shown).

In the present study, plants that overexpressed OsMYB55 had an increase in total amino acid content. Transcript analysis of several Heat Shock Transcription Factors and Heat Shock Proteins known to be involved in plant responses to high temperature revealed no significant difference between the wild-type and the transgenic plants (data not shown). Global transcriptome analysis did result in the identification of three targets for the rice OsMYB55, namely OsGS1;2, GAT1 and GAD3. OsMYB55 binds in vitro to the promoters of these genes and transactivates them in tobacco leaf cells.

OsMYB55 overexpression leads to improved heat tolerance and enhances the level of both total amino acids and glutamic acid, proline, arginine, and GABA in particular. It should be noted that while proline content was increased in the overexpression lines in response to high temperature, there were no significant differences in the expression of the genes involved in proline biosynthesis. These results suggest that the increase in proline content is indirect and could be due to other pathways including protein breakdown as suggested by Becker and Fock, PHOTOSYNTHESIS RES. 8:267 (1986).

In conclusion, overexpression of OsMYB55 leads to increased heat tolerance of rice plants during the vegetative stage. This leads to increased biomass and, if the plants are subsequently grown under normal conditions, increased seed yield. This trait will become of increasing importance as crop yields in many important rice growing regions are decreased due to higher temperatures given global warming. Therefore, it is of great importance to explore different crop genetic solutions to ameliorate this problem. Modulating the expression of OsMYB55 either by itself or in combination with other genes is one potential solution.

The foregoing is illustrative of the present invention, and is not to be construed as limiting thereof. The invention is defined by the following claims, with equivalents of the claims to be included therein.

All publications, patent applications, patents, database accession numbers and other references cited herein are incorporated by reference in their entireties for the teachings relevant to the sentence and/or paragraph in which the reference is presented.

1-30. (canceled)

31. A method of increasing amino acid content in a transgenic plant or plant part, comprising:
introducing a nucleic acid encoding a MYB55 polypeptide into a plant or plant part to produce a transgenic plant or plant part that expresses the nucleic acid encoding the MYB55 polypeptide and exhibits increased amino acid content as compared to a control plant or plant part.

32. The method of claim 1, wherein introducing the nucleic acid encoding the MYB55 polypeptide into the plant or plant part to produce the transgenic plant or plant part that expresses the nucleic acid encoding the MYB55 polypeptide and exhibits increased amino acid content as compared with the control plant or plant part comprises:
(a) introducing the nucleic acid into a plant cell to produce a transgenic plant cell; and
(b) regenerating a transgenic plant or plant part from the transgenic plant cell, wherein the transgenic plant or plant part comprises the nucleic acid within its genome and exhibits increased amino acid content as compared to the control plant or plant part.

33. The method of claim 1, wherein introducing the nucleic acid encoding the MYB55 polypeptide into the plant or plant part to produce the transgenic plant or plant part that expresses the nucleic acid encoding the MYB55 polypeptide and exhibits increased amino acid content as compared with the control plant or plant part comprises:
(a) introducing the nucleic acid into a plant cell to produce a transgenic plant cell;
(b) regenerating a plurality of transgenic plants or plants part from the transgenic plant cell; and
(c) selecting, from the plurality of transgenic plants or plant parts of (b), a transgenic plant or plant part that exhibits increased amino acid content as compared to the control plant or plant part.

34. The method of claim 1, further comprising:
obtaining a progeny plant or plant part from the transgenic plant or plant part that expresses the nucleic acid encoding the MYB55 polypeptide and exhibits increased amino acid content as compared to a control plant or plant part,
wherein the progeny plant or plant part comprises the nucleic acid within its genome and exhibits increased amino acid content.

35. The method of claim 1, wherein the amount(s) and/or concentration(s) of glutamic acid, arginine, gamma-amino butyric acid and/or proline is/are increased in the transgenic plant or plant part.

36. The method of claim 1, wherein the nucleic acid is introduced as part of an expression cassette that comprises the nucleic acid in operable association with a promoter that functions in plant cells, and wherein the expression cassette optionally comprises a selectable marker.

37. The method of claim 1, wherein the increased amino acid content is exhibited in a leaf, leaf sheath and/or root of the transgenic plant or plant part.

38. The method of claim 1, wherein the nucleic acid encoding the MYB55 polypeptide comprises:
(a) the nucleotide sequence of any one of SEQ ID NOs: 3-4 or 14-20;
(b) a nucleotide sequence that is at least 95% identical to the nucleotide sequence of any one of SEQ ID NOs: 3-4 or 14-20;
(c) a nucleotide sequence that encodes the amino acid sequence of any one of SEQ ID NOs: 5-13;
(d) a nucleotide sequence that encodes an amino acid sequence that is at least 95% identical to the amino acid sequence of any one of SEQ ID NOs: 5-13; or
(e) a nucleotide sequence that hybridizes to a complete complement of any one of (a) to (d) above under stringent hybridization conditions.

39. The method of claim 1, wherein the nucleic acid encoding the MYB55 polypeptide comprises a nucleotide sequence that encodes the amino acid sequence of SEQ ID NO: 5.

40. The method of claim 1, wherein the nucleic acid encoding the MYB55 polypeptide comprises the nucleotide sequence of SEQ ID NO: 4 and/or nucleotides 4062 to 5126 of SEQ ID NO: 3.

41. The method of claim 1, wherein the introducing is via bacterial-mediated transformation, particle bombardment transformation, calcium-phosphate-mediated transformation, cyclodextrin-mediated transformation, electroporation, liposome-mediated transformation, nanoparticle-mediated transformation, polymer-mediated transformation, virus-mediated nucleic acid delivery, whisker-mediated nucleic acid delivery; microinjection, sonication, infiltration, polyethyleneglycol-mediated transformation, or a combination thereof.

42. The method of claim 1, wherein the plant or plant part is a monocotyledonous plant or plant part.

43. The method of claim 1, wherein the plant or plant part is a dicotyledonous plant or plant part.

44. The method of claim 1, wherein the plant or plant part is rice, maize, wheat, barley, sorghum, oat, rye, sugar cane, soybean or Arabidopsis.

45. A transgenic plant or plant part produced by the method of claim 1, wherein the transgenic plant or plant part expresses the nucleic acid encoding the MYB55 polypeptide and exhibits increased amino acid content as compared to a control plant or plant part.
